(19) United States
(12) Patent Application Publication
(10) Pub. No.: US 2022/0340532 A1
(43) Pub. Date: Oct. 27, 2022

(54) PROCESSES FOR THE PREPARATION OF ROXADUSTAT AND INTERMEDIATES THEREOF

(71) Applicant: TEVA PHARMACEUTICALS INTERNATIONAL GMBH, Jona (CH)

(72) Inventors: Parven Kumar Luthra, Thane (IN); Suhail Ahmad, Noida (IN); Neval Kishor, Gonda (IN); Miteshgir Kanchangir Goswami, Vadodara (IN); Alok Singh, Varanasi (IN); Chandrasekhar Sinha, Navi Mumbai (IN); Syed Aziz Imam Quadri, Hyderabad (IN); Prashant Shankar Joshi, Ratnagiri (IN); Sadanand Hardeo Maurya, Thane (IN)

(21) Appl. No.: 17/628,681

(22) PCT Filed: Aug. 6, 2020
(86) PCT No.: PCT/US20/45128

§ 371 Date: Jan. 20, 2022

(30) Foreign Application Priority Data
Aug. 7, 2019 (IN) 201911032049
Nov. 18, 2019 (IN) 201911046891

(51) Int. Cl. C07D 217/26

(52) U.S. Cl. CPC C07D 217/26

(57) ABSTRACT

The present invention provides new procedure and intermediates for the preparation of Roxadustat (1) comprising: (A) reducing a compound of formula 3′, 3″ or a mixture thereof: (3′), (3″) wherein Pg is H or a OH protecting group, Ri is alkyl, aryl, or arylalkyl; R₂, R₃, R₄, and Rs each independently represents alkyl, arylalkyl or alkenyl, or R₂ and R₃ and/or R₄ and Rs, taken together with the nitrogen atom to which they are bonded, each independently form a ring selected from: (I), wherein R₆ is H or C_{I-6} alkyl; R₇ is Ci to C6 alkyl and X— is an anion selected from the group consisting of halide, O—SO₄—R₇ wherein R₇ is Ci to C₆ alkyl, or O—SO₂-Rs wherein Rs is phenyl, tolyl, methyl or trifluoromethyl; to form a compound of formula (2′) wherein Pg is H or an OH protecting group, Ri is alkyl, aryl, or arylalkyl; and removing the Ri group and where present removing the OH protecting group; or (B) reducing a compound of formula 4′, a compound of formula 4″ or a mixture thereof: (4′), (4″) wherein Pg is H or an OH protecting group; Ri is H, alkyl, aryl, or arylalkyl; R₂, R₃, R₄, and R5 each independently represents alkyl, aryl, arylalkyl or alkenyl; or R₂ is Ci-₄ alkyl and R₃ is Ci-₄ alkoxy; or R₂ and R₃ and/or R₄ and R₅, taken together with the nitrogen atom to which they are bonded, each independently form a group selected from: (I) wherein R₆ is H or C_{I-6} alkyl; and where Ri is not H, removing the Ri group, and, where present removing the OH protecting group.

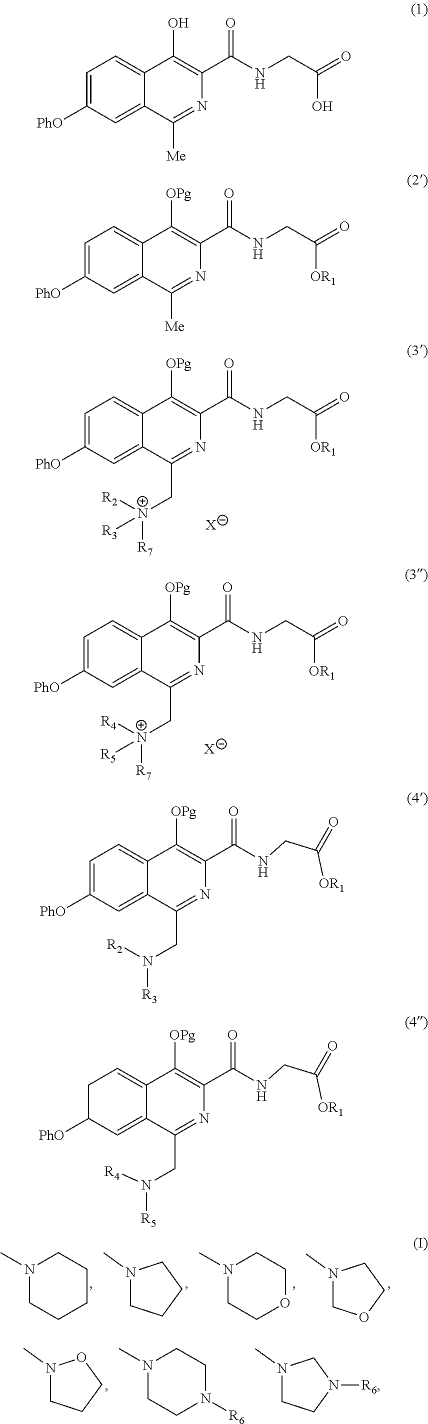

### FIELD OF THE INVENTION

The present invention provides new procedure and intermediates for the preparation of Roxadustat.

### BACKGROUND OF THE INVENTION

Roxadustat which has the chemical name N-[(4-hydroxy-1-methyl-7-phenoxy-3-isoquinolinyl) carbonyl]-glycine is a potent inhibitor of hypoxia inducible factor (HIF) prolyl hydroxylase, as described in U.S. Pat. No. 7,323,475. Roxadustat has the following chemical structure:

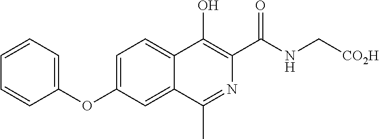 [SMILES: Cc1nc(C(=O)NCC(=O)O)c(O)c2ccc(Oc3ccccc3)cc12]

HIF prolyl hydroxylase inhibitors are useful for increasing the stability and/or activity of HIF, and useful for, inter alia, treating and preventing disorders associated with HIF, including anemia, ischemia, and hypoxia.

Roxadustat preparation and intermediates thereof is disclosed in U.S. Pat. No. 7,323,475 (example D81), as demonstrated in the following scheme A:

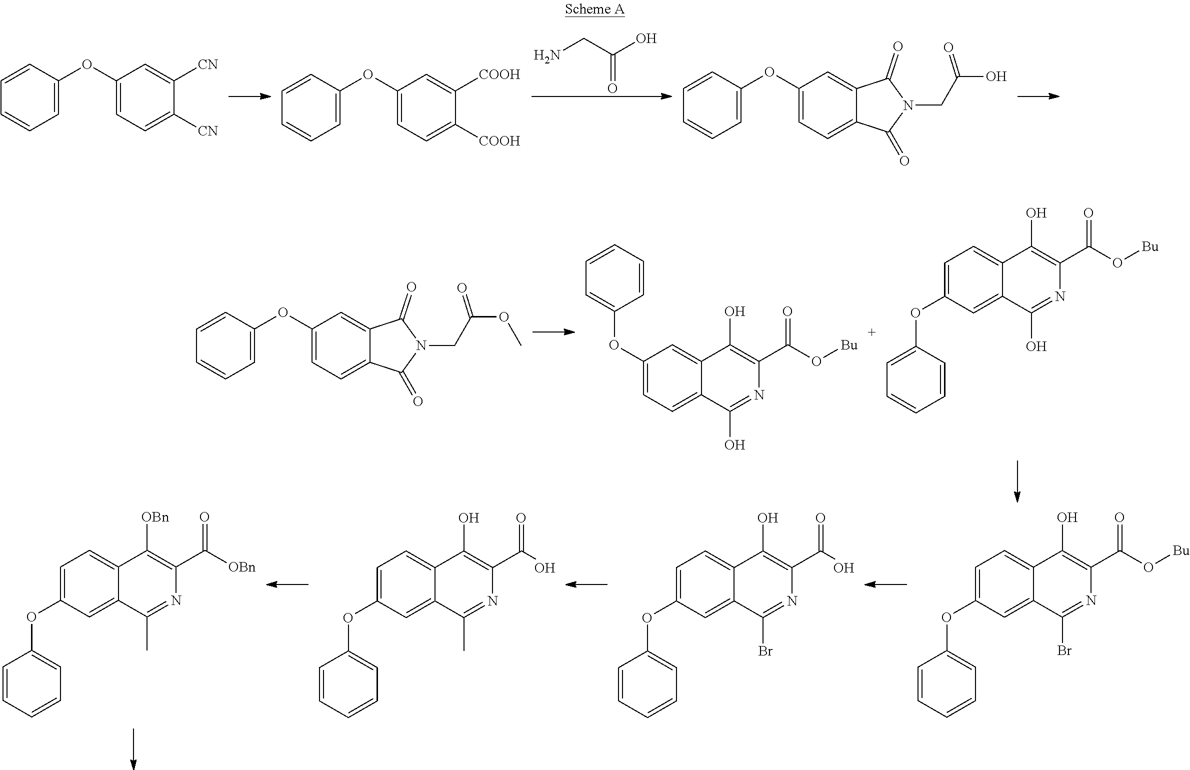

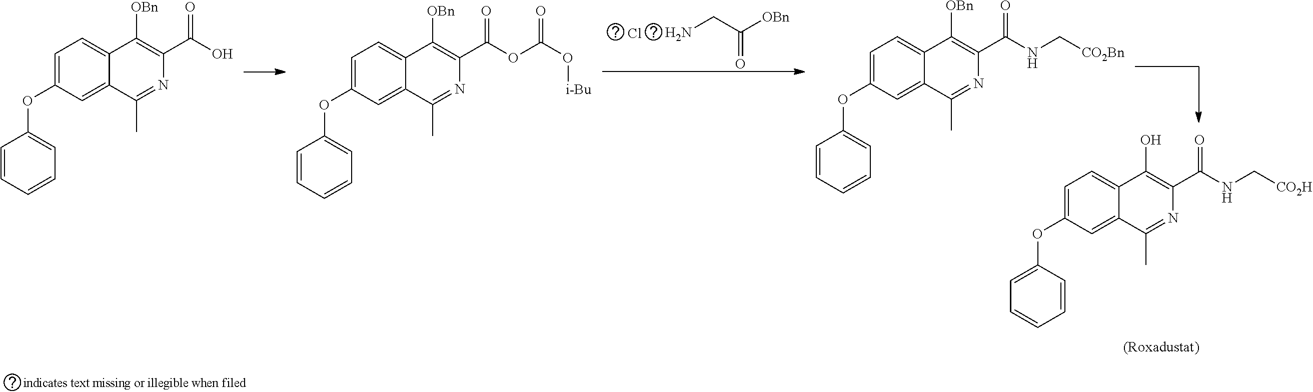

The process described above in U.S. Pat. No. 7,323,475 has significant disadvantages, since this process shows low isomeric selectivity and low yield.

Another U.S. Pat. Nos. 9,340,511 and 9,708,269 describe different process for the preparation of Roxadustat, as demonstrated by scheme B.

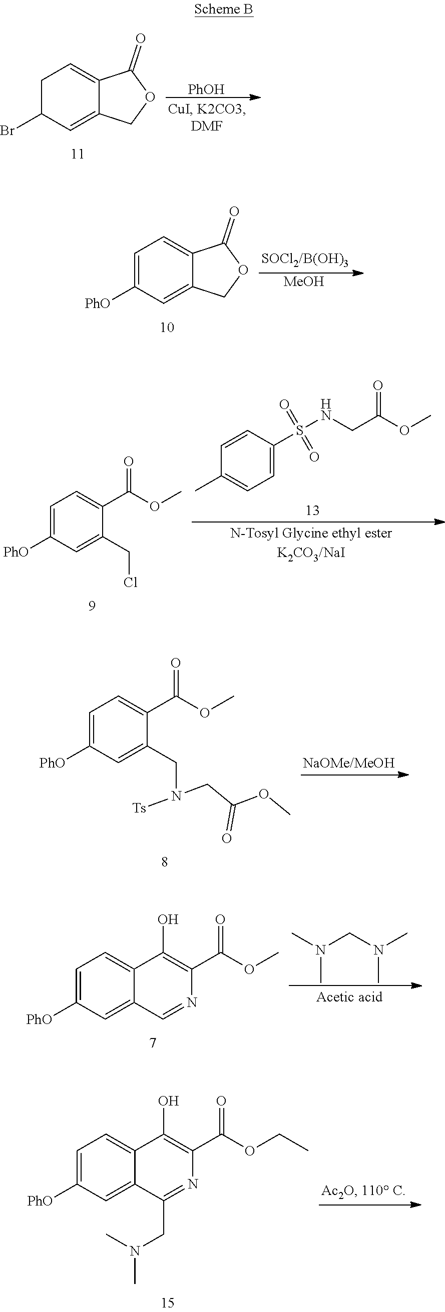

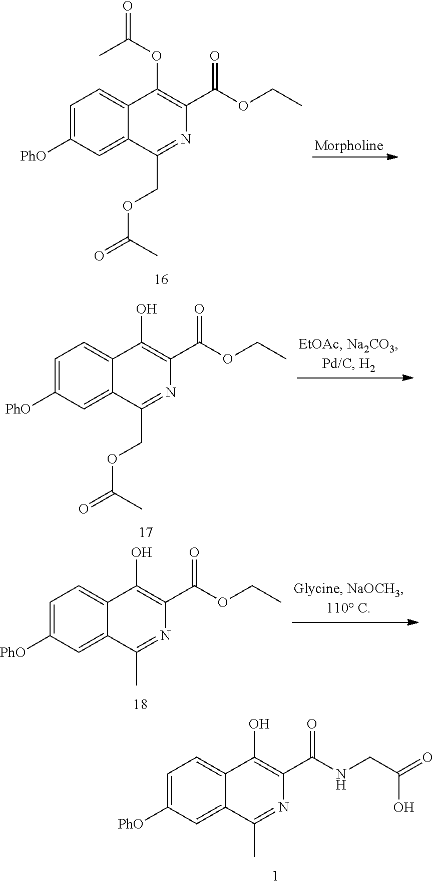

The process described above in U.S. Pat. Nos. 9,340,511 and 9,708,269 is not preferable since it includes many stages of synthesis that affects the complexity and the high cost of this procedure in production scale. Furthermore, the conversion of compound 17 to 18 (as defined above) involves special equipment such as pressure reactor and costly palladium catalyst under hydrogen gas which is not preferable procedure that can be used in high quantity production scale.

Another U.S. Pat. No. 9,206,134 describes also preparation of Roxadustat, as demonstrated by scheme C below.

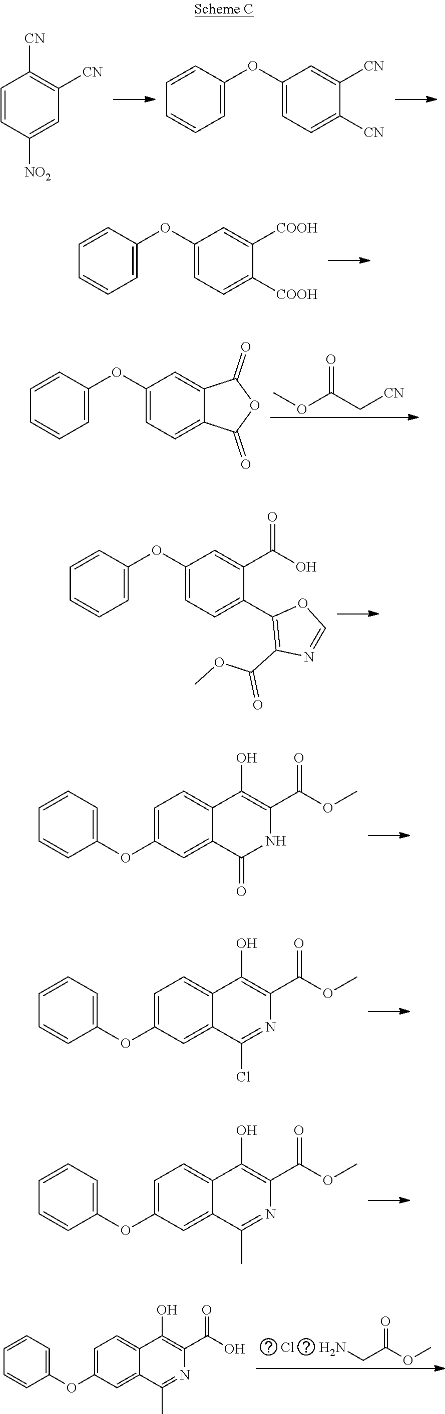

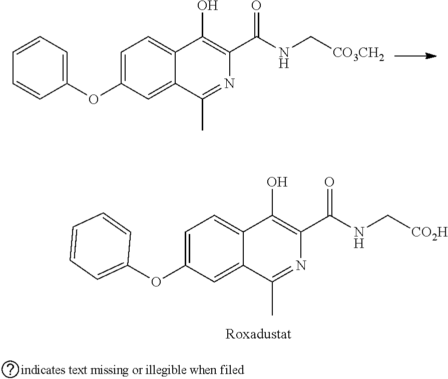

The main disadvantage of the above described process (U.S. Pat. No. 9,206,134) is its low regioselectivity which resulted with low yield of Roxadustat.

Recently new International Publication No. WO 2019/106621 described another process for Roxadustat, as demonstrated by scheme D below.

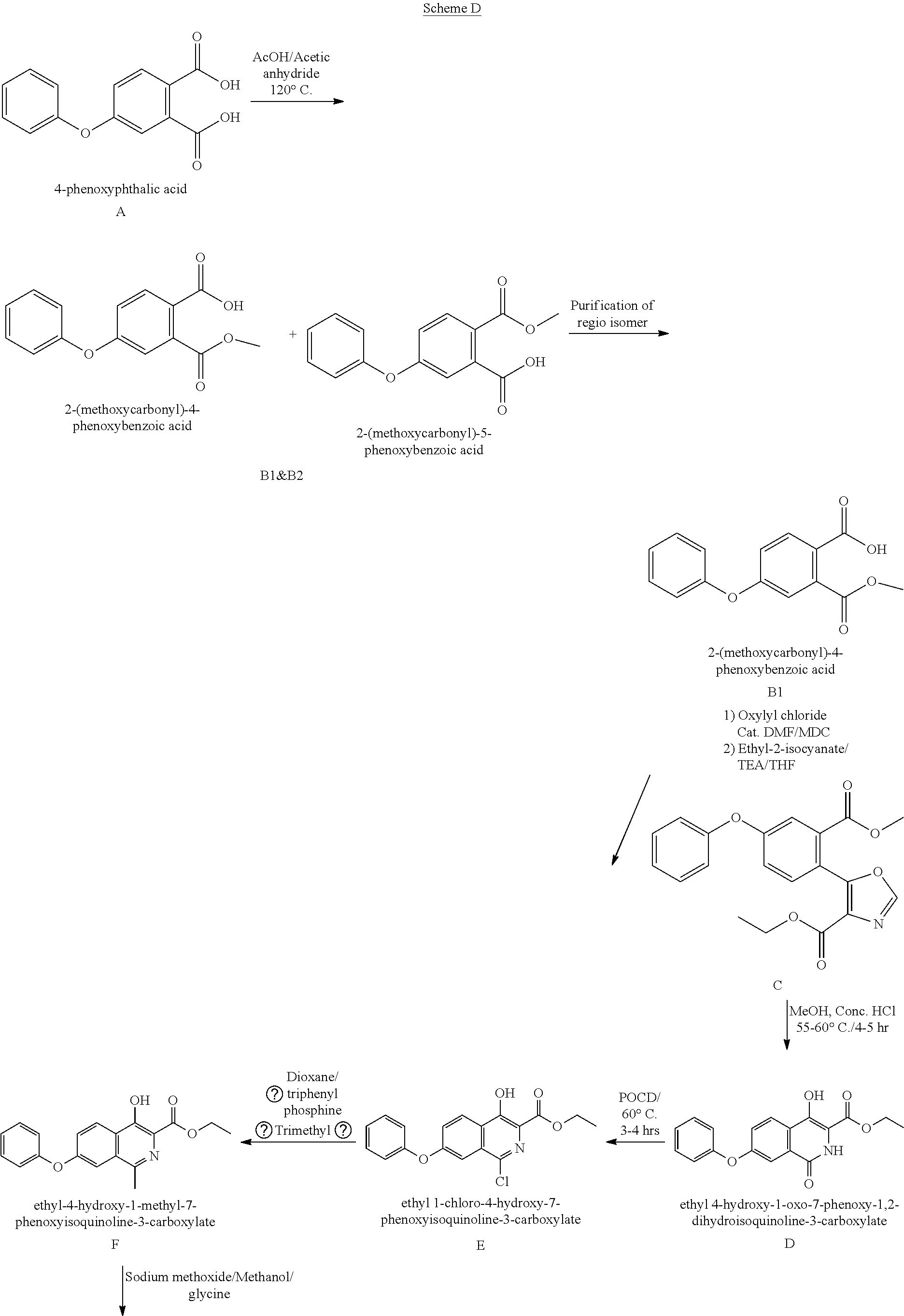

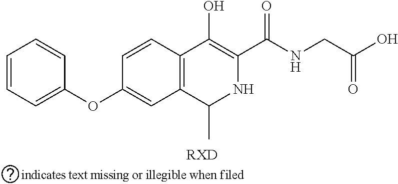

The methylation of the 4-phenoxyphthalic acid generates two regio isomers, which are purified to get B1 therefore the theoretical yield of this process is only 50%. Furthermore, the conversion of B1 to C involves the highly flammable ethyl isocyanate which evolves the acute toxic gases which is not safe and must be controlled. In addition, the methylation reaction was performed by Suzuki coupling reaction from intermediate E to F with tetrakis triphenyl phosphine palladium and Trimethyl boroxin in dioxane which is genotoxic solvent and should be avoided.

Thus, it is the object of the present invention to devise other improved methods of synthesizing Roxadustat that lack the disadvantages of the procedures described in the prior art. Hence, there is a need for procedure which involves only few synthetic steps, and provides Roxadustat in high yield and quality, that can be utilized in industrial scale.

### SUMMARY OF THE DISCLOSURE

The present disclosure provides new procedure and intermediates for the preparation of Roxadustat.

In another aspect, the present disclosure provides novel intermediates, their preparation and their use in the preparation of Roxadustat.

According yet to another aspect the present invention relates to novel process by using the intermediate of formula (7′), preferably intermediate of formula (7) for the preparation of intermediate formula (6′), preferably intermediate of formula (6)

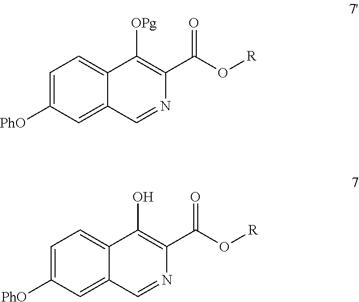

wherein R is H, substituted or unsubstituted alkyl, substituted or unsubstituted arylalkyl, or acid protecting group.
Wherein Pg is H or —OH protecting group.

According to yet another aspect the present invention relates to novel process by using the intermediate of formula (6′), preferably intermediate of formula (6) for the preparation of intermediate formula (5′), preferably intermediate of formula (5)

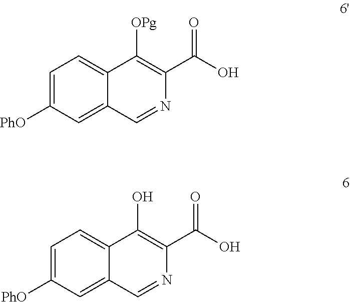

wherein Pg is H or —OH protecting group.

According to another aspect the present invention relates to novel process by using the intermediate of formula (7′), preferably formula (7) for the preparation of formula (5′), preferably formula (5).

According to another aspect the present invention relates to novel process by combining the intermediate of formula (8) and formula (9) for the preparation of intermediate of formula (5′), preferably intermediate of formula (5).

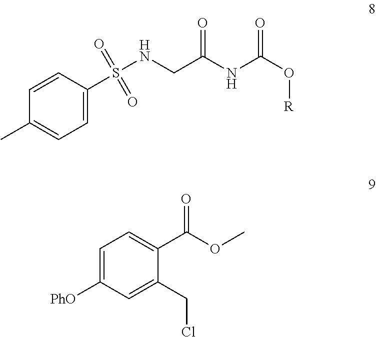

wherein R is H, substituted or unsubstituted alkyl, substituted or unsubstituted arylalkyl, or acid protecting group.

According to yet another aspect the present invention relates to novel process by using the intermediate of formula (5′), preferably intermediate of formula (5) for the preparation of intermediate formula (4′), preferably intermediate of formula (4)

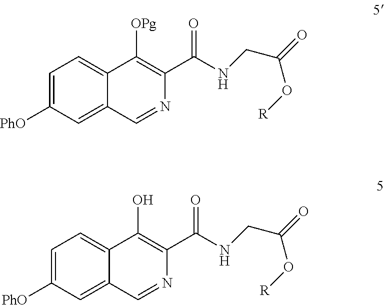

wherein R is H, substituted or unsubstituted alkyl, substituted or unsubstituted arylalkyl, or acid protecting group.
wherein Pg is H or —OH protecting group.

According to yet another aspect the present invention relates to novel process by using the intermediate of formula (4′), preferably intermediate of formula (4) for the preparation of intermediate formula (3′), preferably for the preparation of intermediate of formula (3)

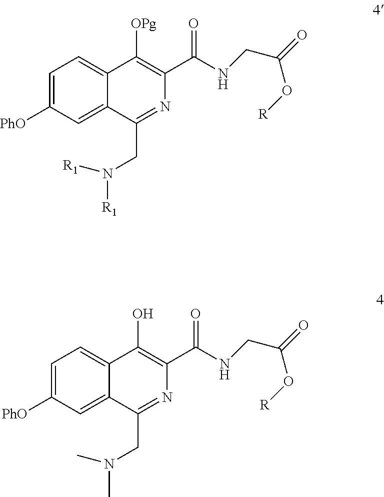

wherein R is H, substituted or unsubstituted alkyl, substituted or unsubstituted arylalkyl, or acid protecting group.
wherein R¹ is H, substituted or unsubstituted alkyl, substituted or unsubstituted arylalkyl, or amine protecting group or one R¹ is alkyl and other R¹ is oxygen attached to alkyl group or hydrogen or two R¹ together with the nitrogen atom to which they are attached to form 4-8 membered heterocyclic like 1-Methylpiperazine, piperidine, pyrrolidine, morpholine or hetetroaromatic.
wherein Pg is H or —OH protecting group.

According to yet another aspect the present invention relates to novel process by using the intermediate of formula (3′), preferably formula (3) for the preparation of intermediate formula (2′) and formula (2) respectively.

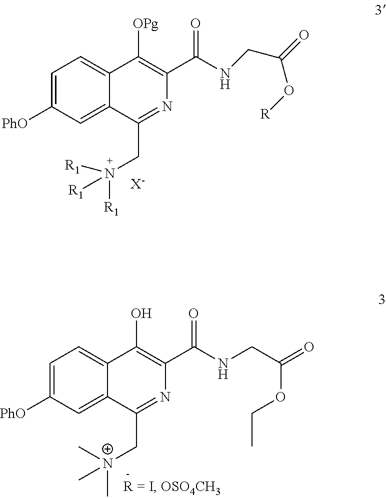

wherein R is H, substituted or unsubstituted alkyl, substituted or unsubstituted arylalkyl, or acid protecting group.
wherein R¹ is H, substituted or unsubstituted alkyl, substituted or unsubstituted arylalkyl, or amine protecting group. wherein Pg is H or —OH protecting group.
wherein X⁻ is anion of corresponding amine preferably Iodo or methanesulfate.

According to yet another aspect the present invention relates to novel process by using the intermediate of formula (2′), preferably using intermediate of formula (2) for the preparation Roxadustat.

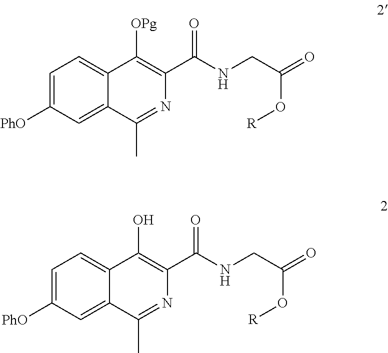

wherein R is H, substituted or unsubstituted alkyl, substituted or unsubstituted arylalkyl, or acid protecting group
wherein Pg is H or —OH protecting group.

According to yet another aspect the present invention relates to novel process by using the intermediate of formula (5′), preferably formula (5), more preferably intermediate of formula (5-methyl) and formula (5-acid) for the preparation of intermediate formula (4a′), preferably intermediate of formula (4a) and formula (4b).

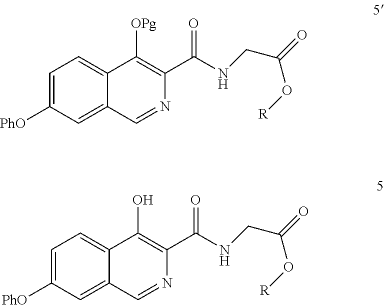

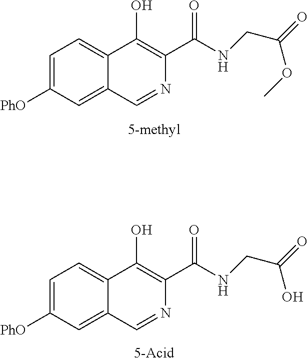

wherein R is H, substituted or unsubstituted alkyl, substituted or unsubstituted arylalkyl, or acid protecting group, preferably H or methyl.
wherein Pg is H or —OH protecting group.

According to yet another aspect the present invention relates to novel process by using the intermediate of formula (4a′), preferably intermediate of formula (4a) and formula (4b) for the preparation of intermediate formula (2′), preferably for the preparation of intermediate of formula (2) or for the preparation of Roxadustat.

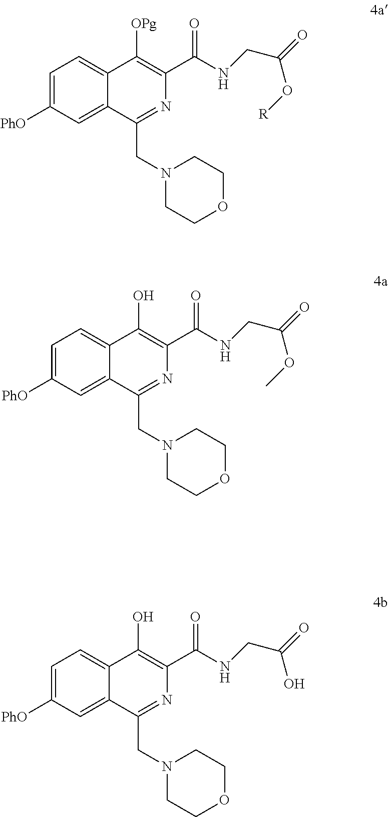

wherein Pg is H or —OH protecting group.
wherein R is H, substituted or unsubstituted alkyl, substituted or unsubstituted arylalkyl, or acid protecting group.

According to yet another aspect the present invention relates to novel process by using the intermediate of formula (5′) preferably formula (5) and more preferably intermediate of formula (5-methyl) and formula (5-acid) for the preparation of intermediate formula (4c′) preferably intermediate of formula (4c) and formula (4d).

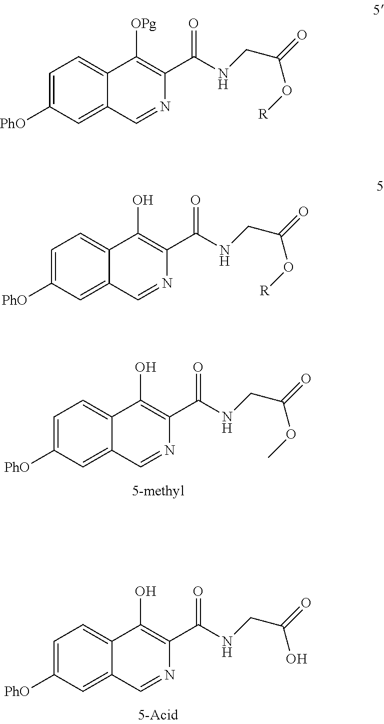

wherein R is H, substituted or unsubstituted alkyl, substituted or unsubstituted arylalkyl, or acid protecting group. Preferably methyl and H.
wherein Pg is H or —OH protecting group.

According to yet another aspect the present invention relates to novel process by using the intermediate of formula (4c′) preferably intermediate of formula (4c) and formula (4d) for the preparation of intermediate formula (2′), preferably for the preparation of intermediate of formula (2) and for the preparation of Roxadustat.

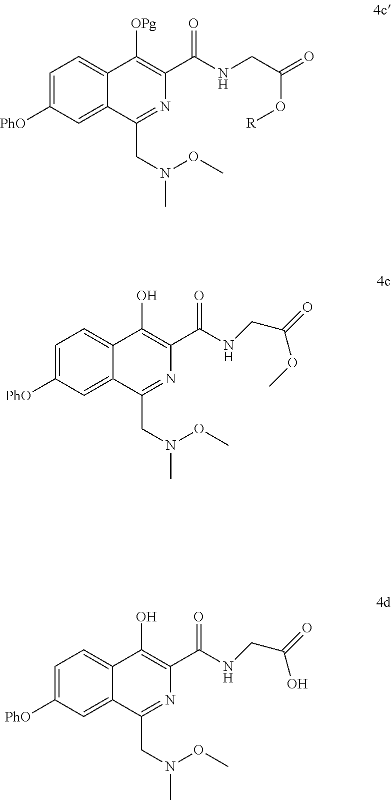

wherein Pg is H or —OH protecting group.
wherein R is H, substituted or unsubstituted alkyl, substituted or unsubstituted arylalkyl, or acid protecting group.

According to yet another aspect the present invention relates to novel process by using the intermediate of formula (5′), preferably formula (5), more preferably intermediate of formula (5-methyl) and formula (5-acid) for the preparation of intermediate formula (4e′), preferably intermediate of formula (4e) and formula (4f).

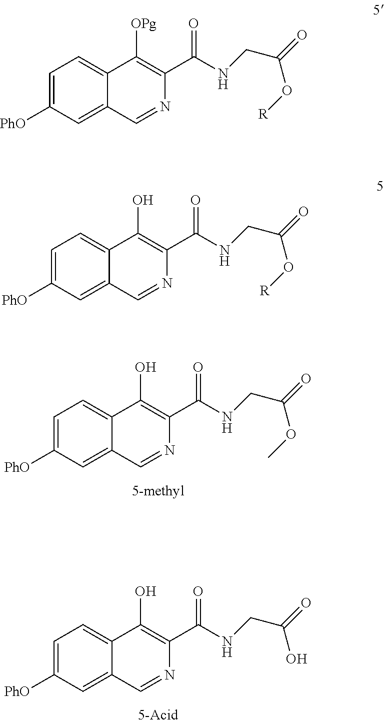

wherein R is H, substituted or unsubstituted alkyl, substituted or unsubstituted arylalkyl, or acid protecting group. Preferably methyl and H.
wherein Pg is H or —OH protecting group.

According to yet another aspect the present invention relates to novel process by using the intermediate of formula (4e′) preferably intermediate of formula (4e) and formula (4f) for the preparation of intermediate formula (2′), preferably for the preparation of intermediate of formula (2) and for the preparation of Roxadustat.

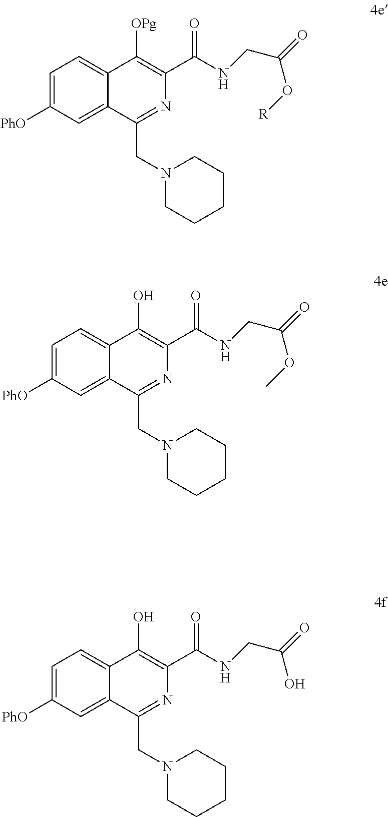

wherein R is H, substituted or unsubstituted alkyl, substituted or unsubstituted arylalkyl, or acid protecting group. Preferably methyl and H.
wherein Pg is H or —OH protecting group.

In yet further aspect the present disclosure provides novel intermediate of formula (5′), preferably formula (5) that can be used in the preparation of formula (4′), preferably formula (4)

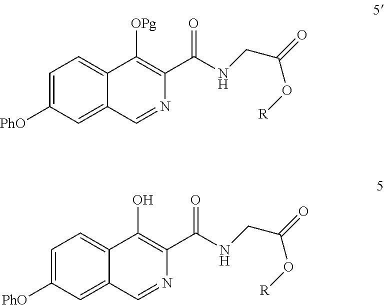

wherein R is H, substituted or unsubstituted alkyl, substituted or unsubstituted arylalkyl, or acid protecting group
wherein Pg is H or —OH protecting group.

In yet further aspect the present disclosure provides novel intermediate of formula (4′), preferably formula (4) that can be used in the preparation of formula (3′), preferably formula (3).

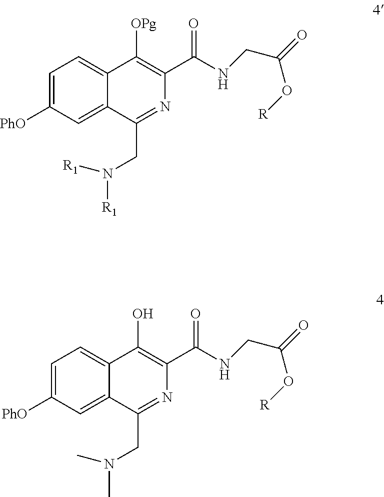

wherein R is H, substituted or unsubstituted alkyl, substituted or unsubstituted arylalkyl, or acid protecting group.
wherein R¹ is H, substituted or unsubstituted alkyl, substituted or unsubstituted arylalkyl, or amine protecting group, or one R¹ is alkyl and other R¹ is oxygen attached to alkyl group or hydrogen or two R¹ together with the nitrogen atom to which they are attached to form 4-8 membered heterocyclic like 1-Methylpiperazine, piperidine, pyrrolidine, morpholine or hetetroaromatic.
wherein Pg is H or —OH protecting group.

In yet further aspect the present disclosure provides novel intermediate of formula (3′) and formula (3) that can be used in the preparation of Roxadustat.

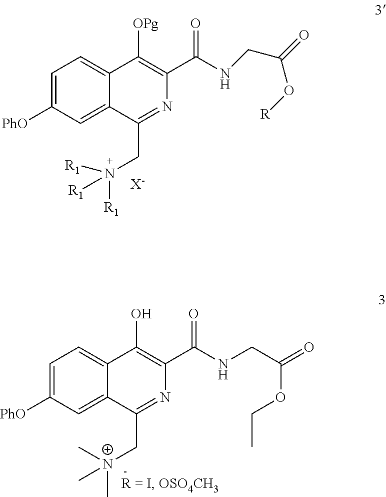

wherein R is selected from the list consisting of substituted or unsubstituted alkyl, substituted or unsubstituted arylalkyl, or acid protecting group. Preferably R is selected from the group consisting of methyl, ethyl, t-butyl and benzyl, most preferably R is methyl.
wherein X⁻ is anion of corresponding amine preferably Iodo or methanesulfate.
wherein R¹ is H, substituted or unsubstituted alkyl, substituted or unsubstituted arylalkyl, or amine protecting group or one R¹ is alkyl and other R¹ is oxygen attached to alkyl group or hydrogen or two R¹ together with the nitrogen atom to which they are attached to form 4-8 membered heterocyclic like 1-Methylpiperazine, piperidine, pyrrolidine, morpholine or hetetroaromatic.
wherein Pg is H or —OH protecting group.

In yet further aspect the present disclosure provides novel intermediate of formula (4a′), preferably formula (4a) and formula (4b) that can be used in the preparation of formula (2′) preferably formula (2) and in the preparation of Roxadustat.

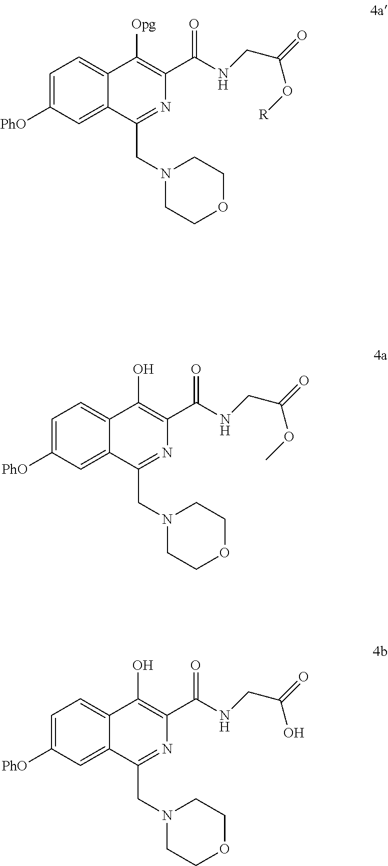

wherein R is H, substituted or unsubstituted alkyl, substituted or unsubstituted arylalkyl, or acid protecting group. wherein Pg is H or —OH protecting group.

In yet further aspect the present disclosure provides novel intermediate of formula (4c′), preferably formula (4c) and formula (4d) that can be used in the preparation of formula (2′), preferably formula (2) and in the preparation of Roxadustat.

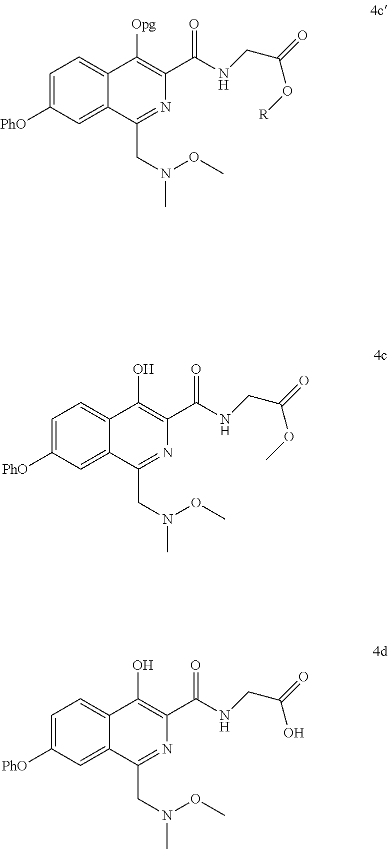

wherein R is H, substituted or unsubstituted alkyl, substituted or unsubstituted arylalkyl, or acid protecting group, wherein Pg is H or —OH protecting group.

In yet further aspect the present disclosure provides novel intermediate of formula (4e′), preferably formula (4e) and formula (4f) that can be used in the preparation of formula (2′) preferably formula (2) and in the preparation of Roxadustat.

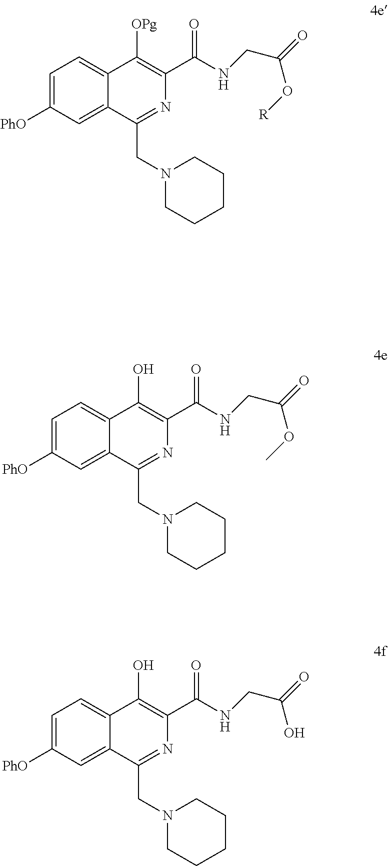

wherein R is H, substituted or unsubstituted alkyl, substituted or unsubstituted arylalkyl, or acid protecting group, wherein Pg is H or —OH protecting group.

According yet to another aspect, the present disclosure provides the novel compounds (3′), (4′) and (5′) preferably (3), (4) and (5).

According yet to another aspect, the present disclosure provides the novel compounds (4a′), (4c′) and (4e′), preferably (4a), (4b), (4c), (4d), (4e) and (4f).

In another aspect the present disclosure provides the use of any one of the compounds of formula (3′), (4′) and (5′) preferably (3), (4) and (5) in the preparation of Roxadustat.

In another aspect the present disclosure provides the use of any one of the compounds of formula (4a′), (4c′) and (4e′), preferably (4a), (4b), (4c). (4d), (4e) and (4f) in the preparation of Roxadustat.

In another aspect the present disclosure provides any one of compounds of formula (3′), (4′) and (5′) preferably (3), (4) and (5) for use in the preparation of Roxadustat.

In another aspect the present disclosure provides any one of compounds of formula (4a′), (4c′) and (4e′), preferably (4a), (4b), (4c), (4d), (4e) and (4f) for use in the preparation of Roxadustat

The present disclosure provides for novel processes for preparation of any one of compounds (3′), (4′) and (5′) preferably (3), (4) and (5) and for the preparation of Roxadustat.

The present disclosure provides for novel processes for preparation of any one of compounds (4a′), (4c′) and (4e′), preferably (4a), (4b), (4c). (4d), (4e) and (4f) for the preparation of Roxadustat.

The present disclosure provides for novel processes for preparation of compound (2′) from compound (3′) preferably compound (2) from compound (3).

In yet further aspect the present disclosure provides novel intermediate of formula (5′), preferably formula (5) that can be used in the preparation of formula (4′), preferably formula (4)

In yet further aspect the present disclosure provides novel intermediate of formula (5′), preferably formula (5) that can be used in the preparation of formula (4a′), (4c′) and (4e′), preferably (4a), (4b), (4c), (4d), (4e) and (4f).

In another embodiment the compound of formula (5′) preferably formula (5) is prepared by combining formula (8) and formula (10).

In yet another embodiment the compound of formula (5′) preferably formula (5) used above can be prepared by combining the intermediate of formula (8) and formula (9) to afford formula (10) followed by isoquinoline ring cyclisation

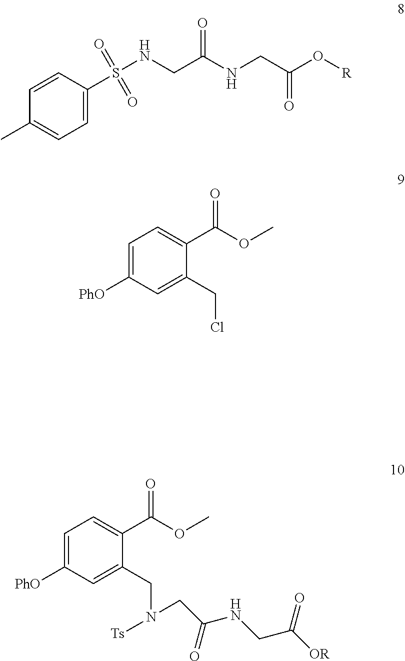

wherein R is selected from H, substituted or unsubstituted alkyl, substituted or unsubstituted arylalkyl, or acid protecting group, preferably H or methyl.

In yet another embodiment the disclosure relates to novel process for preparation of Roxadustat comprising

a) converting formula (7′), preferably formula (7)

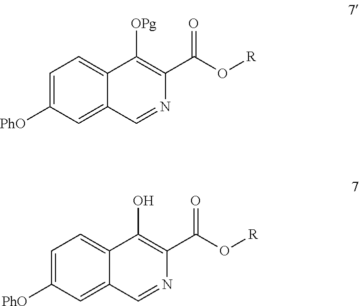

wherein R is H, substituted or unsubstituted alkyl, substituted or unsubstituted arylalkyl, or acid protecting group.
wherein Pg is H or —OH protecting group.
to afford the compound of formula (6′), preferably formula (6)

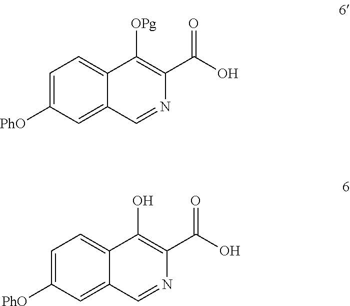

wherein Pg is H or —OH protecting group; and
b) converting the compound of formula (6′) preferably formula (6) to afford the compound of formula (5′) preferably formula (5)

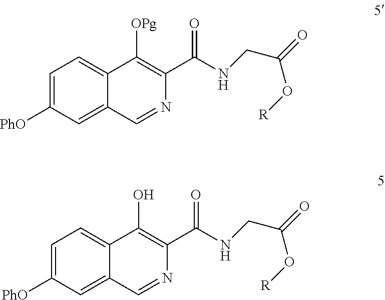

wherein R is H, substituted or unsubstituted alkyl, substituted or unsubstituted arylalkyl, or acid protecting group.
wherein Pg is H or —OH protecting group. alternatively,
c) converting Formula (7′) preferably formula (7) to obtain formula (5′) preferably, formula (5)
alternatively,
d) combining the intermediate of formula (8) and formula (9) to afford formula (10) followed by isoquinoline ring cyclisation to obtain formula (5′) preferably formula (5)

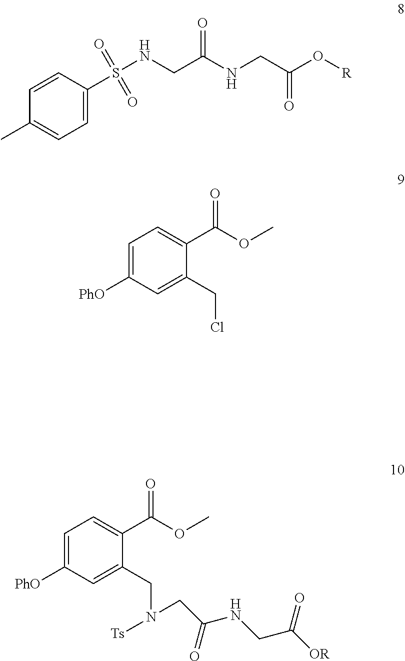

wherein R is selected from H, substituted or unsubstituted alkyl, substituted or unsubstituted arylalkyl, or acid protecting group, preferably H or methyl; and
e) converting the compound of formula (5′), preferably formula (5) to afford compound of formula (4′), preferably formula (4)

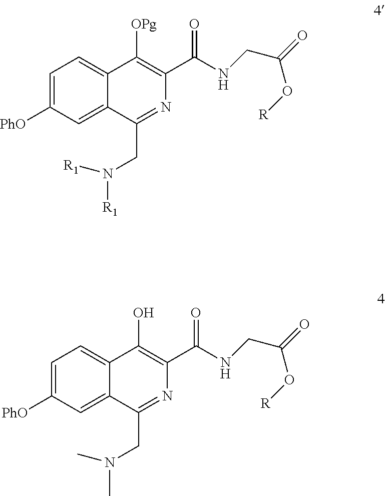

wherein R¹ is H, substituted or unsubstituted alkyl, substituted or unsubstituted arylalkyl, or amine protecting group, or one R¹ is alkyl and other R¹ is oxygen attached to alkyl group or hydrogen or two R¹ together with the nitrogen atom to which they are attached to form 4-8 membered heterocyclic like 1-Methylpiperazine, piperidine, pyrrolidine, morpholine or hetetroaromatic.
wherein R is H, substituted or unsubstituted alkyl, substituted or unsubstituted arylalkyl, or acid protecting group.
wherein Pg is H or —OH protecting group.
f) converting compound of formula (4′), preferably formula (4) to afford compound of formula (3′), preferably formula (3)

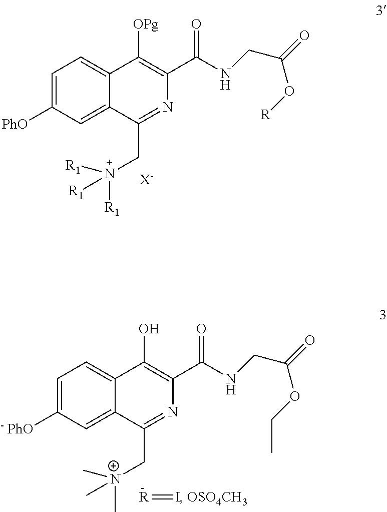

wherein R¹ is H, substituted or unsubstituted alkyl, substituted or unsubstituted arylalkyl, or amine protecting group.
wherein Pg is H or —OH protecting group.
wherein X⁻ is anion of corresponding amine.
g) converting compound of formula (3′), preferably formula (3) to afford compound of formula (2′), preferably formula (2)

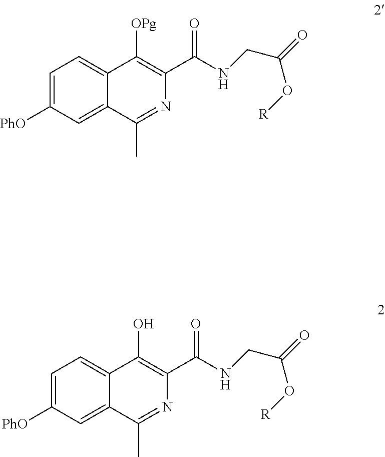

wherein R is H, substituted or unsubstituted alkyl, substituted or unsubstituted arylalkyl, or acid protecting group.
wherein Pg is H or —OH protecting group.
and optionally
h) converting compound of formula (2′) preferably formula (2) to Roxadustat.

In yet another embodiment the disclosure relates to novel process for preparation of Roxadustat comprising:

a) converting compound of formula (7′) preferably formula (7) to compound of formula (5′) preferably formula (5) using formula (6′) preferably formula (6) according to steps a and b described above, or alternatively, converting compound of formula (7′) preferably formula (7) to compound of formula (5′) preferably formula (5)
alternatively,
b) combining the intermediate of formula (8) and formula (9) followed by isoquinoline ring cyclisation to obtain formula (5′) preferably formula (5); and
c) converting compound of formula (5′) preferably formula (5) to afford compound of formula (4a′) preferably formula (4a) and formula (4b)

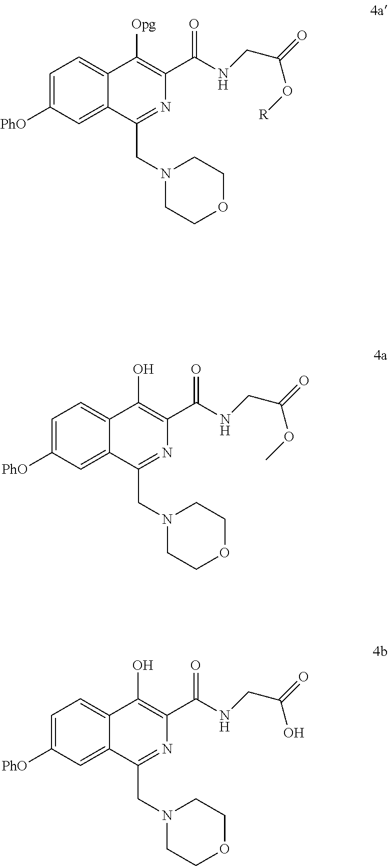

wherein Pg is H or —OH protecting group.
wherein R is H, substituted or unsubstituted alkyl, substituted or unsubstituted arylalkyl, or acid protecting group.
and
d) converting compound of formula (4a′) preferably formula (4a) and formula (4b) to afford compound of formula (2′) preferably formula (2)

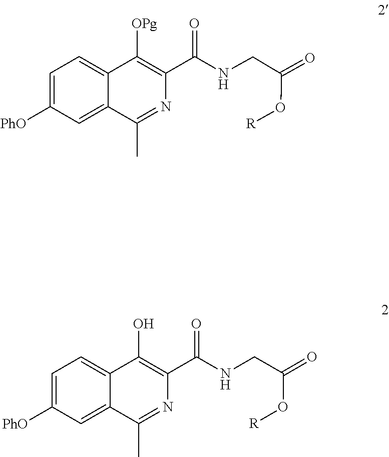

wherein R is H, substituted or unsubstituted alkyl, substituted or unsubstituted arylalkyl, or acid protecting group, preferably methyl or H.
and;
e) converting compound of formula (2′) preferably formula (2) to Roxadustat

In yet another embodiment the disclosure relates to novel process for preparation of Roxadustat comprising:

a. converting compound of formula (7′) preferably formula (7) to compound of formula (5′) preferably formula (5) using formula (6) preferably formula (6′), or converting compound of formula (7′) preferably formula (7) to compound of formula (5′) preferably formula (5) alternatively,
b. combining the intermediate of formula (8) and formula (9) to obtain formula (5′) preferably formula (5).
and;
c. converting compound of formula (5′) preferably formula (5) to afford compound of formula (4c′) preferably formula (4c) and formula (4d)

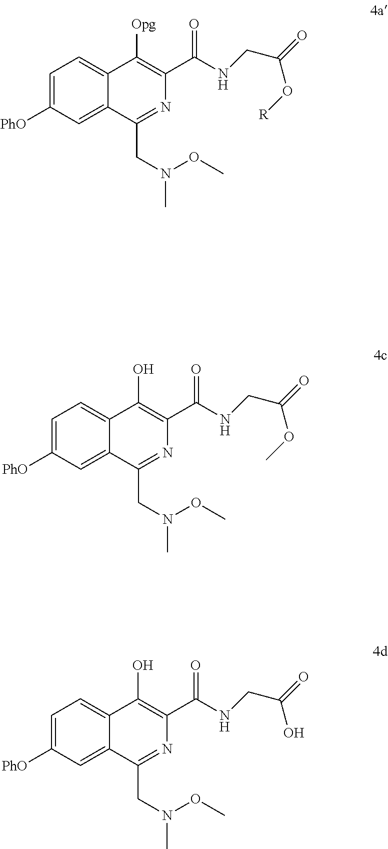

wherein Pg is H or —OH protecting group.
wherein R is H, substituted or unsubstituted alkyl, substituted or unsubstituted arylalkyl, or acid protecting group.
and;
d. converting compound of formula (4c′) preferably formula (4c) and formula (4d) to afford compound of formula (2′) preferably formula (2)

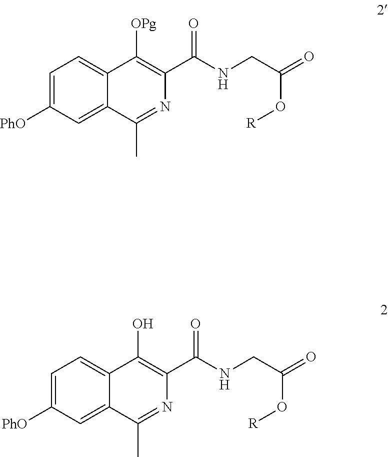

wherein R is H, substituted or unsubstituted alkyl, substituted or unsubstituted arylalkyl, or acid protecting group, preferably methyl or H.
wherein Pg is H or —OH protecting group.
and;
e. converting compound of formula (2′) preferably compound of formula (2) to Roxadustat.

In yet another embodiment the disclosure relates to novel process for preparation of Roxadustat comprising:

a. converting compound of formula (7′) preferably formula (7) to compound of formula (5′) preferably formula (5) using formula (6′) preferably formula (6), or converting compound of formula (7′) preferably formula (7) directly to compound of formula (5′) preferably formula (5) alternatively,
b. combining the intermediate of formula (8) and formula (9) to afford formula (5′) preferably formula (5).
and;
c. converting compound of formula (5′) preferably formula (5) to afford compound of formula (4e′) preferably formula (4e) and formula (4f)

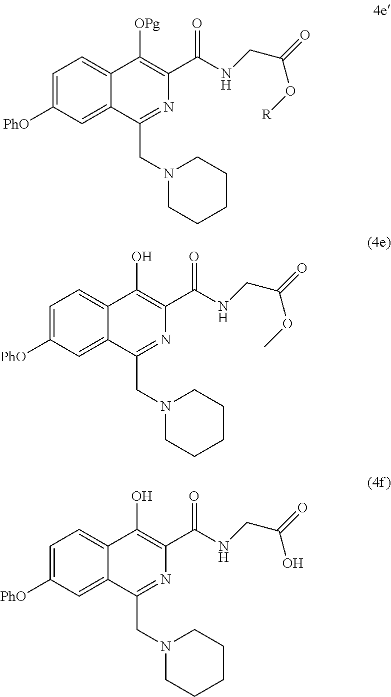

wherein R is H, substituted or unsubstituted alkyl, substituted or unsubstituted arylalkyl, or acid protecting group. Preferably methyl and H.
wherein Pg is H or —OH protecting group.
and
d. converting compound of formula (4e′) preferably formula (4e) and formula (4f) to afford compound of formula (2′) preferably formula (2)
and;
e. converting compound of formula (2′) preferably formula (2) to Roxadustat

In yet another embodiment the disclosure relates to novel process for preparation of Roxadustat comprising—

a) converting formula (7′), preferably formula (7) under basic conditions, preferably with sodium hydroxide

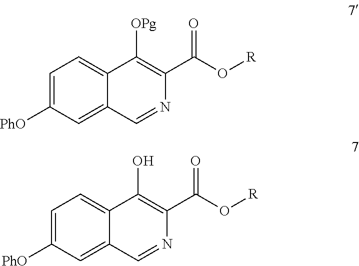

wherein R is H, substituted or unsubstituted alkyl, substituted or unsubstituted arylalkyl, or acid protecting group.
wherein Pg is H or —OH protecting group.
to afford formula (6′), preferably formula (6)

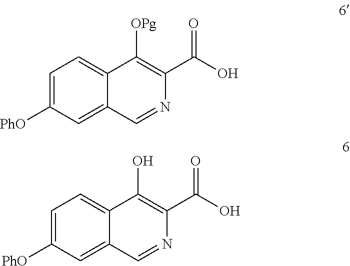

wherein Pg is H or —OH protecting group.
and
b) reacting the compound of formula (6′) preferably formula (6), with Glycine ethyl ester or salt thereof to afford the compound of formula (5′), preferably formula (5)

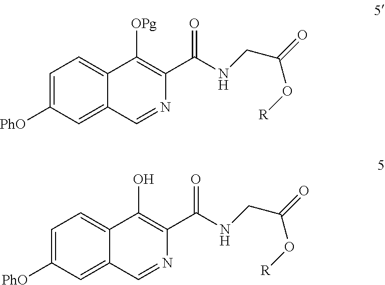

wherein R is H, substituted or unsubstituted alkyl, substituted or unsubstituted arylalkyl, or acid protecting group.
wherein Pg is H or —OH protecting group.

### Alternatively,

c) reacting the compound of formula (7′) preferably formula (7) with Glycine ethyl ester or salt thereof to afford formula (5′) preferably formula (5)
and
d) reacting the compound of formula (5′) preferably formula (5) with N,N,N′,N′-Tetra alkylmethanediamine reagent such as N,N,N′,N′-Tetramethylmethanediamine to afford compound of formula (4′), preferably formula (4)

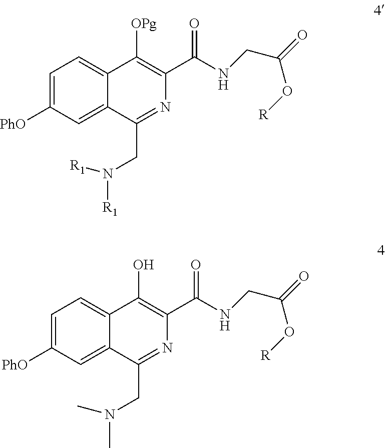

wherein R is H, substituted or unsubstituted alkyl, substituted or unsubstituted arylalkyl, or acid protecting group.
wherein R¹ is H, substituted or unsubstituted alkyl, substituted or unsubstituted arylalkyl, or amine protecting group or one R¹ is alkyl and other R¹ is oxygen attached to alkyl group or hydrogen or two R¹ together with the nitrogen atom to which they are attached to form 4-8 membered heterocyclic like 1-Methylpiperazine, piperidine, pyrrolidine, morpholine or hetetroaromatic.
wherein Pg is H or —OH protecting group.
e) alkylation of compound (4′), preferably compound (4) to afford compound of formula (3′), preferably formula (3)

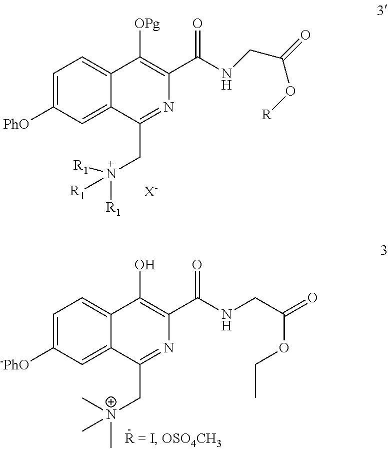

wherein alkylation reagent can be selected from methyl iodide, Di methyl sulfate (DMS), dimethyl carbonate (DMC), methyl bromide. Preferably methyl iodide, Di methyl sulfate (DMS).
wherein R is H, substituted or unsubstituted alkyl, substituted or unsubstituted arylalkyl, or acid protecting group.
wherein R¹ is H, substituted or unsubstituted alkyl, substituted or unsubstituted arylalkyl, or amine protecting group.

Wherein two R¹ together with the nitrogen atom to which they are attached to form 4-8 membered heterocyclic like 1-Methylpiperazine, piperidine, pyrrolidine, morpholine or hetetroaromatic.

wherein Pg is H or —OH protecting group.
and
f) reduction compound of formula (3′) preferably formula (3) to afford formula (2′), preferably formula (2)

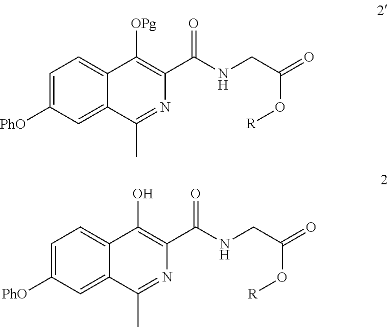

wherein R is H, substituted or unsubstituted alkyl, substituted or unsubstituted arylalkyl, or acid protecting group.
wherein Pg is H or —OH protecting group.
and
g) converting formula (2′), preferably formula (2) to Roxadustat.

In another embodiment intermediate formula (5′) preferably formula (5) used above can alternatively be prepared by reacting the intermediate of formula (8) and formula (9) with halide of alkali and alkaline earth metals selected from sodium iodide, potassium iodide, potassium bromide and preferably sodium iodide under basic conditions to afford formula (10), followed by isoquinoline ring cyclisation with alkoxide of alkali and alkaline earth metals, preferably sodium methoxide, potassium tertiary butoxide, sodium tertiary butoxide, sodium amylate, potassium amylate, preferably sodium methoxide

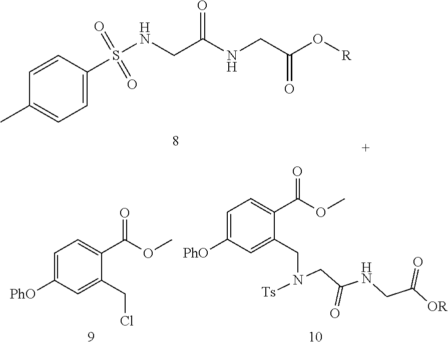

wherein R is H, substituted or unsubstituted alkyl, substituted or unsubstituted arylalkyl, or acid protecting group, preferably H or methyl.

In yet another embodiment the disclosure relates to novel process for preparation of Roxadustat comprising—

a. coupling formula (7′), preferably formula (7) with Glycine to afford compound of formula (5′) preferably formula (5) more preferably compound of formula (5-acid)

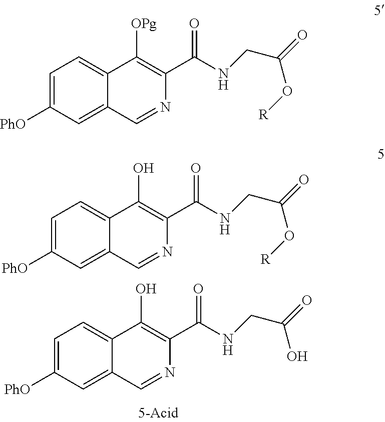

wherein R is H, substituted or unsubstituted alkyl, substituted or unsubstituted arylalkyl, or acid protecting group, preferably H or methyl

wherein Pg is H or —OH protecting group.

alternatively,
b. reacting the intermediate of formula (8) and formula (9) with halide of alkali and alkaline earth metals, preferably sodium iodide, potassium iodide, potassium bromide and sodium iodide, more preferably sodium iodide, under basic environment to afford formula (10), followed by isoquinoline ring cyclisation with alkoxide of alkali and alkaline earth metals, preferably sodium methoxide, potassium tertiary butoxide, sodium tertiary butoxide, sodium amylate, potassium amylate, preferably sodium methoxide to obtain formula (5′) preferably formula (5)
and;
c. reacting the compound of formula (5′) preferably (5) more preferably formula (5-methyl) and formula (5-acid) with morpholine and Paraformaldehyde under acid conditions, preferably glacial acetic acid to afford compound of formula (4a′) preferably formula (4a) and formula (4b).

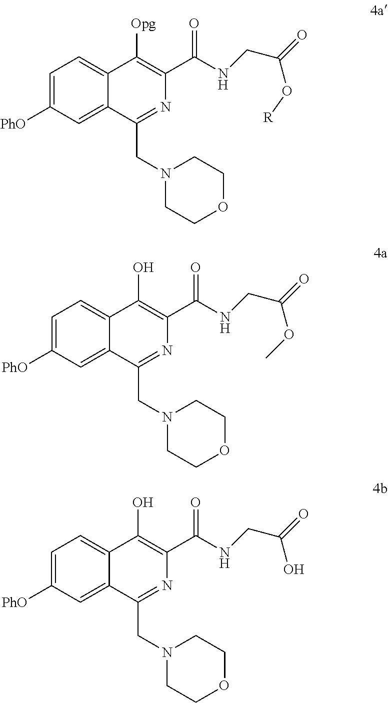

and;
d. reduction compound of formula (4a′) preferably formula (4a) and formula (4b) with reduction reagent selected from Sn, SnCl₂, Fe, Ni, and salt of this metal, Pd/C, Pt, Pd(OH)₂. Sodium/liquid ammonia and zinc, preferably with zinc under acid conditions, preferably with hydrochloric acid to afford formula (2′) preferably formula (2-methyl) and Roxadustat respectively.

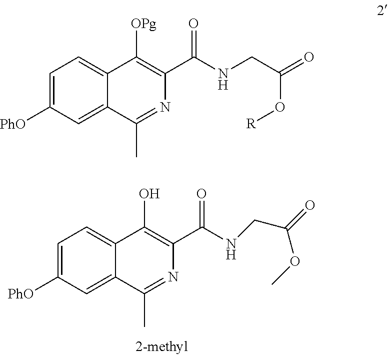

wherein R is H, substituted or unsubstituted alkyl, substituted or unsubstituted arylalkyl, or acid protecting group.
wherein Pg is H or —OH protecting group.
and
e. converting compound of formula (2′) preferably formula (2-methyl) to Roxadustat under basic conditions, preferably using sodium hydroxide.

In yet another embodiment the disclosure relates to novel process for preparation of Roxadustat comprising—

a) coupling formula 7′, preferably formula 7 with Glycine to afford compound of formula (5′) preferably formula (5) more preferably compound of formula (5-acid)

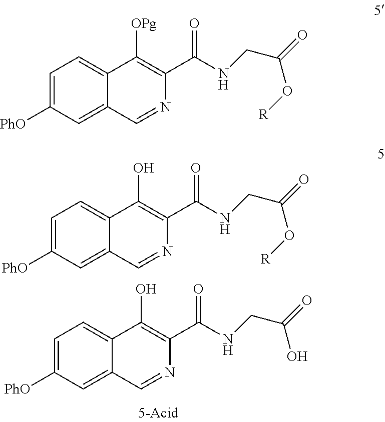

alternatively,
b) reacting the intermediate of formula (8) and formula (9) with halide of alkali and alkaline earth metals, preferably sodium iodide, potassium iodide, potassium bromide and more preferably sodium iodide under basic environment to afford formula (10), followed by isoquinoline ring cyclisation with alkoxide of alkali and alkaline earth metals, preferably sodium methoxide, potassium tertiary butoxide, sodium tertiary butoxide, sodium amylate, potassium amylate, more preferably sodium methoxide to obtain formula (5′) preferably formula (5)
and;
c) reacting the compound of formula (5′) preferably formula (5) more preferably formula (5-methy) and formula (5-acid) with N,O-Dimethylhydroxylamine and paraformaldehyde under acid conditions, preferably glacial acetic acid to afford compound of formula (4c′) and preferably formula (4c) and formula (4d)

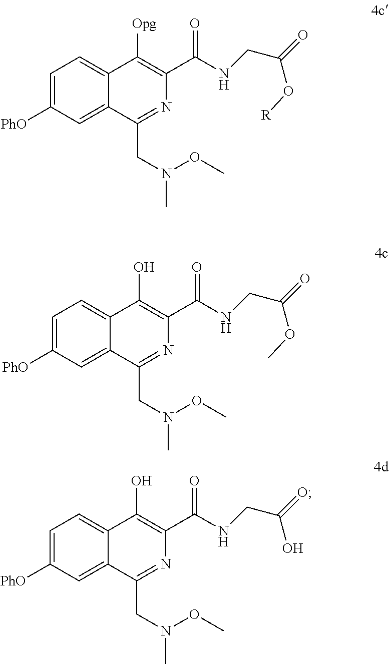

and
d) reduction compound of formula (4c′) preferably formula (4c) and formula (4d) with reduction reagent selected from Sn, SnCl₂, Fe, Ni, and salt of this metal, Pd/C, Pt, Pd(OH)₂. Sodium/liquid ammonia and zinc preferably with zinc under acid conditions, preferably with hydrochloric acid to afford formula afford formula (2′) preferably formula (2-methyl) and Roxadustat respectively.

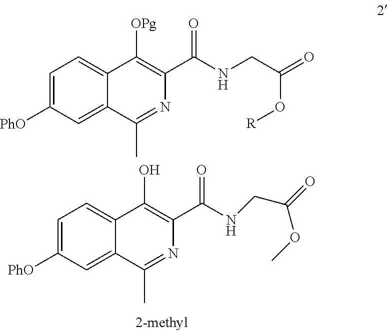

wherein R is H, substituted or unsubstituted alkyl, substituted or unsubstituted arylalkyl, or acid protecting group.
wherein Pg is H or —OH protecting group.
and;
e) converting compound of formula (2′) preferably formula (2-methyl) to Roxadustat under basic conditions, preferably using sodium hydroxide.

In yet another embodiment the disclosure relates to novel process for preparation of Roxadustat comprising—

a) coupling formula (7′), preferably formula (7) with Glycine to afford compound of formula (5′) preferably formula (5) and more preferably formula (5-acid)

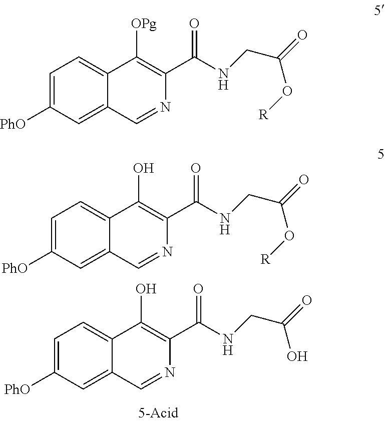

### Alternatively,

b) reacting the intermediate of formula (8) and formula (9) with halide of alkali and alkaline earth metals selected from sodium iodide, preferably sodium iodide, potassium iodide, potassium bromide and more preferably sodium iodide under basic environment to afford formula (10), followed by isoquinoline ring cyclisation with alkoxide of alkali and alkaline earth metals, preferably sodium methoxide, potassium tertiary butoxide, sodium tertiary butoxide, sodium amylate, potassium amylate, and more preferably sodium methoxide to obtain formula (5′) preferably formula (5).
and;
c) reacting the compound of formula (5′) preferably formula (5) more preferably formula (5-methyl) and formula (5-acid) with piperidine and paraformaldehyde under acid conditions, preferably glacial acetic acid to afford compound of formula (4e′) preferably formula (4e) and formula (4f)

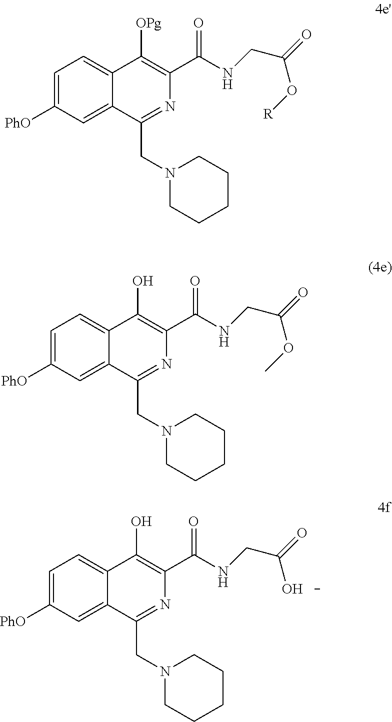

wherein R is H, substituted or unsubstituted alkyl, substituted or unsubstituted arylalkyl, or acid protecting group, preferably H or methyl.
and;
d) reduction compound of formula (4e′) preferably formula (4e) and formula (4f) with reduction reagent selected from Sn, SnCl₂, Fe, Ni, and salt of this metal, Pd/C, Pt, Pd(OH)₂. Sodium/liquid ammonia and zinc preferably with zinc under acid conditions, preferably with hydrochloric acid to afford formula (2′) preferably formula (2-methyl) and Roxadustat.

According to yet another aspect the present invention relates to novel process by using the intermediate of formula (7′), preferably formula (7) for the preparation of intermediate formula (5′) preferably formula (5).

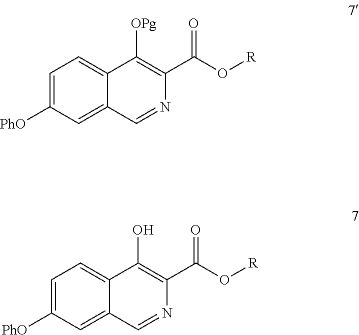

wherein R is selected from the list consisting of substituted or unsubstituted alkyl, substituted or unsubstituted arylalkyl, preferably R is selected from the group consisting of methyl, ethyl, t-butyl and benzyl, most preferably R is methyl.
wherein Pg is H or —OH protecting group.

According to yet another aspect the present invention relates to novel process by using the intermediate of formula (5′) preferably formula (5) for the preparation of intermediate formula (14)

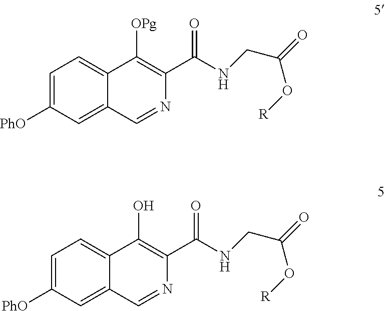

wherein R is H, substituted or unsubstituted alkyl, substituted or unsubstituted arylalkyl, or acid protecting group.
wherein Pg is H or —OH protecting group.

According to yet another aspect the present invention relates to novel process by using the intermediate of formula (14) for the preparation of intermediate formula (4′) preferably formula (4) which can be later used for the preparation of Roxadustat.

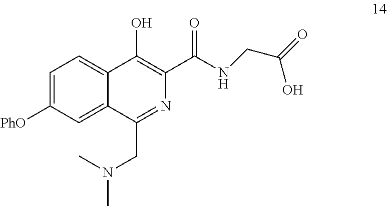 [SMILES: CN(C)Cc1nc(C(=O)NCC(=O)O)c(O)c2ccc(Oc3ccccc3)cc12]

In yet another embodiment the disclosure relates to a another process for preparation of Roxadustat comprising reacting the compound of formula (7′) preferably formula (7) with glycine to afford compound of formula (5′) preferably formula (5)

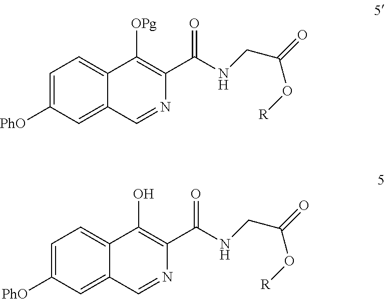

wherein R is H, substituted or unsubstituted alkyl, substituted or unsubstituted arylalkyl, or acid protecting group.

wherein Pg is H or —OH protecting group.

Compound (5′) preferably compound of formula (5) can be used as intermediate for the preparation of Roxadustat.

In yet another embodiment the disclosure relates to a another process for preparation of Roxadustat comprising formula (14); and conversion of formula (14) to compound (4) which can be later used for the preparation of Roxadustat.

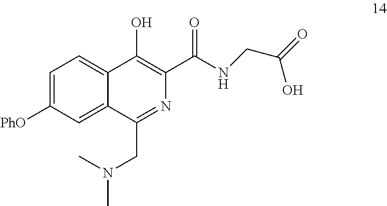 [SMILES: CN(C)Cc1nc(C(=O)NCC(=O)O)c(O)c2ccc(Oc3ccccc3)cc12]

In a preferred aspect, the present disclosure provides a process for preparation of Roxadustat comprising:

a) adding basic condition to the compound of formula (7′) preferably formula (7) to afford the compound of formula (6′) preferably formula (6).
b) coupling glycine ethyl ester with compound of formula (6′) preferably formula (6) to afford the compound of formula (5′) preferably formula (5).
c) alternatively, coupling glycine ester with compound of formula (7′) preferably formula (7) using acid conditions to afford the compound of formula (5′) preferably formula (5).
d) alternatively, combining the intermediate of formula (8) and formula (9) with halide of alkali and alakaline earth metals, preferably sodium iodide, potassium iodide, potassium bromide, preferably sodium iodide to afford the compound of formula (10), and conversion to formula (5′) preferably formula (5).
and;
e) coupling N,N,N′,N′-Tetramethylmethanediamine with the compound of formula (5) to afford the compound of formula (4).
f) alkylation of compound of formula (4) with methyl iodide, DMS or DMC to afford the compound of formula (3).
g) reduction of compound of formula (3) to afford the compound of formula (2), optionally with zinc or in the present of catalyst.
h) converting compound (2) to Roxadustat.

Optionally,

i) coupling compound (7) with glycine ethyl ester to afford compound (5′) preferably formula (5).
j) Coupling tetramethyldiaminomethane with compound (5′) preferably formula (5) to afford compound (14).
k) Coupling compound (14) with to obtain compound (4)
d) using compound (4) for the preparation of Roxadustat

compound of formula (5′) preferably formula (5) can be used following to the above process for the preparation of Roxadustat.

In another aspect Roxadustat is prepared according to the process describes herein

In further aspect Roxadustat prepared according to present disclosure is substantially pure.

The present disclosure also encompasses the use of the Roxadustat prepared by the processes of the present disclosure for the preparation of pharmaceutical compositions of Roxadustat.

The present disclosure comprises processes for preparing the above mentioned pharmaceutical compositions. The processes comprise combining the Roxadustat prepared by the processes of the present disclosure with at least one pharmaceutically acceptable excipient.

Roxadustat prepared by the processes of the present disclosure and the pharmaceutical compositions of Roxadustat prepared by the processes of the present disclosure can be used as medicaments, particularly for the treatment of anemia.

The present disclosure also provides methods for the treatment of anemia, comprising administering a therapeutically effective amount of Roxadustat prepared by the processes of the present disclosure, or at least one of the above pharmaceutical compositions, to a subject in need of the treatment.

The following process described herein can be summarized by the following scheme (Scheme E):

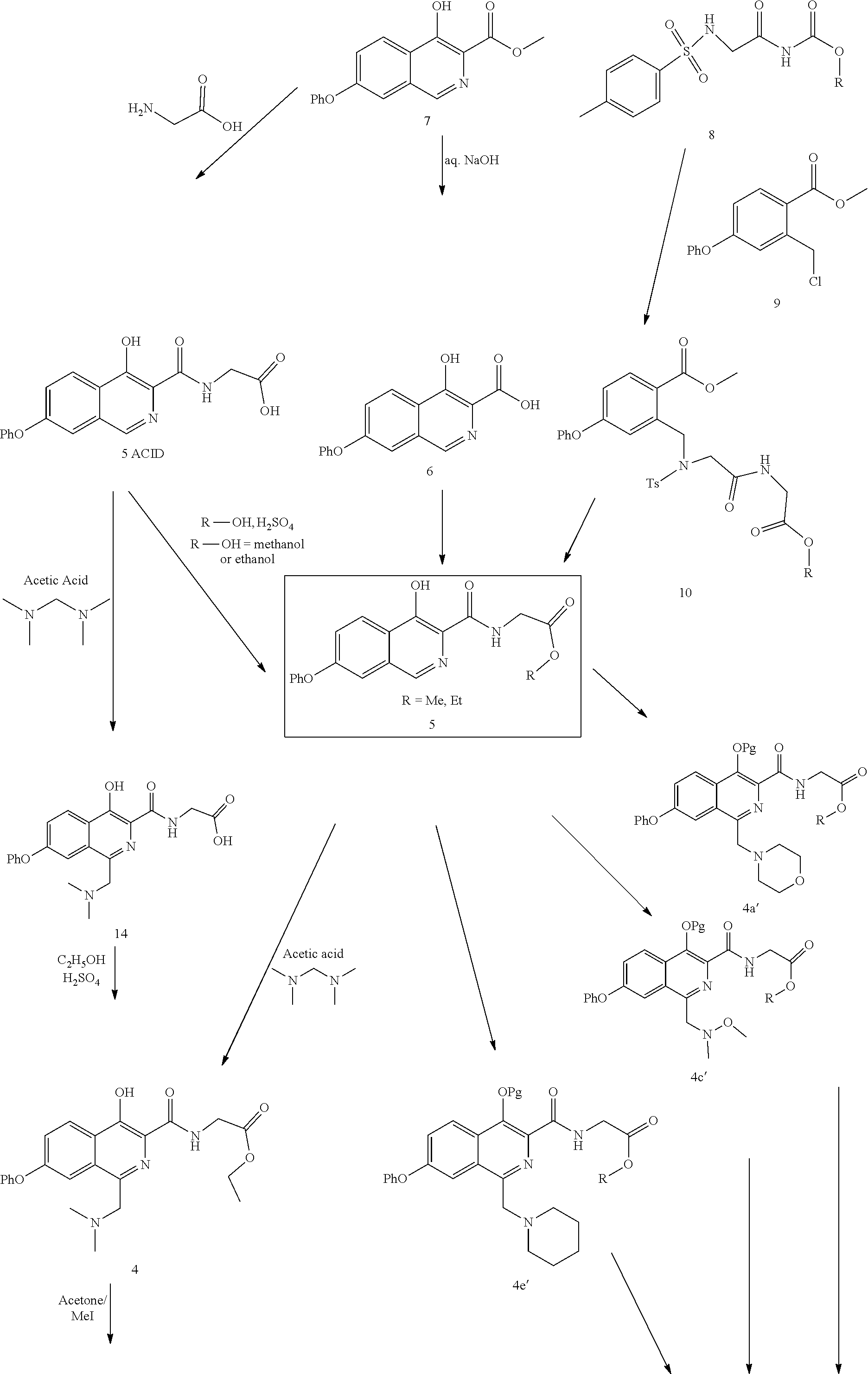

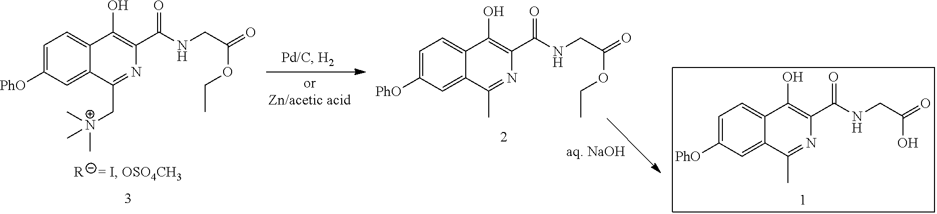

### DETAILED DESCRIPTION OF THE DISCLOSURE

The present disclosure provides new procedure and intermediates for the preparation of Roxadustat.

The present invention describes new process for providing Roxadustat in high yield and high purity that can be adapted to production in an industrial scale, i.e., greater than 1 kilogram scale.

Furthermore, this novel process uses a starting material that can lead only to one possible regioisomer, which confirms lack of regioselectivity problems. Moreover, the process of the present disclosure involves also inexpensive and safe reagents used for the synthesis. More specifically this novel process does not involve any acute hazardous material as well as explosive or genotoxic solvents or materials.

As defined herein, “high purity” refers to chemical purity wherein at least about more than 99%, more than about 99.5% or more than about 99.8% Roxadustat, measured by any technique described in the literature. e.g. by HPLC.

As used herein, and unless indicated otherwise, the term “isolated” in reference to the intermediates of the present disclosure, their solid state forms thereof corresponds to compounds that are physically separated from the reaction mixture in which they are formed.

A thing, e.g., a reaction mixture, may be characterized herein as being at, or allowed to come to “room temperature”, often abbreviated “RT.” This means that the temperature of the thing is close to, or the same as, that of the space, e.g., the room or fume hood, in which the thing is located. Typically, room temperature is from about 20° C. to about 30° C., or about 22° C. to about 27° C., or about 25° C.

The processes or steps may be referred to herein as being carried out “overnight.” This refers to time intervals, e.g., for the processes or steps, that span the time during the night, when the processes or steps may not be actively observed. The time intervals are from about 8 to about 20 hours, or about 10 to about 18 hours, or about 16 hours.

As used herein, and unless indicated otherwise, the term “reduced pressure” refers to a pressure of about 10 mbar to about 500 mbar, or about 50 mbar.

The amount of solvent employed in chemical processes, e.g., reactions or crystallizations, may be referred to herein as a number of “volumes” or “vol” or “V.” For example, a material may be referred to as being suspended in 10 volumes (or 10 vol or 10V) of a solvent. In this context, this expression would be understood to mean milliliters of the solvent per gram of the material being suspended, such that suspending a 5 grams of a material in 10 volumes of a solvent means that the solvent is used in an amount of 10 milliliters of the solvent per gram of the material that is being suspended or, in this example, 50 mL of the solvent. In another context, the term “v/v” may be used to indicate the number of volumes of a solvent that are added to a liquid mixture based on the volume of that mixture. For example, adding MTBE (1.5 v/v) to a 100 ml reaction mixture would indicate that 150 mL of MTBE was added.

As used herein, “halogen” or “halide” refers to fluoride, chloride, bromide or iodide. In certain embodiments, the halogen is bromide or iodide.

“Alkyl” refers to a monoradical of a branched or unbranched saturated hydrocarbon chain and can be substituted or unsubstituted. Lower alkyl groups may contain 1-6 carbon atoms or 1-4 carbon atoms. Examples of alkyl groups include, but are not limited to, methyl, ethyl, n-propyl, n-butyl, n-pentyl, n-hexyl, n-heptyl, n-octyl, n-nonyl, n-decyl, isopropyl, tert-butyl, isobutyl, etc.

“Alkoxy” refers to the O-(alkyl) group where the alkyl group is defined above.

“Aryl” refers to phenyl and 7-15 membered monoradical bicyclic or tricyclic hydrocarbon ring systems, including bridged, spiro, and/or fused ring systems, in which at least one of the rings is aromatic. Aryl groups can be substituted or unsubstituted. Examples include, but are not limited to, naphthyl, indanyl, 1,2,3,4-tetrahydronaphthalenyl, 6,7,8,9-tetrahydro-5H-benzocycloheptenyl, and 6,7,8,9-tetrahydro-5H-benzocycloheptenyl. An aryl group may contain 6 (i.e., phenyl) or 9 to 15 ring atoms, such as 6 (i.e., phenyl) or 9-11 ring atoms, e.g., 6 (i.e., phenyl), 9 or 10 ring atoms.

As defined herein, the intermediate/formula/compound described in this application have the following chemical names:

| 7 | R = methyl | 4-hydroxy-7-phenoxyisoquinoline-3-carboxylic acid methyl ester |
|---|---|---|
| 6 | | 4-hydroxy-7-phenoxyisoquinoline-3-carboxylic acid |
| 5 | R = ethyl | ethyl (4-hydroxy-7-phenoxyisoquinoline-3-carbonyl)glycinate |
| 5-methyl | | methyl (4-hydroxy-7-phenoxyisoquinoline-3-carbonyl)glycinate |
| 5-acid | | (4-hydroxy-7-phenoxyisoquinoline-3-carbonyl)glycine |
| 14 | | (1-((dimethylamino)methyl)-4-hydroxy-7-phenoxyisoquinoline-3- |
| | | carbonyl)glycine |
| 4 | R = ethyl | ethyl (1-((dimethylamino)methyl)-4-hydroxy-7-phenoxyisoquinoline-3- |
| | | carbonyl)glycinate |
| 3 | R = ethyl | 1-(3-((2-ethoxy-2-oxoethyl)carbamoyl)-4-hydroxy-7-phenoxyisoquinolin- |
| | | 1-yl)-N,N,N-trimethylmethanaminium iodide |
| 2 | R = ethyl | ethyl (4-hydroxy-1-methyl-7-phenoxyisoquinoline-3-carbonyl)glycinate |
| 1 | (Roxadustat) | (4-hydroxy-1-methyl-7-phenoxyisoquinoline-3-carbonyl)glycine |

| | |
|---|---|
| 8-methyl | methyl tosylglycylglycinate |
| 8-acid | Tosylglycylglycine |
| 9 | methyl 2-(chloromethyl)-4-phenoxybenzoate |
| 10-methyl | methyl 2-(((N-(2-((2-methoxy-2-oxoethyl)amino)-2-oxoethyl)-4- |
| | methylphenyl)sulfonamido)methyl)-4-phenoxybenzoate |
| 10-acid | N-(2-(methoxycarbonyl)-5-phenoxybenzyl)-N-tosylglycylglycine |
| 4a | methyl (4-hydroxy-1-(morpholinomethyl)-7-phenoxyisoquinoline-3- |
| | carbonyl)glycinate |
| 4b | (4-hydroxy-1-(morpholinomethyl)-7-phenoxyisoquinoline-3- |
| | carbonyl)glycine |
| 4c | methyl (4-hydroxy-1-((methoxy(methyl)amino)methyl)-7- |
| | phenoxyisoquinoline-3-carbonyl)glycinate |
| 4d | (4-hydroxy-1-((methoxy(methyl)amino)methyl)-7-phenoxyisoquinoline-3- |
| | carbonyl)glycine |
| 4e | methyl (4-hydroxy-7-phenoxy-1-(piperidin-1-ylmethyl)isoquinoline-3- |
| | carbonyl)glycinate |
| 4f | (4-hydroxy-7-phenoxy-1-(piperidin-1-ylmethyl)isoquinoline-3- |
| | carbonyl)glycine |

In another aspect, the present disclosure provides novel intermediates, their preparation and their use in the preparation of Roxadustat.

In another aspect, the present disclosure provides the novel compounds (3′), (4′) and (5′) preferably (3), (4) and (5).

In another aspect, the present disclosure provides the novel compounds (4a′), (4c′) and (4e′), preferably (4a), (4b), (4c), (4d), (4e) and (4f).

The present disclosure provides novel intermediates of formula (3′), (4′) and (5′) preferably (3), (4) and (5) and intermediates of formula (4a′), (4c′) and (4e′), preferably (4a), (4b), (4c), (4d), (4e) and (4f) that may advantageously be used for the preparation of Roxadustat

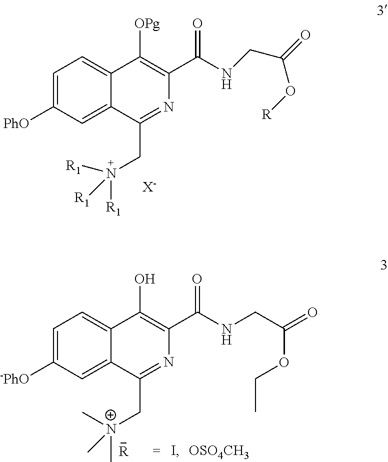

wherein R is selected from the list consisting of substituted or unsubstituted alkyl, substituted or unsubstituted arylalkyl, or acid protecting group. Preferably, R is selected from the group consisting of methyl, ethyl, t-butyl and benzyl, most preferably R is methyl.
wherein R¹ is H, substituted or unsubstituted alkyl, substituted or unsubstituted arylalkyl, or amine protecting group or one R¹ is alkyl and other R¹ is oxygen attached to alkyl group or hydrogen or two R¹ together with the nitrogen atom to which they are attached to form 4-8 membered heterocyclic like 1-Methylpiperazine, piperidine, pyrrolidine, morpholine or hetetroaromatic.
wherein X⁻ is anion of corresponding amine preferably Iodo or methanesulfate.
wherein Pg is H or —OH protecting group.

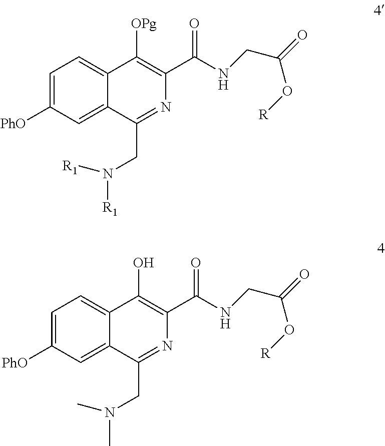

wherein R is H, substituted or unsubstituted alkyl, substituted or unsubstituted arylalkyl, or acid protecting group.
wherein R¹ is H, substituted or unsubstituted alkyl, substituted or unsubstituted arylalkyl, or amine protecting group or one R¹ is alkyl and other R¹ is oxygen attached to alkyl group or hydrogen or two R¹ together with the nitrogen atom to which they are attached to form 4-8 membered heterocyclic like 1-Methylpiperazine, piperidine, pyrrolidine, morpholine or hetetroaromatic.
wherein Pg is H or —OH protecting group.

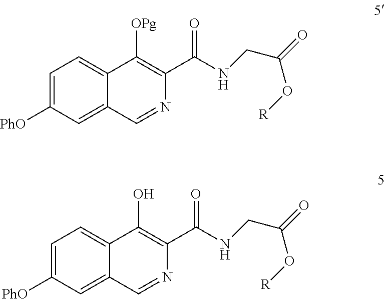

wherein R is H, substituted or unsubstituted alkyl, substituted or unsubstituted arylalkyl, or acid protecting group.
wherein Pg is H or —OH protecting group.

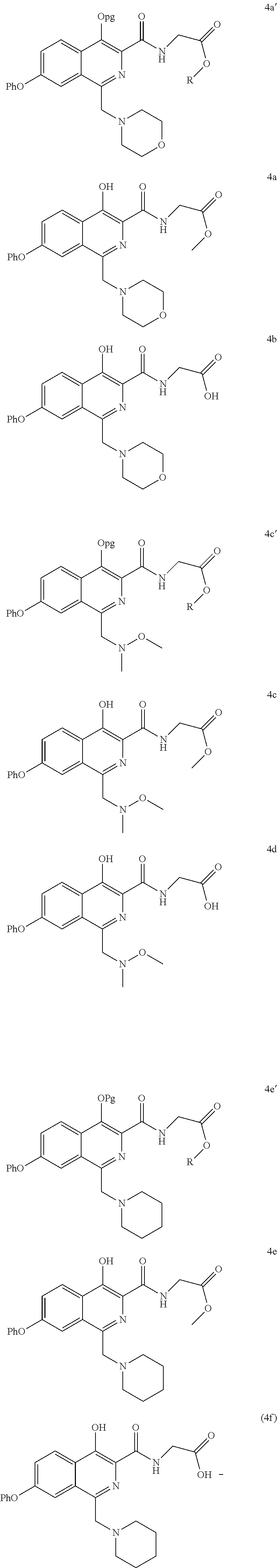

wherein R is selected from the list consisting of substituted or unsubstituted alkyl, substituted or unsubstituted arylalkyl, or acid protecting group preferably R is selected from the group consisting of methyl, ethyl, t-butyl and benzyl, most preferably R is ethyl or methyl.
wherein Pg is H or —OH protecting group.

In another aspect the present disclosure provides the use of any one of the compounds of formulae (3′), (4′) and (5′) preferably formula (3), (4), (5) and compound of formula (4a′), (4c′) and (4e′), preferably formula (4a), (4b), (4c), (4d), (4e) and (4f) in the preparation of Roxadustat.

In another aspect the present disclosure provides any one of compounds of formula (3′), (4′) and (5′) preferably formula (3), (4), (5) and compound of formula (4a′), (4c′) and (4e′), preferably formula (4a), (4b), (4c), (4d), (4e) and (4f) for use in the preparation of Roxadustat.

The present disclosure provides for novel processes for preparation of any one of compounds (3′), (4′) and (5′) preferably formula (3), (4), (5) and compound of formula (4a′), (4c′) and (4e′), preferably formula (4a), (4b), (4c), (4d), (4e) and (4f) for the preparation of Roxadustat.

In another aspect Roxadustat is prepared according to the process describes herein.

According yet to another aspect the present invention relates to novel process by using the intermediate of formula (7′) preferably formula (7) for the preparation of compound of formula (6′) preferably formula (6)

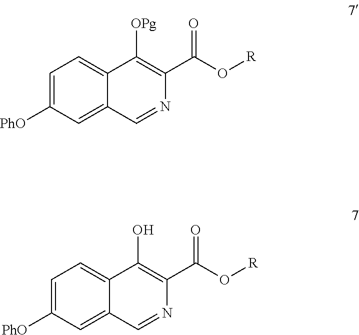

wherein R is H, substituted or unsubstituted alkyl, substituted or unsubstituted arylalkyl, or acid protecting group.
Wherein Pg is H or —OH protecting group.

According to one aspect the disclosure related to a process for preparation of compound of formula (6′) formula (6) by conversion of compound (7) via basic conditions to compound (6).

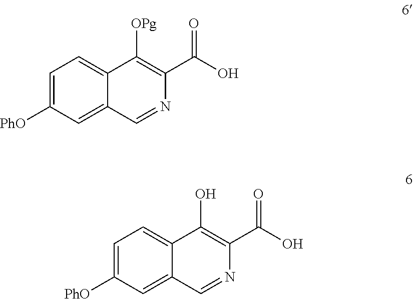

Wherein Pg is H or —OH protecting group.

The reaction may be performed in the presence of basic reagent such as sodium hydroxide.

According to one aspect the disclosure related to a process for preparation of compound of formula (5′) preferably formula (5) by condensation of formula (7′) preferably formula (7).

Preferably the condensation may be performed in the presence of an coupling reagent for example such as glycine ethyl ester

According yet to another aspect the present invention relates to novel process by using the intermediate of formula (7′) preferably formula (7) for the preparation of intermediate formula (5′) preferably formula (5) more preferably formula (5-acid) and formula (5-methyl)

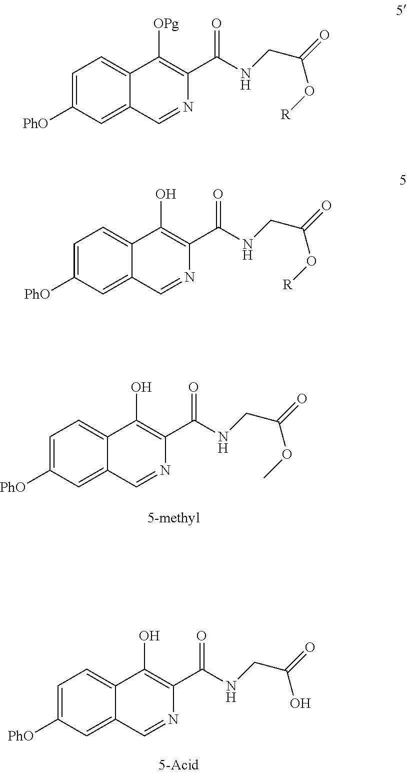

wherein R is H, substituted or unsubstituted alkyl, substituted or unsubstituted arylalkyl, or acid protecting group.
wherein Pg is H or —OH protecting group

According to one aspect the disclosure related to a process for preparation of compound of formula (5′) preferably formula (5) more preferably formula (5-ethyl) by coupling formula (7′) preferably compound (7) with Glycine ethyl ester.

The reaction may be performed in the presence of sodium methoxide.

According to yet another aspect the present invention relates to novel process by using the intermediate of formula (5-acid) for the preparation of intermediate formula (5-methyl).

In another embodiment the disclosure relates to a process for preparation of compound (5-methyl) comprising condensation of compound of formula (5-acid).

Preferably the condensation may be performed in the presence of a coupling reagent for example such as methanol under acid conditions, preferably sulfuric acid.

In another aspect the present disclosure provides novel intermediate of formula (5′) preferably formula (5) that can be used in the preparation of Roxadustat.

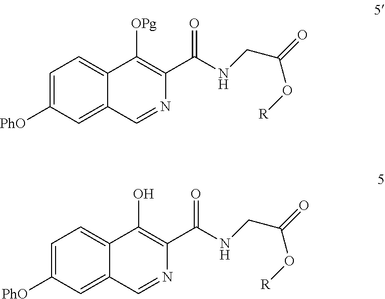

wherein R is selected from the list consisting of substituted or unsubstituted alkyl, substituted or unsubstituted arylalkyl, or acid protecting group preferably R is selected from the group consisting of methyl, ethyl, t-butyl and benzyl, most preferably R is methyl and ethyl.

wherein Pg is H or —OH protecting group.

In another aspect the present disclosure provides novel intermediate of formula (5′) preferably formula (5), and more preferably formula (5-methyl) and formula (5-acid) that can be used in the preparation of Roxadustat.

According to yet another aspect the present invention relates to novel process by using the intermediate of formula (6′) preferably formula (6) for the preparation of intermediate formula (5′) preferably formula (5).

In another embodiment the disclosure relates to a process for preparation of compound (5′) preferably compound (5) comprising condensation of compound of formula (6′) preferably formula (6).

Preferably the condensation may be performed in the presence of an coupling reagent for example such as glycine ethyl ester.

In another aspect the present disclosure provides another novel intermediate of formula (4′) preferably formula (4) that can be used in the preparation of Roxadustat.

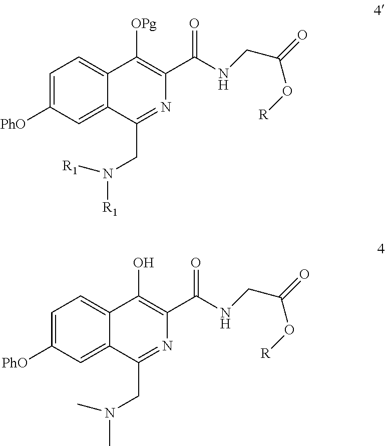

wherein R is selected from the list consisting of substituted or unsubstituted alkyl, substituted or unsubstituted arylalkyl, or acid protecting group preferably R is selected from the group consisting of methyl, ethyl, t-butyl and benzyl, most preferably R is methyl and ethyl.
wherein R¹ is H, substituted or unsubstituted alkyl, substituted or unsubstituted arylalkyl, or amine protecting group or one R¹ is alkyl and other R¹ is oxygen attached to alkyl group or hydrogen or two R¹ together with the nitrogen atom to which they are attached to form 4-8 membered heterocyclic like 1-Methylpiperazine, piperidine, pyrrolidine, morpholine or hetetroaromatic.
wherein Pg is H or —OH protecting group

According to yet another aspect the present invention relates to novel process by using the intermediate of formula (5′) preferably formula (5) for the preparation of intermediate formula (4′) preferably formula (4)

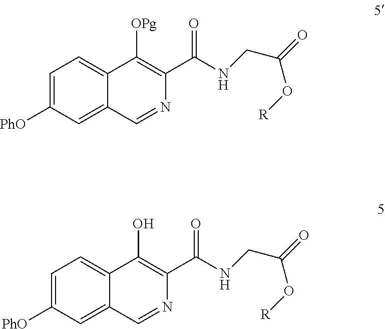

wherein R is selected from the list consisting of substituted or unsubstituted alkyl, substituted or unsubstituted arylalkyl, preferably R is selected from the group consisting of methyl, ethyl, t-butyl and benzyl, most preferably R is ethyl or methyl.
wherein Pg is H or —OH protecting group.

In yet another embodiment the disclosure relates to a process for preparation of compound of compound (4′) preferably formula (4) comprising coupling N,N,N′,N′-Tetramethylmethanediamine with compound (5).

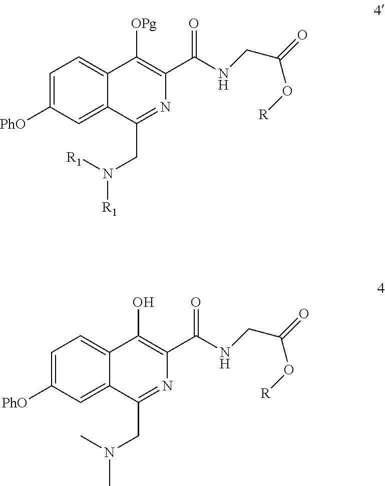

wherein R is H, substituted or unsubstituted alkyl, substituted or unsubstituted arylalkyl, or acid protecting group.

In another aspect the present disclosure provides another novel intermediate of formula (3′) preferably formula (3) that can be used in the preparation of Roxadustat.

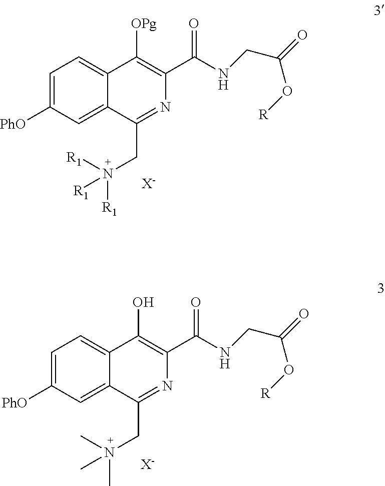

wherein R is selected from the list consisting of substituted or unsubstituted alkyl, substituted or unsubstituted arylalkyl, or acid protecting group preferably R is selected from the group consisting of methyl, ethyl, t-butyl and benzyl, most preferably R is methyl and ethyl.
wherein R¹ is H, substituted or unsubstituted alkyl, substituted or unsubstituted arylalkyl, or amine protecting group or one R¹ is alkyl and other R¹ is oxygen attached to alkyl group or hydrogen or two R¹ together with the nitrogen atom to which they are attached to form 4-8 membered heterocyclic like 1-Methylpiperazine, piperidine, pyrrolidine, morpholine or hetetroaromatic.
wherein X⁻ is anion of corresponding amine preferably Iodo or methanesulfate.
wherein Pg is H or —OH protecting group

According to yet another aspect the present invention relates to novel process by using the intermediate of formula (4′) preferably formula (4) for the preparation of intermediate formula (3′) preferably formula (3).

In yet another embodiment the disclosure relates to a process for preparation of compound of formula (3′) preferably formula (3) comprising reduction the compound of formula (4′) preferably formula (4) using reduction reagent, preferably methyl iodide and DMS. According to yet another aspect the present invention relates to novel process by using the intermediate of formula (3′) preferably formula (3) for the preparation of intermediate formula (2′) preferably formula (2)

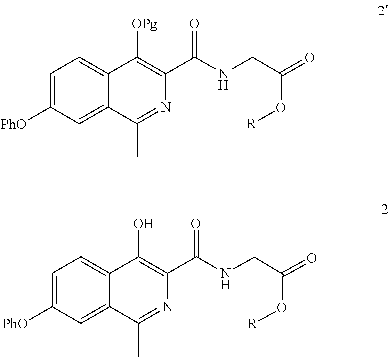

wherein R is H, substituted or unsubstituted alkyl, substituted or unsubstituted arylalkyl, or acid protecting group.
wherein Pg is H or —OH protecting group.

In yet another embodiment the disclosure relates to a process for preparation of compound of formula (2′) preferably formula (2) comprising reduction compound of formula (3′) preferably formula (3).

Preferably the reduction agent can be selected from zinc dust and palladium on carbon.

According to yet another aspect the present invention relates to novel process by using the intermediate of formula (2′) preferably formula (2) for the preparation Roxadustat.

In yet another aspect the present invention relates to novel process for Roxadustat by converting the intermediate of compound (2′) preferably compound (2) under basic conditions. Preferably with sodium hydroxide.

According to yet another aspect the present invention relates to novel process by using the intermediate of formula (2′) preferably formula (2) and more preferably formula (2-methyl) for the preparation of Roxadustat.

In yet another aspect the present invention relates to novel process for Roxadustat by converting the intermediate of formula (2′) preferably formula (2) and more preferably formula (2-methyl) under basic conditions, preferably with sodium hydroxide.

In yet further aspect the present disclosure provides novel intermediate of formula (5) preferably formula (5) that can be used in the preparation of formula (4′) preferably formula (4)

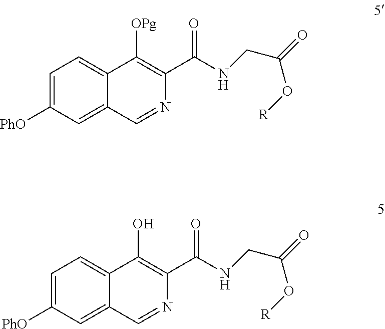

wherein R is selected from the list consisting of substituted or unsubstituted alkyl, substituted or unsubstituted aryl alkyl, or acid protecting group preferably R is selected from the group consisting of methyl, ethyl, t-butyl and benzyl, most preferably R is methyl or ethyl.
wherein Pg is H or —OH protecting group.

In yet further aspect the present disclosure provides novel intermediate of formula (4′) preferably formula (4) that can be used in the preparation of formula (3′) preferably formula (3)

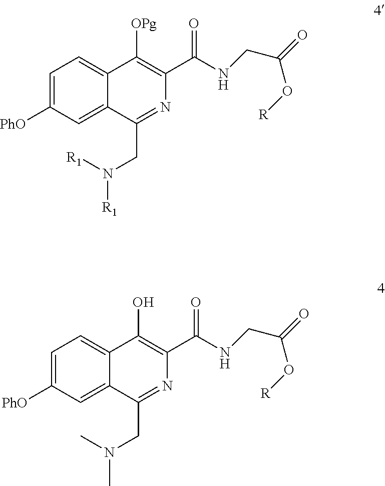

wherein R is selected from the list consisting of H, substituted or unsubstituted alkyl, substituted or unsubstituted arylalkyl or acid protecting group, preferably R is selected from the group consisting of H, methyl, ethyl, t-butyl and benzyl, most preferably R is methyl.
wherein R¹ is H, substituted or unsubstituted alkyl, substituted or unsubstituted arylalkyl, or amine protecting group or one R¹ is alkyl and other R¹ is oxygen attached to alkyl group or hydrogen or two R¹ together with the nitrogen atom to which they are attached to form 4-8 membered heterocyclic like 1-Methylpiperazine, piperidine, pyrrolidine, morpholine or hetetroaromatic.
wherein Pg is H or —OH protecting group

In yet further aspect the present disclosure provides novel intermediate of formula (3′) preferably formula (3) that can be used in the preparation of Roxadustat.

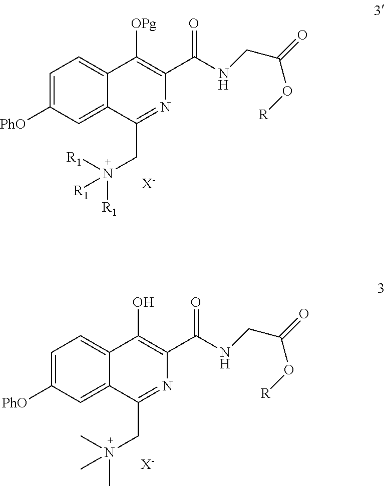

wherein R is selected from the list consisting of substituted or unsubstituted alkyl, substituted or unsubstituted arylalkyl, or acid protecting group. Preferably R is selected from the group consisting of methyl, ethyl, t-butyl and benzyl, most preferably R is methyl.
wherein R¹ is H, substituted or unsubstituted alkyl, substituted or unsubstituted arylalkyl, or amine protecting group or one R¹ is alkyl and other R¹ is oxygen attached to alkyl group or hydrogen or two R¹ together with the nitrogen atom to which they are attached to form 4-8 membered heterocyclic like 1-Methylpiperazine, piperidine, pyrrolidine, morpholine or hetetroaromatic. wherein X⁻ is anion of corresponding amine preferably Iodo or methanesulfate.
wherein Pg is H or —OH protecting group

In another aspect the present disclosure provides another novel intermediate of formula (4a′), (4c′) and formula (4e′) preferably formula (4a), (4b), (4c), (4d), (4e) and (4f) that can be used in the preparation of Roxadustat.

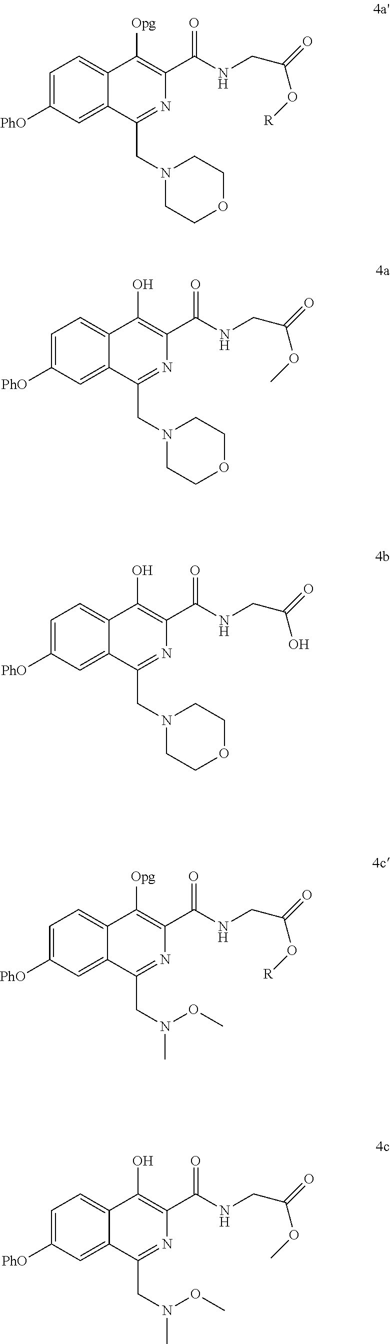

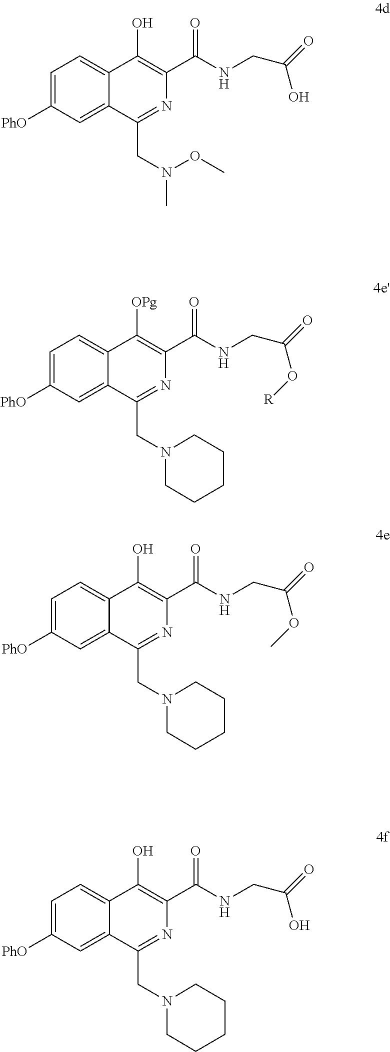

wherein R is H, substituted or unsubstituted alkyl, substituted or unsubstituted arylalkyl, or acid protecting group. Preferably methyl and H.
wherein Pg is H or —OH protecting group.

In yet further aspect the present disclosure provides novel intermediate of formula (5′) preferably formula (5) and more preferably (5-methyl) and preferably (5-acid) that can be used in the preparation of formula (4a′) preferably formula (4a) and (4b).

According to yet another aspect the present invention relates to novel process by using the intermediate of formula (5′) preferably formula (5) and more preferably formula (5-methyl) and formula (5-acid) for the preparation of intermediate of formula (4a′), (4c′) and formula (4e′) preferably formula (4a), (4b), (4c), (4d), (4e) and 4(f).

In yet another embodiment the disclosure relates to a process for preparation of compound of compound (4a′) preferably formula (4a) and (4b) comprising coupling morpholine with paraformaldehyde to form mannich base which reacts with formula (5′) preferably formula (5) and more preferably formula (5-methyl) and formula (5-acid) under acid conditions.

In yet further aspect the present disclosure provides novel intermediate of formula (4a′) preferably formula (4a) and (4b) that can be used in the preparation of formula (2′) preferably formula (2) and more preferably formula (2-methyl).

In yet further aspect the present disclosure provides novel intermediate of formula (4a′) preferably formula (4a) and (4b) that can be used in the preparation of Roxadustat.

Preferably, Roxadustat is obtained by reduction of formula (4a′) preferably formula (4a) and formula (4b) using reduction reagent selected from zinc, Sn, SnCl₂, Fe, Ni, and salt of this metal, Pd/C, Pt, Pd(OH)₂ and sodium/liquid ammonia, preferably with zinc under acid conditions.

According to yet another aspect the present invention relates to novel process by using the intermediate of formula (4a′) preferably formula (4a) and (4b) for the preparation of intermediate formula (2′) preferably formula (2) and more preferably formula (2-methyl).

In yet another embodiment the disclosure relates to a process for preparation of compound of formula (2′) preferably formula (2) and more preferably formula (2-methyl) comprising reduction the compound of formula (4a′) preferably formula (4a) and (4b) using reduction reagent selected from Sn, SnCl₂, Fe, Ni, and salt of this metal, Pd/C, Pt, Pd(OH)₂. Sodium/liquid ammonia and zinc, more preferably zinc under acid conditions.

Preferably reduction in the presence of Ethereal solvent, chlorinated solvent and dichloromethane, more preferably dichloromethane.

In yet further aspect the present disclosure provides novel intermediate of formula (4a′) preferably formula (4a) and (4b) that can be used in the preparation of formula (2′) preferably formula (2) and more preferably formula (2-methyl).

In yet further aspect the present disclosure provides novel intermediate of formula (4a′) preferably formula (4a) and (4b) that can be used in the preparation of Roxadustat.

Preferably, Roxadustat is obtained by reduction of formula (4a′) preferably formula (4a) and formula (4b) using reduction reagent selected from zinc, Sn, SnCl₂, Fe, Ni, and salt of this metal, Pd/C, Pt, Pd(OH)₂ and sodium/liquid ammonia, preferably with zinc under acid conditions. In yet another embodiment the disclosure relates to a process for preparation of compound of compound (4c′) preferably formula (4c) and (4d) comprising coupling N,O-Dimethylhydroxylamine with paraformaldehyde to form mannich base to react with compound (5-methyl) under acid conditions.

Preferably the coupling may be performed with paraformaldehyde, preferably using glacial acetic acid.

According to yet another aspect the present invention relates to novel process by using the intermediate of formula (4c′) preferably formula (4c) and (4d) for the preparation of intermediate formula (2′) preferably formula (2) and more preferably (2-methyl).

In yet another embodiment the disclosure relates to a process for preparation of compound of formula (2′) preferably formula (2) and more preferably formula (2-methyl) comprising reduction the compound of formula (4c′) preferably formula (4c) and (4d) using reduction reagent.

Preferably reduction with reduction reagents selected from zinc, Sn, SnCl₂, Fe, Ni, and salt of this metal, Pd/C, Pt, Pd(OH)₂. and Sodium/liquid ammonia. Preferably with zinc under acid conditions.

Preferably reduction in the presence of dichloromethane, Ethereal solvent, chlorinated alkane, acetic acid and water. More preferably, dichloromethane.

In yet further aspect the present disclosure provides novel intermediate of formula (2′) preferably formula (2) and more preferably formula (2-methyl) that can be used in the preparation of formula (4c′) preferably formula (4c) and (4d).

In yet further aspect the present disclosure provides novel intermediate of formula (4c′) preferably formula (4c) and (4d) that can be used in the preparation of formula (2′) preferably formula (2) and more preferably formula (2-methyl).

In yet further aspect the present disclosure provides novel intermediate of formula (4c′) preferably formula (4c) and (4d) that can be used in the preparation of Roxadustat.

Preferably Roxadustat is obtained by reduction of formula (4c′), preferably formula (4c) and (4d) with reduction reagents selected from zinc, Sn, SnCl₂, Fe, Ni, and salt of this metal, Pd/C, Pt, Pd(OH)₂ and Sodium/liquid ammonia, preferably with zinc under acid conditions. In yet another embodiment the disclosure relates to a process for preparation of compound of compound (4e′) preferably formula (4e) and (4f) comprising coupling piperidine with paraformaldehyde to form mannich base which reacts with formula (5′) preferably formula (5) and more preferably formula (5-methyl) and formula (5-acid) under acid conditions.

According to yet another aspect the present invention relates to novel process by using the intermediate of formula (4e′) preferably formula (4e) and (4f) for the preparation of intermediate formula (2′) preferably formula (2) and more preferably formula (2-methyl).

In yet another embodiment the disclosure relates to a process for preparation of compound of formula (2′) preferably formula (2) and more preferably formula (2-methyl) comprising reduction the compound of formula (4e′) preferably formula (4e) and (4f) using reduction reagent selected from Sn, SnCl₂, Fe, Ni, and salt of this metal, Pd/C, Pt, Pd(OH)₂. Sodium/liquid ammonia and zinc, more preferably zinc under acid conditions.

Preferably reduction in the presence of Ethereal solvent, chlorinated solvent and dichloromethane, more preferably dichloromethane.

In yet further aspect the present disclosure provides novel intermediate of formula (5′) preferably formula (5) and more preferably (5-methyl) and (5-acid) that can be used in the preparation of formula (4e′) preferably formula (4e) and (4f).

In yet further aspect the present disclosure provides novel intermediate of formula (4e′) preferably formula (4e) and (4f) that can be used in the preparation of formula (2′) preferably formula (2) and more preferably formula (2-methyl).

In yet further aspect the present disclosure provides novel intermediate of formula (4e′) preferably formula (4e) and (4f) that can be used in the preparation of Roxadustat.

Preferably, Roxadustat is obtained by reduction of formula (4e′) preferably formula (4e) and formula (4f) using reduction reagent selected from zinc, Sn, SnCl₂, Fe, Ni, and salt of this metal, Pd/C, Pt, Pd(OH)₂ and sodium/liquid ammonia, preferably with zinc under acid conditions.

According yet to another aspect, the present disclosure provides the novel compounds (3′), (4′), (5′), (4a′), (4c′) and (4e′), preferably (3), (4), (5), (4a), (4b), (4c), (4d), (4e) and (4f).

In another aspect the present disclosure provides the use of any one of the compounds of formula (3′), (4′), (5′), (4a′), (4c′) and (4e′), preferably (3), (4), (5), (4a), (4b), (4c), (4d), (4e) and (4f) in the preparation of Roxadustat.

In another aspect the present disclosure provides any one of compounds of formula (3′), (4′), (5′), (4a′), (4c′) and (4e′), preferably (3), (4), (5), (4a), (4b), (4c), (4d), (4e) and (4f) for use in the preparation of Roxadustat.

The present disclosure provides for novel processes for preparation of any one of compounds (3′), (4′), (5′), (4a′), (4c′) and (4e′), preferably (3), (4), (5), (4a), (4b), (4c), (4d), (4e) and (4f) for the preparation of Roxadustat.

In yet another embodiment the disclosure relates to novel process for preparation of Roxadustat comprising—

a. converting formula (7′) preferably formula (7)

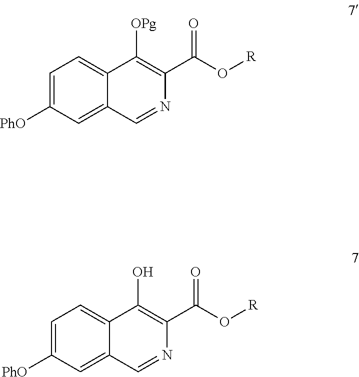

wherein R is H, substituted or unsubstituted alkyl, substituted or unsubstituted arylalkyl, or acid protecting group.
Wherein Pg is H or —OH protecting group.
To afford formula (6′) preferably formula (6)

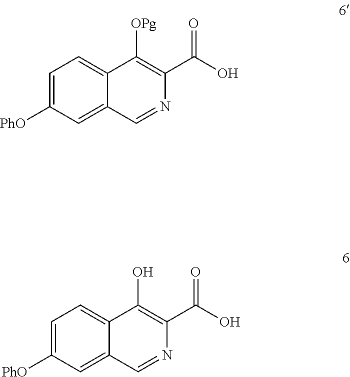

Wherein Pg is H or —OH protecting group;
and
b. condensation of formula (6′) preferably formula (6) to obtain the compound of formula (5′) preferably formula (5)

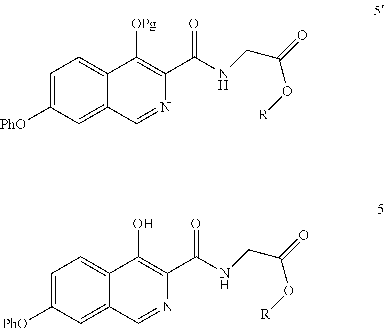

wherein R is H, substituted or unsubstituted alkyl, substituted or unsubstituted arylalkyl, or acid protecting group.
wherein Pg is H or —OH protecting group

### Optionally,

c. Condensation of formula (7′) preferably formula (7) to obtain the compound of formula (′5) preferably formula (5)
or
d. combining the intermediate of formula (8) and formula (9) to afford formula (10) followed by isoquinoline ring cyclisation to obtain the compound of formula (5′) preferably formula (5)

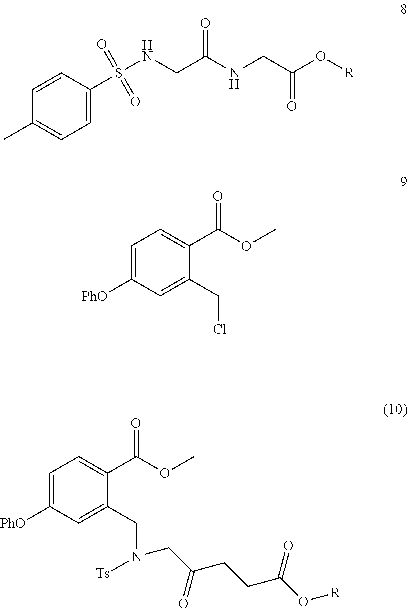

wherein R is H, substituted or unsubstituted alkyl, substituted or unsubstituted arylalkyl, or acid protecting group, preferably H or methyl.
and
e. reacting the compound of formula (5′) preferably formula (5) with N,N,N′,N′-Tetramethylmethanediamine to afford compound of formula 4

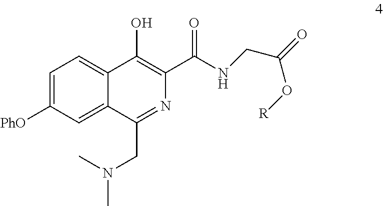

wherein R is H, substituted or unsubstituted alkyl, substituted or unsubstituted arylalkyl, or acid protecting group.
f. alkylation of compound 4 to afford compound of formula 3

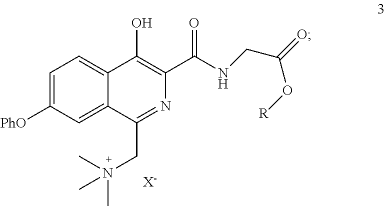

and
a. reduction the compound of formula 3, to afford formula 2

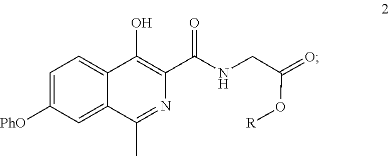

and
b. converting formula 2 to Roxadustat.

Preferably in step a) basic solution is used, more preferably sodium hydroxide

Step a) is typically carried out in the presence of one or more suitable protic or aprotic solvents or mixture thereof. Protic solvents selected from methanol, ethanol, propanol, isopropanol, water and aprotic solvent selected from THF, 2-methyl THF, acetone, ethyl acetate.

Preferably in steps b) and c) glycine ethyl ester or its salts such as hydrochloride is used.

Coupling in step b) and c) is typically with alkylamines selected from Tri ethyl amine, diisopropylethylamine, diisopropylamine, morpholine and propanephosphonic acid anhydride, or EDC, HOBt, DCC.

coupling formula (8) and formula (9) in step d) using halide of alkali and alkaline earth metals like sodium iodide, potassium iodide and potassium bromide more preferably sodium iodide to obtain formula (10)

preferably the solvent in the coupling step d) is dimethyl acetamide, dimethyl sulfoxide, N-methyl-2-pyrrolidone and preferably dimethylformamide.

preferably step d) using basic condition selected from carbonate, bicarbonate of alkali and alkaline earth metals, sodium carbonate, sodium bicarbonate, cesium carbonate, preferably sodium carbonate.

preferably in step d) isoquinoline ring cyclisation by alkoxide of alkali and alkaline earth metals, preferably sodium ethoxide, potassium tertiary butoxide, sodium tertiary butoxide, sodium amylate, potassium amylate preferably sodium methoxide.

Preferably isoquinoline ring cyclisation in step d) in alcoholic solvent, preferably methanol, ethanol, 2-propanol, butanol and preferably methanol.

Preferably in step e) acid solution is used preferably acetic acid.

Preferably in step f) the alkylating reagent can be selected from methyl iodide, Di methyl sulfate (DMS), Dimethyl carbonate (DMC). Preferably methyl iodide, Di methyl sulfate (DMS).

Preferably step f) is typically carried out in the presence of one or more suitable protic or aprotic solvents or mixture thereof Protic solvents selected from methanol, ethanol, propanol, isopropanol, water and aprotic solvent selected from THF, 2-methyl THF, acetone, ethyl acetate, preferably acetone.

Preferably in step g) zinc dust or palladium on carbon is used.

Preferably in step g) basic solution is used, more preferably sodium hydroxide.

In yet another embodiment the disclosure relates to novel process for preparation of Roxadustat comprising—a. coupling formula 7′, preferably formula 7 with Glycine to afford compound of formula (5′) preferably formula (5) more preferably formula (5-acid)

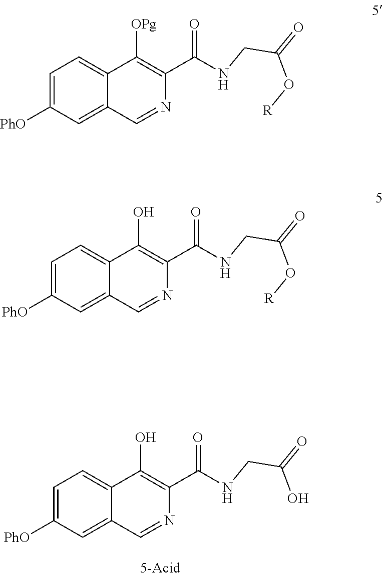

wherein R is H, substituted or unsubstituted alkyl, substituted or unsubstituted arylalkyl, or acid protecting group, preferably H or methyl
wherein Pg is H or —OH protecting group.

### Alternatively,

b. intermediate formula (5′) preferably formula (5) can alternatively be prepared by coupling the intermediate of formula (8) and formula (9) with halide of alkali and alkaline earth metals selected from sodium iodide, potassium iodide, potassium bromide, preferably sodium iodide under basic conditions to afford formula (10), followed by isoquinoline ring cyclisation with alkoxide of alkali and alkaline earth metals, preferably sodium methoxide, potassium tertiary butoxide, sodium tertiary butoxide, sodium amylate, potassium amylate, preferably sodium methoxide and;
c. reacting the compound of formula (5′) preferably (5) more preferably formula (5-methyl) and formula (5-acid) with morpholine and Paraformaldehyde under acid conditions, preferably glacial acetic acid to afford compound of formula (4a′) preferably formula (4a) and formula (4b).

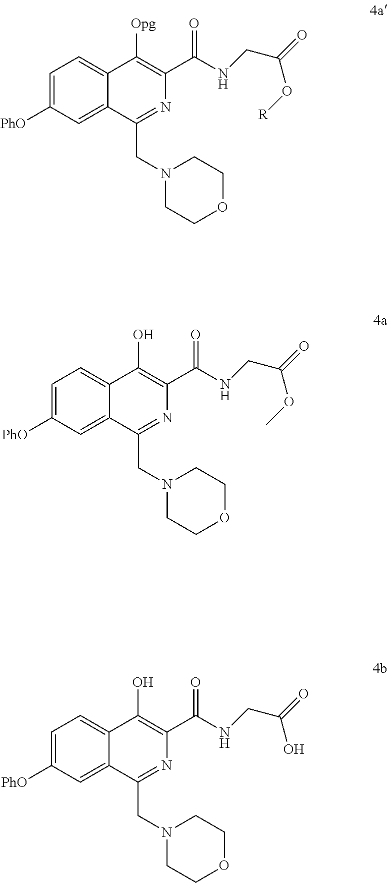

and;
d. reduction compound of formula (4a′) preferably formula (4a) and formula (4b) with reduction reagent selected from Sn, SnCl₂, Fe, Ni, and salt of this metal, Pd/C, Pt, Pd(OH)₂. Sodium/liquid ammonia and zinc, preferably with zinc under acid conditions, preferably with hydrochloric acid to afford formula (2′) preferably formula (2-methyl) and Roxadustat respectively.

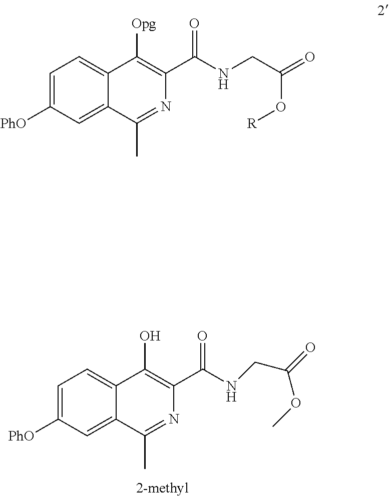

wherein R is H, substituted or unsubstituted alkyl, substituted or unsubstituted arylalkyl, or acid protecting group.
wherein Pg is H or —OH protecting group.
and
e. converting compound of formula (2′) preferably formula (2-methyl) to Roxadustat under basic conditions, preferably using sodium hydroxide.

In yet another embodiment the disclosure relates to novel process for preparation of Roxadustat comprising—

a. coupling formula 7′, preferably formula 7 with Glycine to afford compound of formula (5′) preferably formula (5) more preferably compound formula (5-acid)

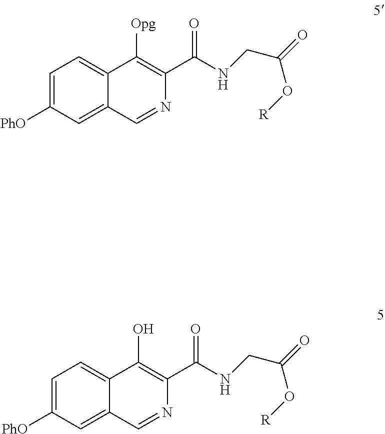

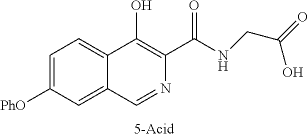 [SMILES: O=C(O)CNC(=O)c1ncc2cc(Oc3ccccc3)ccc2c1O]

alternatively,
b. Intermediate formula (5′) preferably formula (5) can alternatively be prepared by intermediate formula (5′) preferably formula (5) can alternatively be prepared by coupling the intermediate of formula (8) and formula (9) with halide of alkali and alkaline earth metals selected from sodium iodide, potassium iodide, potassium bromide, preferably sodium iodide under basic conditions to afford formula (10), followed by isoquinoline ring cyclisation with alkoxide of alkali and alkaline earth metals, preferably sodium methoxide, potassium tertiary butoxide, sodium tertiary butoxide, sodium amylate, potassium amylate, preferably sodium methoxide;
and
c. reacting the compound of formula (5′) preferably formula (5) more preferably formula (5-methy) and formula (5-acid) with N,O-Dimethylhydroxylamine and paraformaldehyde under acid conditions, preferably glacial acetic acid to afford compound of formula (4c′) and preferably formula (4c) and formula (4d)

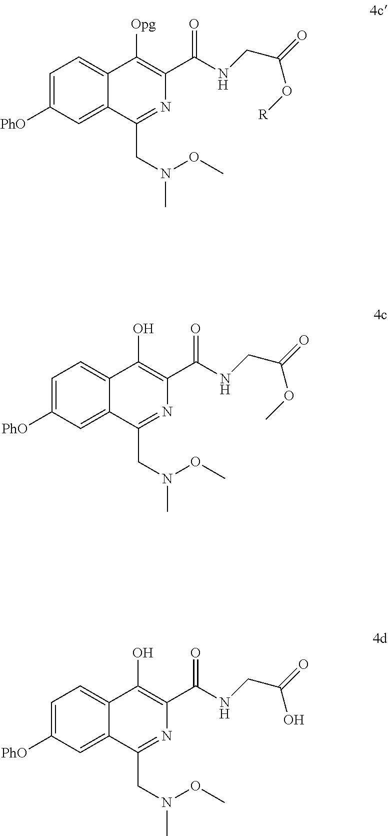

and;
d. reduction compound of formula (4c′) preferably formula (4c) and formula (4d) with reduction reagent selected from Sn, SnCl₂, Fe, Ni, and salt of this metal, Pd/C, Pt, Pd(OH)₂. Sodium/liquid ammonia and zinc preferably with zinc under acid conditions, preferably with hydrochloric acid to afford formula afford formula (2′) preferably formula (2-methyl) and Roxadustat respectively.

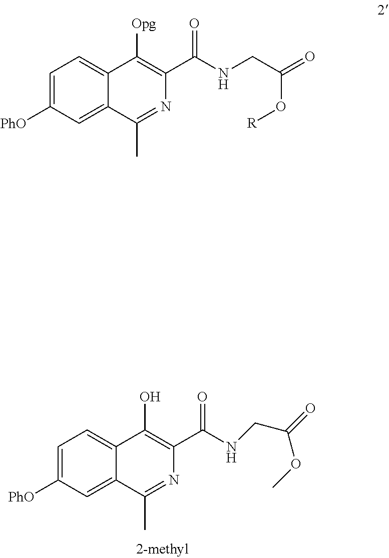

wherein R is H, substituted or unsubstituted alkyl, substituted or unsubstituted arylalkyl, or acid protecting group.
wherein Pg is H or —OH protecting group.
and;
e. converting compound of formula (2′) preferably formula (2-methyl) to Roxadustat under basic conditions, preferably using sodium hydroxide.

In yet another embodiment the disclosure relates to novel process for preparation of Roxadustat comprising—

a. coupling formula (7′), preferably formula (7) with Glycine to afford compound of formula (5′) preferably formula (5) and more preferably formula (5-acid)

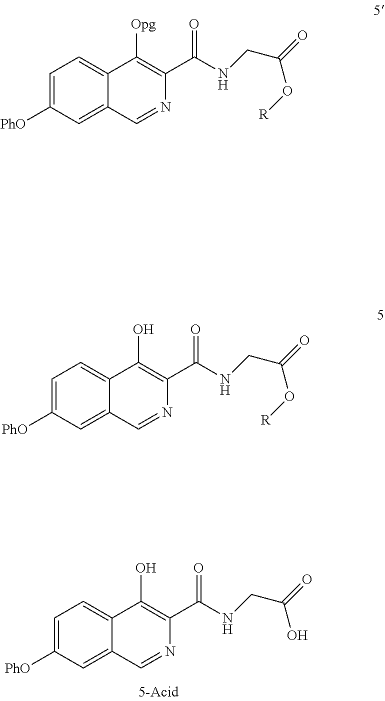

### Alternatively,

b. Intermediate formula (5′) preferably formula (5) can alternatively be prepared by coupling the intermediate of formula (8) and formula (9) with halide of alkali and alkaline earth metals selected from sodium iodide, potassium iodide, potassium bromide, preferably sodium iodide under basic conditions to afford formula (10), followed by isoquinoline ring cyclisation with alkoxide of alkali and alkaline earth metals, preferably sodium methoxide, potassium tertiary butoxide, sodium tertiary butoxide, sodium amylate, potassium amylate, preferably sodium methoxide and;
c. reacting the compound of formula (5′) preferably formula (5) more preferably formula (5-methyl) and formula (5-acid) with piperidine and paraformaldehyde under acid conditions, preferably glacial acetic acid to afford compound of formula (4e′) preferably formula (4e) and formula (4f)

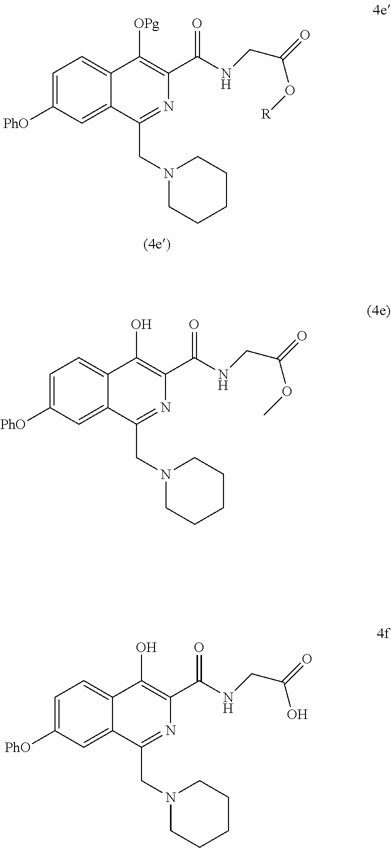

and;
d. reduction compound of formula (4e′) preferably formula (4e) and formula (4f) with reduction reagent selected from Sn, SnCl₂, Fe, Ni, and salt of this metal, Pd/C, Pt, Pd(OH)₂. Sodium/liquid ammonia and zinc preferably with zinc under acid conditions, preferably with hydrochloric acid to afford formula (2′) preferably formula (2-methyl) and Roxadustat respectively.

In another embodiment intermediate formula (5′) preferably formula (5) can be prepared by coupling the intermediate of formula (8) and formula (9) with halide of alkali and alkaline earth metals selected from sodium iodide, potassium iodide, potassium bromide, preferably sodium iodide under basic environment to afford formula (10), followed by isoquinoline ring cyclisation with alkoxide of alkali and alkaline earth metals, preferably sodium methoxide, potassium tertiary butoxide, sodium tertiary butoxide, sodium amylate, potassium amylate, preferably sodium methoxide

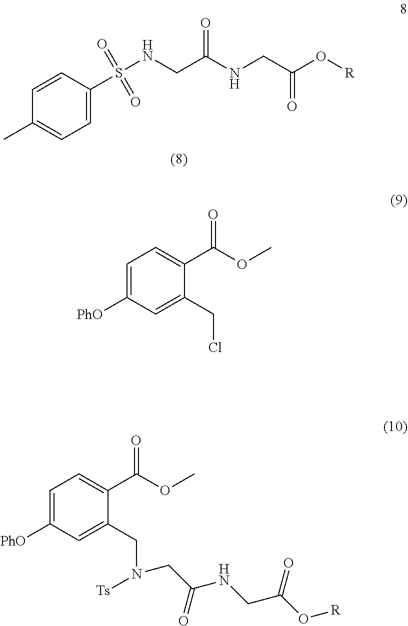

wherein R is selected from H, substituted or unsubstituted alkyl, substituted or unsubstituted arylalkyl, or acid protecting group, preferably H or methyl

According to yet another aspect the present invention relates to novel process by using the intermediate of formula (5′) preferably formula (5) for the preparation of formula (14)

According yet to another aspect the present invention relates to novel process for compound (14) by coupling formula (5′) preferably formula (5) with tetramethyldiaminomethane.

According to yet another aspect the present invention relates to novel process by using the intermediate of formula (14) for the preparation of intermediate formula (4) which can be later used for the preparation of Roxadustat.

According yet to another aspect the present invention relates to novel process for compound (4) using alcoholic solvent with acid. Preferably alcoholic solvent selected from methanol, ethanol. Acid is selected from sulfuric acid, paratoluenesulphonic acid and hydrochloric acid, preferable sulfuric acid.

In yet another embodiment the disclosure relates to a another process for preparation of Roxadustat comprising

a) reacting the compound of formula (7′) preferably formula (7) with glycine to afford compound of formula (5′) preferably formula (5)

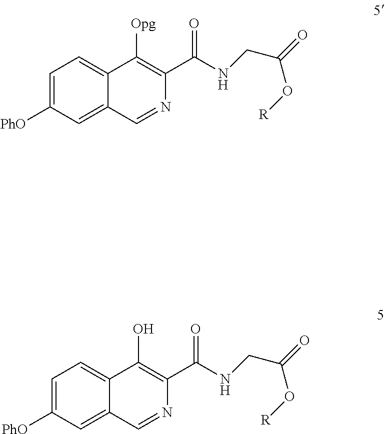

and;
b) adding tetramethyldiaminomethane and acetic acid to formula (5′) preferably formula (5) to afford compound of formula (14)

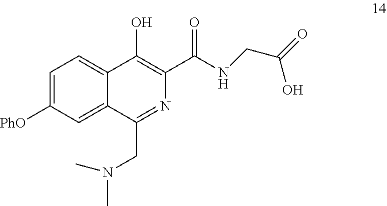 [SMILES: CN(C)Cc1nc(C(=O)NCC(=O)O)c(O)c2ccc(Oc3ccccc3)cc12]

and;
c) conversion of formula (14) to compound (4) with ethanol and sulfuric acid which can be later used for the preparation of Roxadustat.

Preferably in step a) sodium methoxide is used.

The disclosure may be further understood by the below reaction schemes (Schemes F, G and H), which are described in more detail below. These schemes form further aspect of the disclosure. Accordingly a further aspect of the disclosure provides processes for the preparation of Roxadustat or a salt thereof according to Scheme F:

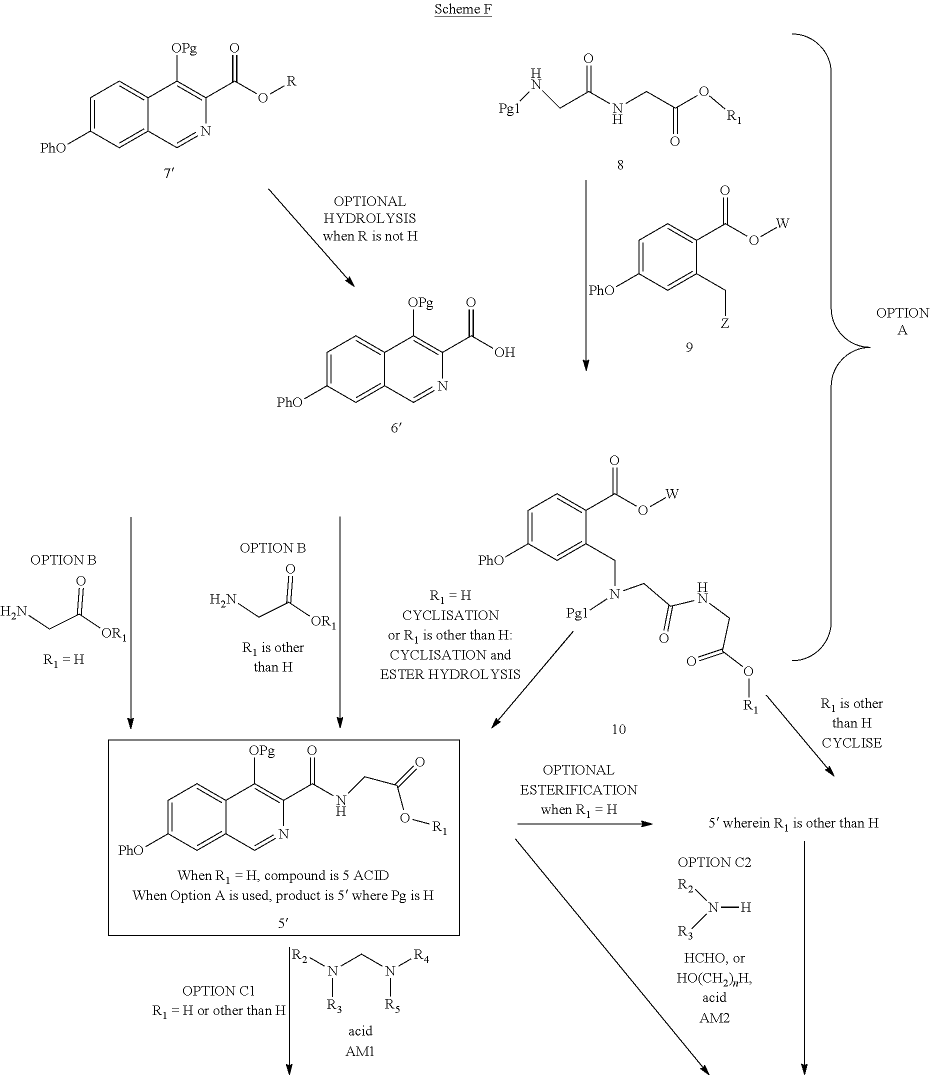

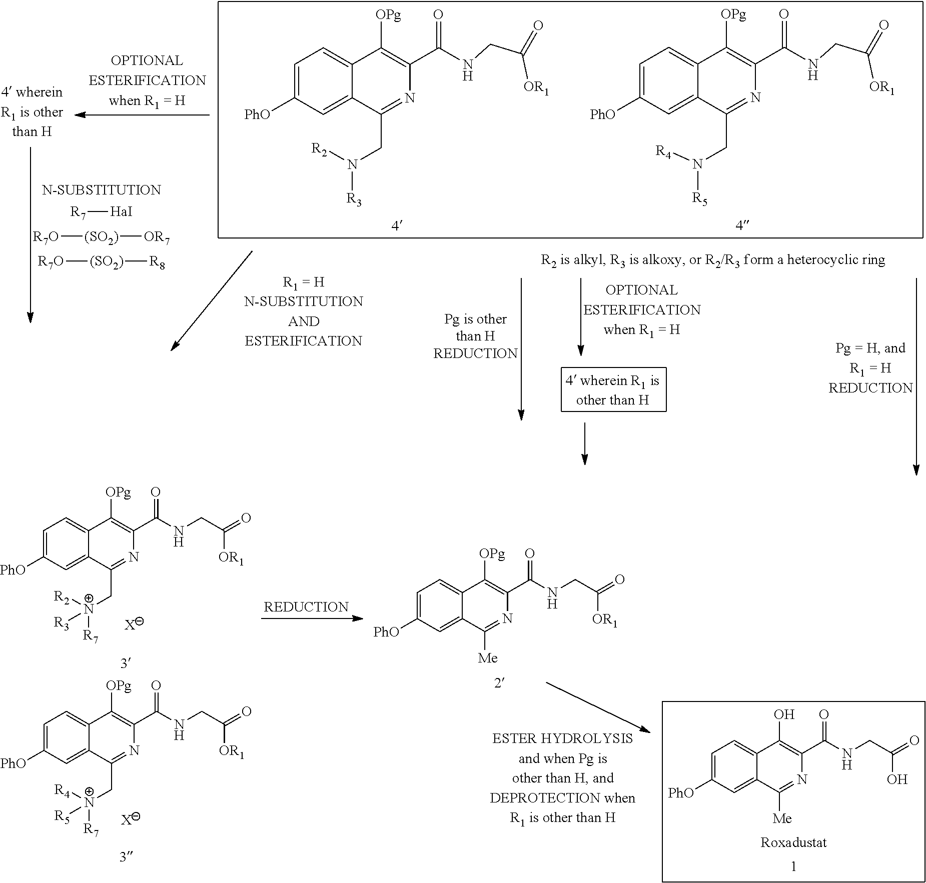

In Scheme F, AM1 in Option C1 for the preparation of compound 4′, 4″ or mixture thereof, may have R₂, R₃, R₄ and R₅ each independently being alkyl, arylalkyl or alkenyl, R₂ and R₃ and/or R₄ and R₅, taken together with the nitrogen atom to which they are bonded, each independently form a heterocyclic ring selected from:

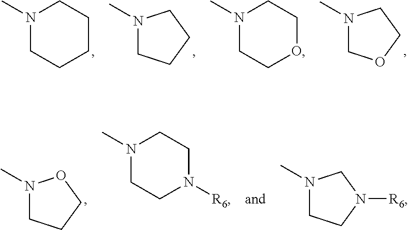

wherein R₆ is H or C₁₋₆ alkyl. In the above Scheme F, it will be appreciated that where AM1 is a symmetrical amine (i.e. R₄═R₂ and R₅═R₃), the resulting product is a compound of formula 4′, and likewise the product in the N-substitution/N-substitution/esterification step is a compound of formula 3. When AM1 is a non-symmetrical amine, the resulting product is a mixture of 4′ and 4″, and likewise the product in the N-substitution/N-substitution/esterification step is a mixture of 3′ and 3″. In any embodiment of the disclosed processes, AM1 is a symmetrical amine, such that the resulting compounds of formula 4′ and 3′ are not prepared as a mixture.

In the amine AM2 in option C2 for the preparation of compound 4′, R₂ is C₁₋₄ alkyl and R₃ is C₁₋₄ alkoxy; or R₂ and R₃ taken together with the nitrogen atom to which they are bonded, form a heterocyclic ring selected from:

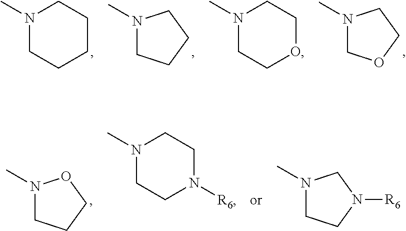

wherein R₆ is H or C₁₋₆ alkyl.

Also disclosed herein is a process for preparing Roxadustat or a salt thereof, wherein the compound of formula 4′ (see Scheme F) is prepared by Mannich reaction of the compound of formula 5′ wherein R₁ is H (i.e. 5 ACID) using an amine of formula AM2, formaldehyde or par4aformaldehyde, in the presence of an acid. Thus a further aspect of the disclosure provides a process preparing Roxadustat or salt thereof according to Scheme G:

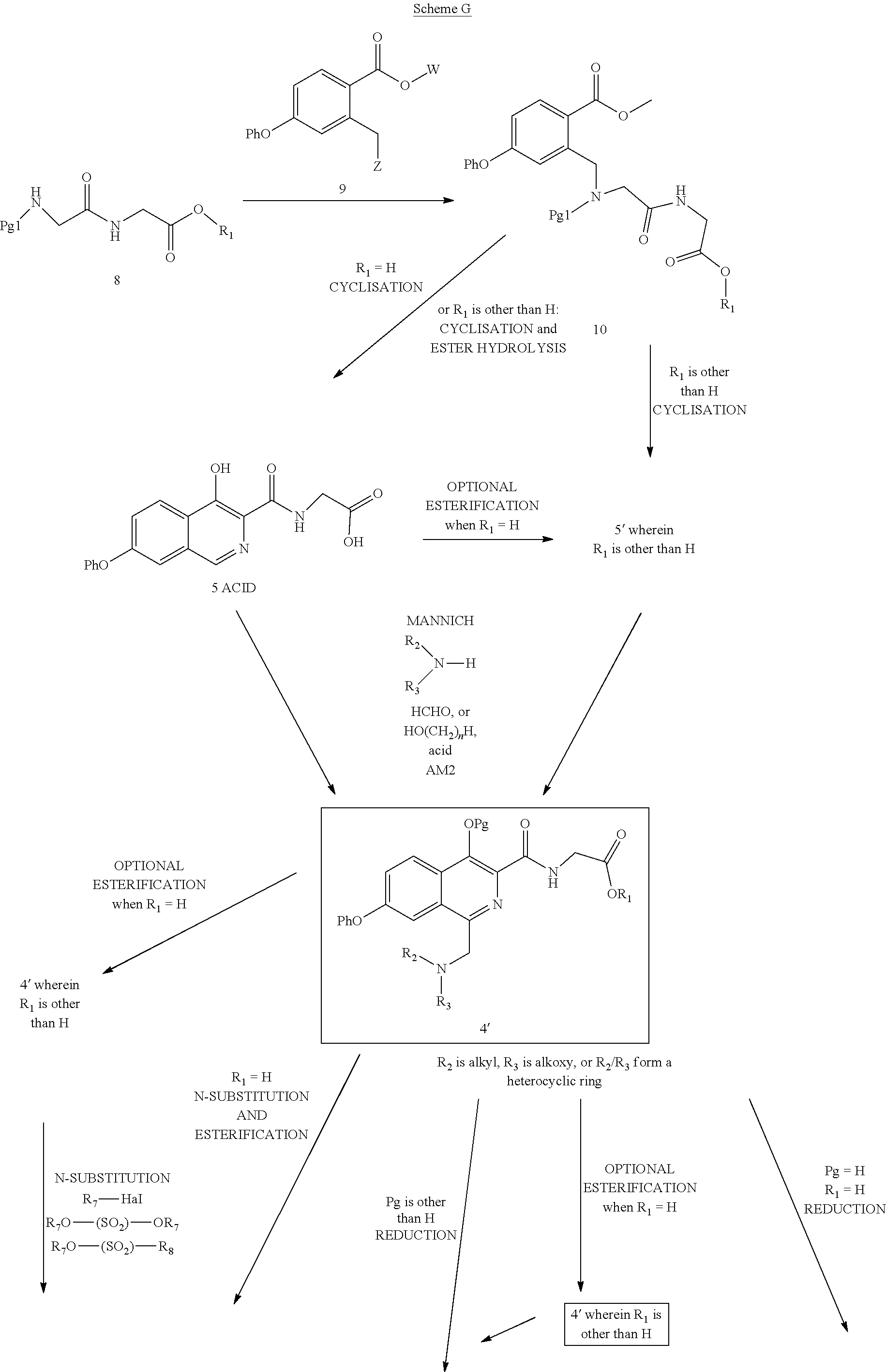

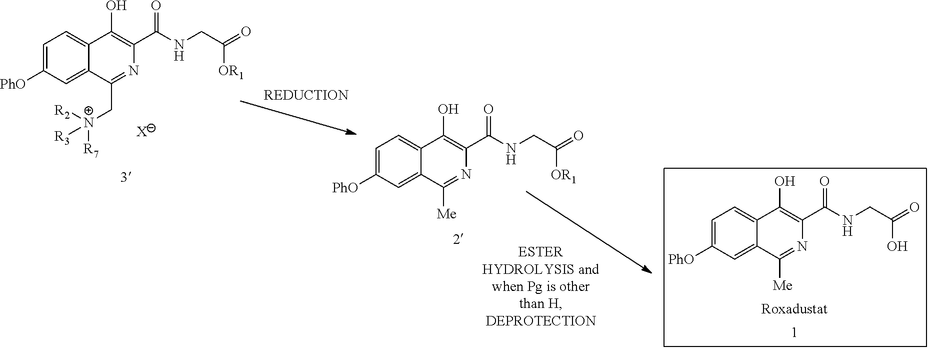

In Scheme G, the amine AM2 used in the preparation of compound 4′ from compound 5′ wherein R₁ is H (i.e. 5 ACID), has R₂ is C₁₋₄ alkyl and R₃ is C₁₋₄ alkoxy; or R₂ and R₃ taken together with the nitrogen atom to which they are bonded, form a heterocyclic ring selected from:

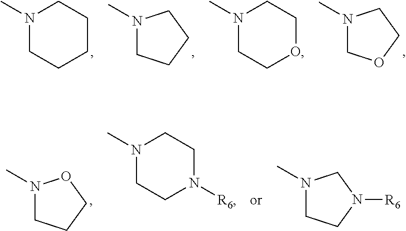

wherein R₆ is H or C₁₋₆ alkyl.

The resulting compound 4′ may be converted either by direct reduction (after any ester hydrolysis/Pg removal) to form Roxadustat, or may be converted to the compound 3′ before reduction (and removal of Pg/ester hydrolysis) to form Roxadustat. The direct reduction route for compound 4′ to Roxadustat is shown below in Scheme H. Scheme H therefore represent a further aspect of the present disclosure:

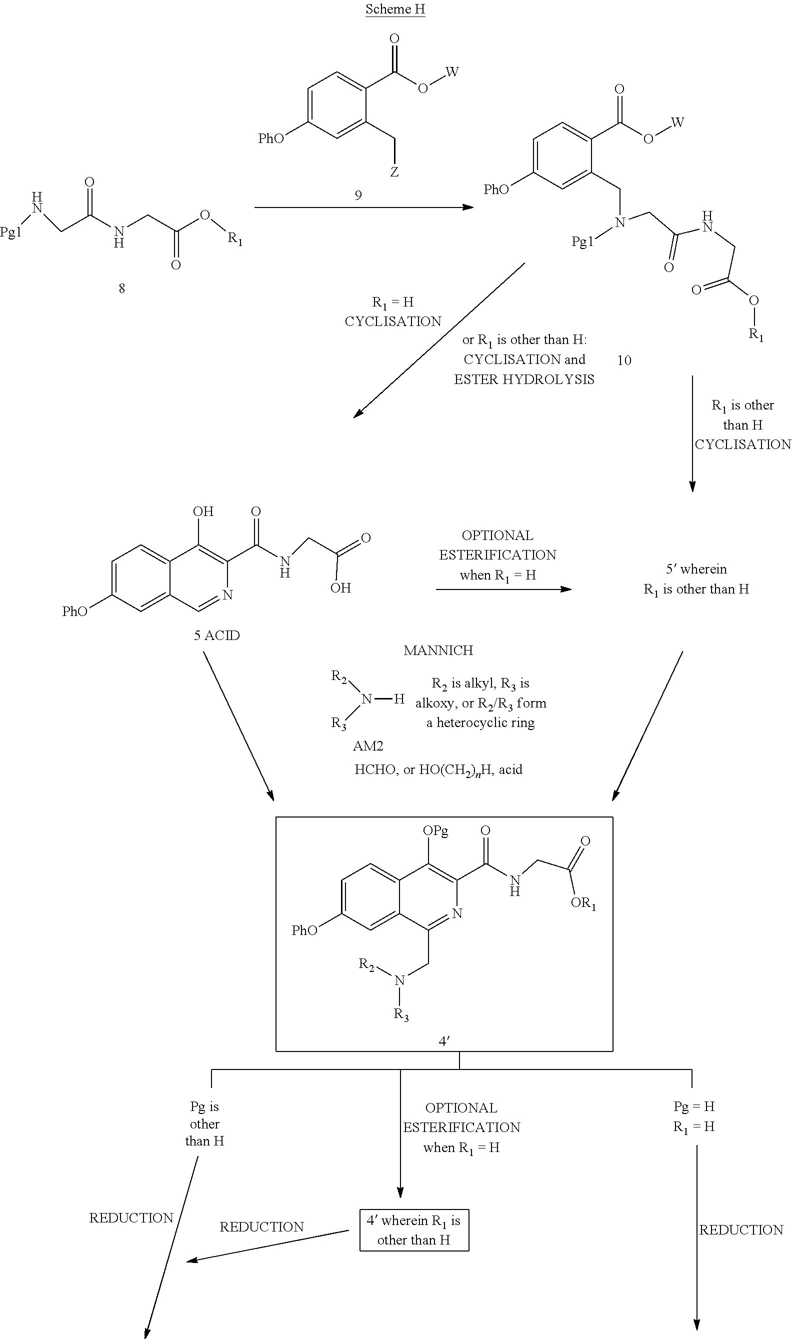

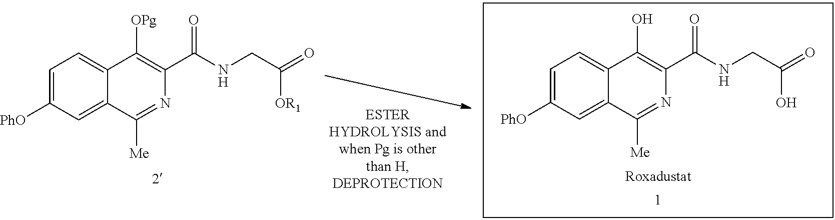

The Roxadustat may be converted to a salt by well-known procedures.

In any of the processes and compounds disclosed herein, including in the processes set out in Schemes F, G and H, unless otherwise indicated, the substituents may be defined as follows:
R is H, is alkyl, aryl, or arylalkyl or R is H, C₁₋₆ alkyl, C₆₋₁₀ aryl, or C₇₋₁₂ arylalkyl; or R is H or C₁₋₆ alkyl, or R is H or C₁₋₃ alkyl, or wherein R is H or methyl; and/or
Pg1 is a protecting group, optionally wherein Pg1 is —SO₂—Y, wherein Y is alkyl, aryl or alkylaryl; or wherein Y is C₁₋₆ alkyl, C₆₋₁₀ aryl, or C₇₋₁₂ alkylaryl; or wherein Y is methyl or tolyl, or wherein Y is para-tolyl; and/or
R₁ is H, alkyl, aryl, or arylalkyl, or wherein R₁ is H, C₁₋₆ alkyl, C₆₋₁₀ aryl, or C₇₋₁₂ arylalkyl; or R₁ is H, C₁₋₆ alkyl or C₁₋₃ alkyl, or wherein R₁ is H, methyl or ethyl, or wherein R₁ is H or methyl; and/or
Pg is H or a protecting group for hydroxy, wherein the protecting group may be an ester or ether OH protecting group; optionally wherein the protecting group Pg is methoxymethyl, tetrahydropyranyl, t-butyl, allyl, benzyl, benzyloxycarbonyl, t-butyldimethylsilyl, t-butyldiphenylsilyl; acetate, acetoxy, pivaloyl, or benzoyl; or wherein Pg is benzyl, benzyloxycarbonyl, or acetoxy; and/or
W is alkyl, aryl or arylalkyl, optionally wherein W is C₁₋₆ alkyl, optionally wherein W is methyl; and/or
Z is a leaving group, which is optionally halo, mesylate, tosylate, or besylate; or wherein Z is halo, optionally chloro, bromo or iodo; or optionally wherein Z is chloro; and/or
R₂, R₃, R₄, and R₅ each independently represents alkyl, arylalkyl or alkenyl; or R₂, R₃, R₄, and R₅ each independently represents C₁₋₆ alkyl, C₇₋₁₂ arylalkyl or C₂₋₆ alkenyl; or R₂, R₃, R₄, and R₅ each independently represents C₁₋₆ alkyl; optionally C₁₋₃ alkyl; or wherein each R₂, R₃, R₄, and R₅ are methyl; or wherein R₂ and R₃ and/or R₄ and R₅ taken together with the nitrogen atom to which they are bonded, each independently form a heterocyclic ring selected from:

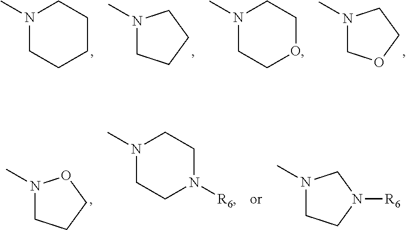

wherein R₆ is H or C₁₋₆ alkyl or optionally R₆ is H or methyl; or optionally, R₂ and R₃, and/or R₄ and R₅, taken together with the nitrogen atom to which they are bonded, each independently form a heterocyclic ring selected from:

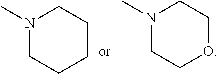

or R₂ and R₃ and/or R₄ and R₅, taken together with the nitrogen atom to which they are bonded, each independently form a heterocyclic ring selected from:

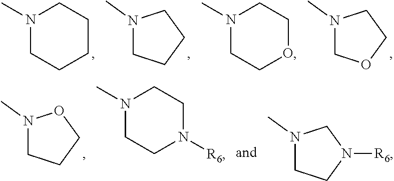

wherein R₆ is H or C₁₋₆ alkyl; R₇ is C₁ to C₆ alkyl; and/or
X⁻ is an anion selected from the group consisting of halide, O—SO₄—R₇ wherein R₇ is C₁ to C₆ alkyl, or O—SO₂—R₈ wherein R₈ is phenyl, tolyl, methyl or trifluoromethyl; or wherein X⁻ is an anion selected from the group consisting of chloro, bromo or iodo, or O—SO₄—R₇ wherein R₇ is C₁ to C₃ alkyl, and optionally wherein X⁻ is an iodo or O—SO₄-Me anion; and/or
R₈ phenyl, tolyl, methyl or trifluoromethyl.

The disclosure further provides the following processes set out in the numbered paragraphs below. These paragraphs represent further aspects and embodiments of the present disclosure:
1. A process for preparing Roxadustat or a salt thereof:

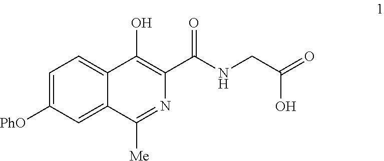 [SMILES: Cc1nc(C(=O)NCC(=O)O)c(O)c2ccc(Oc3ccccc3)cc12]

comprising:

(A) reducing a compound of formula 3′, 3″ or a mixture thereof:

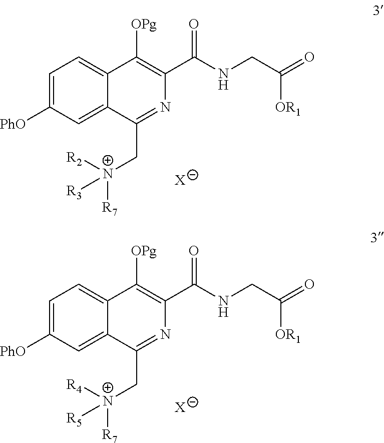

wherein Pg is H or a OH protecting group, R₁ is alkyl, aryl, or arylalkyl; R₂, R₃, R₄, and R₅ each independently represents alkyl, arylalkyl or alkenyl, or R₂ and R₃ and/or R₄ and R₅, taken together with the nitrogen atom to which they are bonded, each independently form a ring selected from:

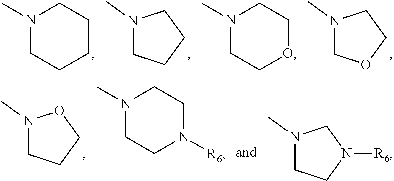

wherein R₆ is H or C₁₋₆ alkyl; R₇ is C₁ to C₆ alkyl and X⁻ is an anion selected from the group consisting of halide, O—SO₄—R₇ wherein R₇ is C₁ to C₆ alkyl, or O—SO₂—R₈ wherein R₈ is phenyl, tolyl, methyl or trifluoromethyl, to form a compound of formula 2′

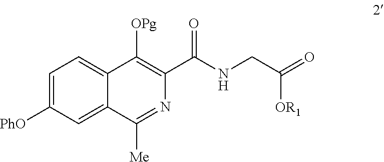

wherein Pg is H or an OH protecting group, R₁ is alkyl, aryl, or arylalkyl; and removing the R₁ group and where present removing the OH protecting group; or
   (B) reducing a compound of formula 4′, a compound of formula 4″ or a mixture thereof:

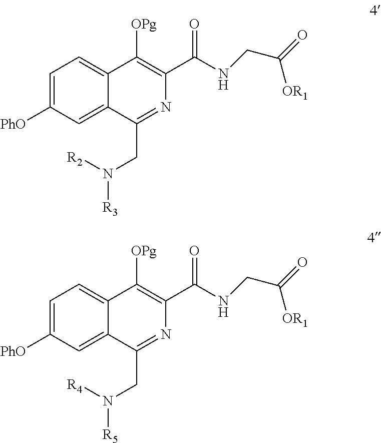

wherein Pg is H or an OH protecting group; R₁ is H, alkyl, aryl, or arylalkyl; R₂, R₃, R₄, and R₅ each independently represents alkyl, aryl, arylalkyl or alkenyl; or R₂ is C₁₋₄ alkyl and R₃ is C₁₋₄ alkoxy; or R₂ and R₃ and/or R₄ and R₅, taken together with the nitrogen atom to which they are bonded, each independently form a group selected from:

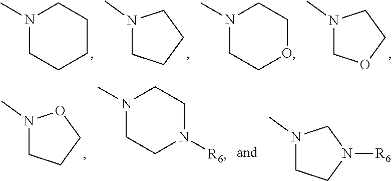

wherein R₆ is H or C₁₋₆ alkyl;
and where R₁ is not H, removing the R₁ group, and, where present, removing the OH protecting group.

Optionally, the compound 3′ is not present as a mixture with 3″.
2. Thus, also disclosed is a process according to paragraph 1 for preparing Roxadustat or a salt thereof, wherein (A) comprises reducing a compound of formula 3′:

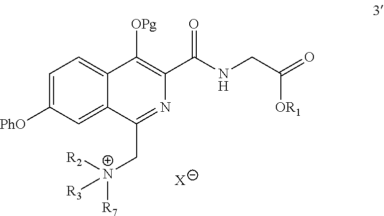

wherein Pg is H or a OH protecting group, R₁ is alkyl, aryl, or arylalkyl; R₂, R₃, each independently represents alkyl, arylalkyl or alkenyl, or R₂ and R₃, taken together with the nitrogen atom to which they are bonded, each independently form a ring selected from:

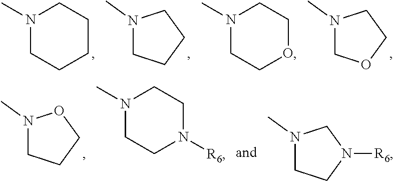

wherein R₆ is H or C₁₋₆ alkyl; R₇ is C₁ to C₆ alkyl and X⁻ is an anion selected from the group consisting of halide, O—SO₄—R₇, or O—SO₂—R₈ wherein R₈ is phenyl, tolyl, methyl or trifluoromethyl.
3. A process according to paragraph 2, wherein the reduction is carried out using:
   zinc and an acid, optionally zinc and acetic acid; or
   hydrogen gas and Pd/catalyst. Optionally the reduction is carried out using zinc and acetic acid.
4. A process according to paragraph 3, wherein the reduction is carried out in a polar solvent, wherein the polar solvent is optionally an alcohol, a ketone or an ester, and optionally a ketone, or wherein the polar solvent is acetone.
   In the process of paragraph 1, optionally, the compound 4′ is not present as a mixture with 4″.
5. Thus, also disclosed is a process according to paragraph 1, wherein (B) comprises reducing a compound of formula 4′:

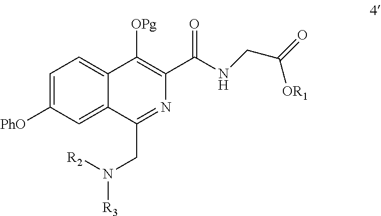

wherein Pg is H or an OH protecting group; R₂ is C₁₋₄ alkyl and R₃ is C₁₋₄ alkoxy; or R₂, R₃, R₄, and R₅ each independently represents alkyl, aryl, arylalkyl or alkenyl; or R₂ and R₃ and/or R₄ and R₅, taken together with the nitrogen atom to which they are bonded, each independently form a group selected from:

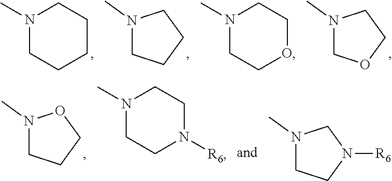

wherein R₆ is H or C₁₋₆ alkyl.
6. A process according to any of paragraphs 1, 2, 3, 4 or 5, wherein Pg is H or an ester or ether OH protecting group.
7. A process according to paragraph 6, wherein Pg is H, methoxymethyl, tetrahydropyranyl, t-butyl, allyl, benzyl, benzyloxycarbonyl, t-butyldimethylsilyl, t-butyldiphenylsilyl; acetate, acetoxy, pivaloyl, or benzoyl.
8. A process according to paragraph 7, wherein Pg is H, benzyl, benzyloxycarbonyl, or acetoxy.
9. A process according to paragraph 7, wherein Pg is H.
10. A process according to any of paragraphs 1, 2, 3, 4, 5, 6, 7, 8, or 9, wherein R₁ is H, C₁₋₆ alkyl, C₆₋₁₀ aryl, or C₇₋₁₂ arylalkyl.
11. A process according to paragraph 10, wherein R₁ is H or C₁₋₄ alkyl.
12. A process according to paragraph 10, wherein R₁ is H or methyl.
13. A process according to any of paragraphs 1, 2, 3, 4, 5, 6, 7, 8, 9, 10, 11, or 12, wherein Pg is H and R₁ is H or C₁₋₄ alkyl, or wherein Pg is H and R₁ is H, methyl or ethyl, or wherein Pg is H and R₁ is H or methyl.
14. A process according to any of paragraphs 2, 3, 4, 5, 6, 7, 8, 9, 10, 11, 12, or 12, wherein R₂ and R₃ each independently represents C₁₋₆ alkyl, C₇₋₁₂ arylalkyl or C₂₋₆ alkenyl.
15. A process according to any of paragraphs 2, 3, 4, 5, 6, 7, 8, 9, 10, 11, 12, 13 or 14, R₂, and R₃ each independently represents C₁₋₆ alkyl, C₁₋₃ alkyl, or R₂ and R₃ are each methyl.
16. A process according to any of paragraphs 5, 6, 7, 8, 9, 10, 11, 12, 13, 14, or 15, wherein the reduction is carried out using:
   zinc and an acid, optionally zinc and hydrochloric acid, or zinc and concentrated hydrochloric acid.
17. A process according to any of paragraphs 5, 6, 7, 8, 9, 10, 11, 12, 13, 14, or 15, wherein the reduction is carried out using hydrogen gas and Pd/catalyst.
18. A process according to any of paragraphs 1, 2, 3, 4, 5, 6, 7, 8, 9, 10, 11, 12, 13, 14, 15, 16, or 17, wherein the R₁ group is removed by hydrolysis, wherein the hydrolysis is carried out using a base, and optionally one or more polar solvents.
19. A process according to paragraph 18, wherein the base is an alkaline metal hydroxide, optionally sodium hydroxide or potassium hydroxide, and optionally sodium hydroxide.
20. A process according to paragraph 18 or paragraph 19, wherein the one or more polar solvents is selected from the group consisting of an ether, a ketone, an alcohol, or water, optionally wherein the solvent comprises a cyclic ether, an aliphatic alcohol and water, or wherein the solvent comprises THF, ethanol and water.
21. A process according to paragraphs 1, 2, 3, or 4, wherein the compound of formula 3′, 3″ or mixture thereof:

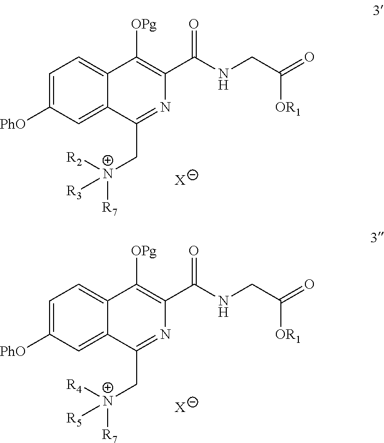

is prepared by a process comprising reacting a compound of formula 4′, a compound of formula 4″ or a mixture thereof:

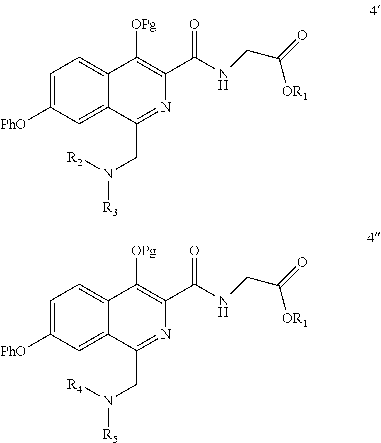

wherein Pg is H or an OH protecting group, R₁ is H, alkyl, aryl, or arylalkyl; R₂, R₃, R₄, and R₅ each independently represents alkyl, arylalkyl or alkenyl; or R₂ and R₃ and/or R₄ and R₅, taken together with the nitrogen atom to which they are bonded, each independently form a ring selected from:

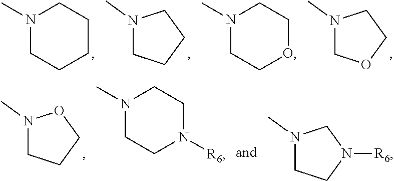

wherein R₆ is H or C₁₋₆ alkyl;
with:
   a compound R₇-Hal, wherein R₇ is C₁ to C₆ alkyl and Hal is halo;
   a compound of formula R₇O—(SO₂)—OR₇ wherein R₇ is C₁ to C₆ alkyl; or
   a compound of formula R₇O—(SO₂)—R₈, wherein R₇ is C₁ to C₆ alkyl and R₈ is phenyl, tolyl, methyl or trifluoromethyl;
wherein when R₁ in the compound 4′ or 4″ is H, the resulting compound 3′, compound 3″ or a mixture thereof, has R₁═R₇.
   Optionally, the compound of formula 4′ is not present as a mixture with the compound of formula 4″.
22. Accordingly, disclosed is a process according to paragraph 21 wherein the compound of formula 3′

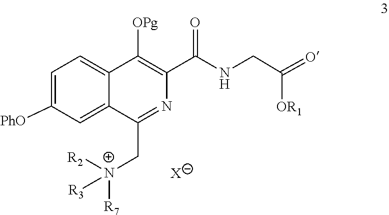

is prepared by a process comprising reacting a compound of formula 4′:

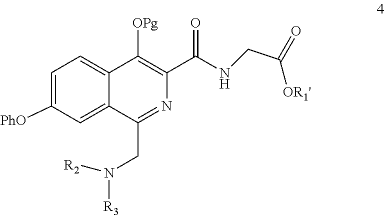

wherein Pg is H or an OH protecting group, R₁ is H, alkyl, aryl, or arylalkyl; R₂ and R₃, each independently represents alkyl, arylalkyl or alkenyl; or R₂ and R₃ taken together with the nitrogen atom to which they are bonded, each independently form a ring selected from:

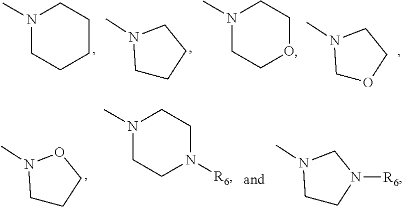

wherein R₆ is H or C₁₋₆ alkyl;
with:
   a compound R₇-Hal, wherein R₇ is C₁ to C₆ alkyl and Hal is halo; or
   a compound of formula R₇O—(SO₂)—OR₇ wherein R₇ is C₁ to C₆ alkyl; or
   a compound of formula R₇O—(SO₂)—R₈, wherein R₇ is C₁ to C₆ alkyl and R₈ is phenyl, tolyl, methyl or trifluoromethyl;
wherein when R₁ in the compound 4′ is H, the resulting compound 3′ has R₁═R₇.
23. A process according to paragraph 21 or paragraph 22, wherein R₇ is C₁ to C₃ alkyl; and optionally methyl.
24. A process according to any of paragraphs 21, 22 or 23, wherein X⁻ is an anion selected from the group consisting of chloro, bromo or iodo, or O—SO₄—R₇ wherein R₇ is C₁ to C₃ alkyl, and optionally wherein X⁻ is iodo or O—SO₄-Me.
25. A process according to any of paragraphs 21, 22, 23, or 24, wherein the compound R₇-Hal, is a C₁ to C₃ alkyl halide, or methyl halide, or methyl iodide.
26. A process according to any of paragraphs 21, 22, 23, or 24, wherein the compound of formula R₇O—(SO₂)—OR₇ is a di(C₁₋₃ alkyl)sulfate, or dimethylsulfate.
27. A process according to any of paragraphs 21, 22, 23, 24, 25 or 26, wherein the reaction is carried out in an aprotic solvent, optionally wherein the aprotic solvent is a ketone, a nitrile or an ester, optionally wherein the aprotic solvent is acetone, acetonitrile, or ethylacetate, or wherein the reaction is carried out in acetone.
28. A process according to any of paragraphs 1, 5, 6, 7, 8, 9, 10, 11, 12, 13, 14, 15, 16, 17, 18, 19, 20, 21, 22, 23, 24, 25, 26 or 27, wherein the compound of formula 4′, 4″ or mixture thereof:

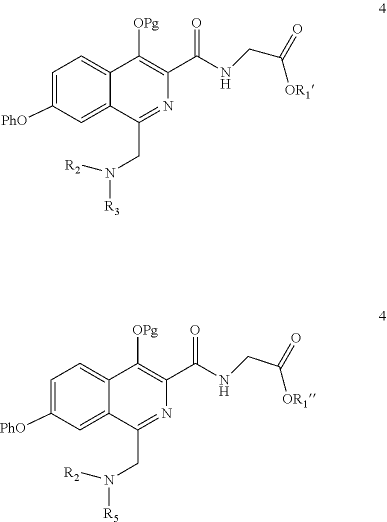

is prepared by:
(a) reacting a compound of formula 5′:

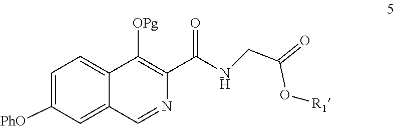

wherein Pg is H or an OH protecting group and R₁ is H, alkyl, aryl, or arylalkyl;
with an amine of formula:

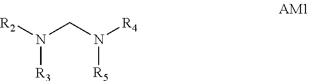

wherein:
R₂, R₃, R₄, and R₅ each independently represents alkyl, arylalkyl or alkenyl, or R₂ and R₃ and/or R₄ and R₅, taken together with the nitrogen atom to which they are bonded, each independently form a ring independently selected from:

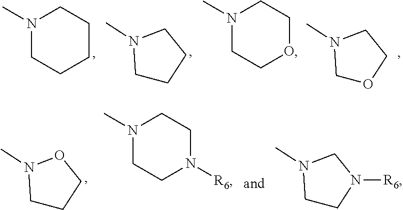

wherein R₆ is H or C₁₋₆ alkyl;
in the presence of an acid; and
(b) optionally, when the compound of formula 4′, formula 4″, or mixture thereof, has R₁═H, the compound or mixture is reacted with an alcohol R₁—OH, to form the compound of formula 4′, formula 4″ or mixture thereof, wherein R₁ is alkyl, aryl, or arylalkyl.
29. A process according to any of paragraphs 21, 22, 23, 24, 25, 26, 27, or 28, wherein Pg is H or an ester or ether OH protecting group.
30. A process according to paragraph 29, wherein Pg is H, methoxymethyl, tetrahydropyranyl, t-butyl, allyl, benzyl, benzyloxycarbonyl, t-butyldimethylsilyl, t-butyldiphenylsilyl; acetate, acetoxy, pivaloyl, or benzoyl.
31. A process according to paragraph 30, wherein Pg is H, benzyl, benzyloxycarbonyl, or acetoxy.
32. A process according to paragraph 31, wherein Pg is H.
33. A process according to any of paragraphs 21, 22, 23, 24, 25, 26, 27, 28, 29, 30, 31, or 32, wherein R₁ is H, C₁₋₆ alkyl, C₆₋₁₀ aryl, or C₇₋₁₂ arylalkyl.
34. A process according to paragraph 33, wherein R₁ is H or C₁₋₄ alkyl.
35. A process according to paragraph 34, wherein R₁ is H, methyl or ethyl, or R₁ is H or methyl.
36. A process according to any of paragraphs 21, 22, 23, 24, 25, 26, 27, 28, 29, 30, 31, 32, 33, 34, or 35, wherein R₂, R₃, R₄, and R₅ each independently represents an alkyl, arylalkyl or alkenyl group.
37. A process according to paragraph 36, wherein R₂, R₃, R₄, and R₅ each independently represents C₁₋₆ alkyl, C₇₋₁₂ arylalkyl or C₂₋₆ alkenyl.
38. A process according to paragraph 37, wherein R₂, R₃, R₄, and R₅ each independently represents C₁₋₆ alkyl; optionally C₁₋₃ alkyl; or
39. A process according to paragraph 38, wherein R₂, R₃, R₄, and R₅ are each methyl.
40. A process according to any of paragraphs 21, 22, 23, 24, 25, 26, 27, 28, 29, 30, 31, 32, 33, 34, 35, 36, 37, 38, or 39, wherein:
   R₂ and R₃ and/or R₄ and R₅ taken together with the nitrogen atom to which they are bonded, each independently form a ring selected from:

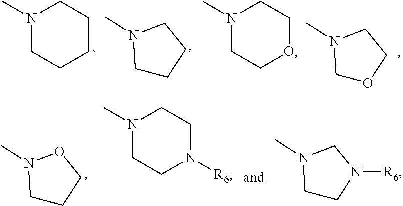

wherein R₆ is methyl; or
41. A process according to paragraph 40, wherein:R₂ and R₃, and/or R₄ and R₅, taken together with the nitrogen atom to which they are bonded, each independently form a ring selected from:

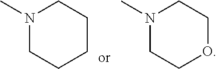

Optionally, the compound of formula 4′ is not present in a mixture with 4″.
42. Thus, also disclosed is a process according to any of paragraphs 1, 5, 6, 7, 8, 9, 10, 11, 12, 13, 14, 15, 16, 17, 18, 19, 20, 21, 22, 23, 24, 25, 26, 27, or 28, wherein the compound of formula 4′:

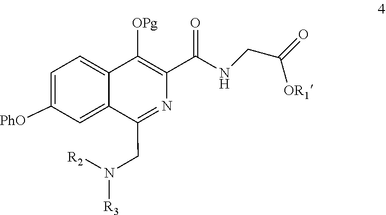

is prepared by:
(a) reacting a compound of formula 5′:

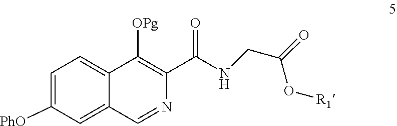

wherein Pg is H or an OH protecting group and R₁ is H, alkyl, aryl, or arylalkyl;
with an amine of formula:

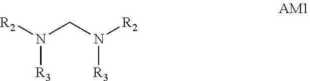

wherein:
R₂, and R₃, each independently represents alkyl, arylalkyl or alkenyl group, or R₂ and R₃ taken together with the nitrogen atom to which they are bonded, each independently form a ring independently selected from:

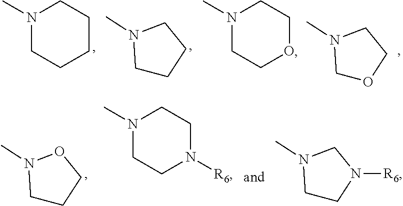

wherein R₆ is H or C₁₋₆ alkyl;
in the presence of an acid; and
(b) optionally, when the compound of formula 4′ has R₁═H, the compound or mixture is reacted with an alcohol R₁—OH, to form the compound of formula 4′ wherein R₁ is alkyl, aryl, or arylalkyl.
43. A process according to any of paragraphs 1, 5, 6, 7, 8, 9, 10, 11, 12, 13, 14, 15, 16, 17, 18, 19, 20, 21, 22, 23, 24, 25, 26, 27, or 28, wherein the compound of formula 4′:

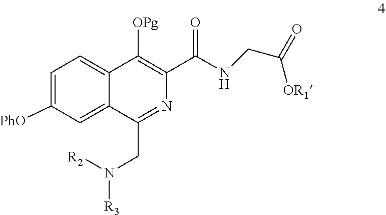

wherein Pg is H or an OH protecting group and R₁ is H, alkyl, aryl, or arylalkyl; R₂ is C₁₋₄ alkyl and R₃ is C₁₋₄ alkoxy; or R₂ and R₃ taken together with the nitrogen atom to which they are bonded, form a ring selected from:

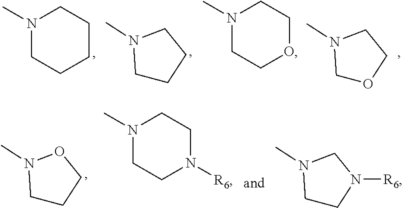

wherein R₆ is H or C₁₋₆ alkyl;
is prepared by a process comprising:
(a) reacting a compound of formula 5′:

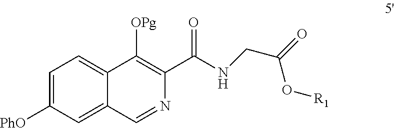

wherein Pg is H or an OH protecting group and R₁ is H, alkyl, aryl, or arylalkyl;
with formaldehyde or paraformaldehyde and an amine of formula:

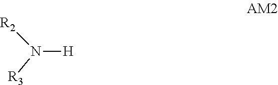

wherein R₂ is C₁₋₄ alkyl and R₃ is C₁₋₄ alkoxy; or R₂ and R₃ taken together with the nitrogen atom to which they are bonded, form a ring selected from:

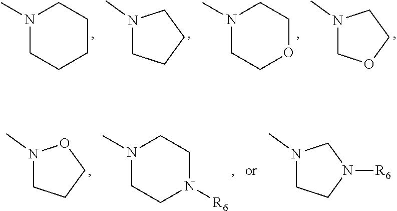

wherein R₆ is H or C₁₋₆ alkyl; in the presence of an acid, and
(b) optionally when R₁ is H, the compound of formula 4′ may be reacted with an alcohol R₁—OH, to form the compound of formula 4′ wherein R₁ is alkyl, aryl, or arylalkyl.
44. A process according to paragraph 43, wherein the reaction in step (a) is carried out using paraformaldehyde, in the presence of acetic acid or glacial acetic acid.
45. A process according to any of paragraphs 42, 43 or 44, wherein Pg is H or an ester or ether OH protecting group.
46. A process according to paragraph 45, wherein Pg is H or a OH protecting group selected from the group consisting of methoxymethyl, tetrahydropyranyl, t-butyl, allyl, benzyl, benzyloxycarbonyl, t-butyldimethylsilyl, t-butyldiphenylsilyl; acetate, acetoxy, pivaloyl, or benzoyl.
47. A process according to paragraph 46, wherein Pg is H, benzyl, benzyloxycarbonyl, or acetoxy.
48. A process according to paragraph 47, wherein Pg is H.
49. A process according to any of paragraphs 42, 43, 44, 45, 46, 47, or 48, wherein R₁ is H, C₁₋₆ alkyl, C₆₋₁₀ aryl, or C₇₋₁₂ arylalkyl.
50. A process according to paragraph 49, wherein R₁ is H or C₁₋₄ alkyl.
51. A process according to paragraph 46, wherein R₁ is H, methyl or ethyl, or wherein R₁ is H or methyl.
52. A process according to any of paragraphs 42, 43, 44, 45, 46, 47, 48, 49, 50, or 51, wherein R₂ and R₃ taken together with the nitrogen atom to which they are bonded, each independently form a ring selected from:

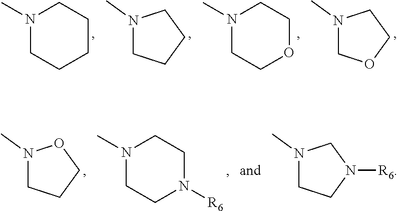

wherein R₆ is methyl.
53. A process according to paragraph 52, R₂ and R₃ taken together with the nitrogen atom to which they are bonded, each independently form a ring selected from:

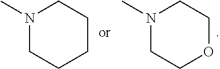

The compound of formula 5′ may be prepared by the following processes:
54. A process according to any of paragraphs 42, 43, 44, 45, 46, 47, 48, 49, 50, 51, 52, or 53, wherein the compound of formula 5′:

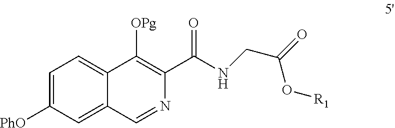

wherein Pg is H, and R₁ is H, alkyl, aryl, or arylalkyl;
is prepared by a process comprising:
(i) reacting a compound of formula 8:

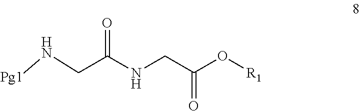

wherein Pg1 is a protecting group, optionally wherein Pg1 is —SO₂—Y,
wherein Y is alkyl, aryl or alkylaryl; and R₁ is H, alkyl, aryl, or arylalkyl;
with a compound of formula 9:

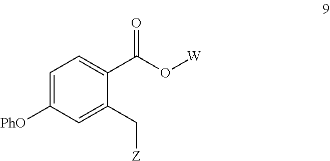

wherein W is alkyl, aryl or arylalkyl, and Z is a leaving group which is preferably halo, mesylate, tosylate, or besylate;
to form a compound of formula 10:

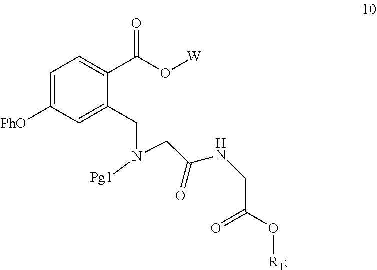

(ii) cyclising the compound of formula 10; and
(iii) optionally when R₁ is other than H, hydrolysing the compound of formula 5′ to form the compound of formula 5′ wherein R₁ is H.
55. A process according to paragraph 54, wherein Pg is H or an ester or ether OH protecting group.
56. A process according to paragraph 55, wherein Pg is H, methoxymethyl, tetrahydropyranyl, t-butyl, allyl, benzyl, benzyloxycarbonyl, t-butyldimethylsilyl, t-butyldiphenylsilyl; acetate, acetoxy, pivaloyl, or benzoyl.
57. A process according to paragraph 56, wherein Pg is H, benzyl, benzyloxycarbonyl, or acetoxy.
58. A process according to paragraph 57, wherein Pg is H.
59. A process according to any of paragraphs 54, 55, 56, 57, or 58, wherein R₁ is H, C₁₋₆ alkyl, C₆₋₁₀ aryl, or C₇₋₁₂ arylalkyl.
60. A process according to paragraph 59, wherein R₁ is H or C₁₋₄ alkyl.
61. A process according to paragraph 60, wherein R₁ is H or C₁₋₄ alkyl.
62. A process according to paragraph 61, wherein R₁ is H, methyl, or ethyl, or wherein R₁ is H or methyl.
63. A process according to any of paragraphs 54, 55, 56, 57, 58, 59, 60, 61, or 62, wherein Pg1 is —SO₂—Y, wherein Y is C₁₋₆ alkyl, C₆₋₁₀ aryl or C₇₋₁₂ alkylaryl.
64. A process according to paragraph 63, wherein Pg1 is —SO₂—Y, wherein Y is methyl or tolyl, or wherein Y is para-tolyl.
65. A process according to any of paragraphs 54, 55, 56, 57, 58, 59, 60, 61, 62, 63, or 64, wherein R₁ is C₁₋₆ alkyl, C₆₋₁₀ aryl, or C₇₋₁₂ arylalkyl;
66. A process according to paragraph 65, wherein R₁ is C₁₋₆ alkyl, or C₁₋₃ alkyl, or wherein R₁ is methyl.
67. A process according to paragraph 66, wherein Pg1 is tosyl or mesyl.
68. A process according to any of paragraphs 54, 55, 56, 57, 58, 59, 60, 61, or 62, 63, 64, 65, 66, or 67, wherein W is C₁₋₆ alkyl.
69. A process according to paragraph 68, wherein W is methyl.
70. A process according to any of paragraphs 54, 55, 56, 57, 58, 59, 60, 61, or 62, 63, 64, 65, 66, 67, 68, or 69, wherein Z is chloro, bromo or iodo; optionally wherein Z is chloro.
71. A process according to any of paragraphs 54, 55, 56, 57, 58, 59, 60, 61, or 62, 63, 64, 65, 66, 67, 68, 69, or 70, wherein W is methyl and Z is chloro.
72. A process according to any of paragraphs 54, 55, 56, 57, 58, 59, 60, 61, or 62, 63, 64, 65, 66, 67, 68, 69, 70, or 71, wherein in the compound of formula 8 is reacted with the compound of formula 9 in the presence of a base and a solvent.
73. A process according to paragraph 72, wherein the base is an alkaline metal carbonate or alkaline metal hydrogencarbonate.
74. A process according to paragraph 73, wherein the base is potassium carbonate, sodium carbonate, potassium hydrogen carbonate, or sodium hydrogen carbonate.
75. A process according to paragraph 74, wherein the base is potassium carbonate or sodium carbonate, or optionally wherein the base is potassium carbonate.
76. A process according to any of paragraphs 72, 73, 74, or 75, wherein the solvent is an aprotic solvent, optionally a dipolar aprotic solvent.
77. A process according to paragraph 76, wherein the solvent is N,N-dimethylformamide, DMSO, acetonitrile, or hexamethylphosphoramide, or optionally wherein the solvent is N,N-dimethylformamide, DMSO or acetonitrile, or wherein the solvent is N,N-dimethylformamide.
78. A process according to any of paragraphs 54, 55, 56, 57, 58, 59, 60, 61, or 62, 63, 64, 65, 66, 67, 68, 69, 70, 71, 72, 73, 74, 75, 76, or 77, wherein the cyclisation is carried out using an alkaline metal alkoxide in the presence of a solvent.
79. A process according to paragraph 78, wherein the alkaline metal alkoxide is selected from sodium methoxide, potassium methoxide, sodium ethoxide or potassium ethoxide, or optionally wherein the alkaline metal alkoxide is selected from sodium methoxide or potassium methoxide, or optionally wherein the alkaline metal alkoxide is sodium methoxide.
80. A process according to paragraph 78 or 79, wherein the solvent is an alcohol or a dipolar aprotic solvent; optionally a C₁₋₄ alcohol, N,N-dimethylformamide, DMSO or acetonitrile; or optionally wherein the solvent is a methanol or N,N-dimethylformamide.
81. A process according to any of paragraphs 54, 55, 56, 57, 58, 59, 60, 61, or 62, 63, 64, 65, 66, 67, 68, 69, 70, 71, 72, 73, 74, 75, 76, 77, 78, 79, or 80, wherein step (ii) is carried out without isolation of the compound 10 from the reaction mixture in step (i).
82. A process according to any of paragraphs 54, 55, 56, 57, 58, 59, 60, 61, or 62, 63, 64, 65, 66, 67, 68, 69, 70, 71, 72, 73, 74, 75, 76, 77, 78, 79, 80, or 81, wherein step (iii) is carried out to prepare a compound of formula 5 wherein R₁ is H.
83. A process according to any of paragraphs 42, 42, 44, 45, 46, 47, 48, 49, 50, 51, 52, or 53, wherein the compound of formula 5′:

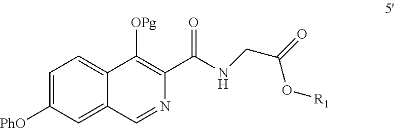

wherein Pg is H or a OH protecting group, and R₁ is H, alkyl, aryl, arylalkyl; is prepared by a process comprising:
(i) reacting a compound of formula 7′:

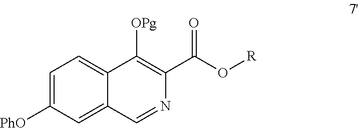

wherein Pg is H or an OH protecting group and R is H, alkyl, aryl, arylalkyl;
with the compound:

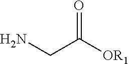

wherein R₁ is H, alkyl, aryl, or arylalkyl;
to form the compound of formula 5′: and
(ii) optionally, when the compound of formula 5′ has R₁═H, the compound may be reacted with an alcohol R₁—OH, to form the compound of formula 5′ wherein R₁ is alkyl, aryl, or arylalkyl.
84. A process according to paragraph 83, wherein Pg is H or an ester or ether OH protecting group.
85. A process according to paragraph 84, wherein Pg is is H or a OH protecting group selected from the group consisting of methoxymethyl, tetrahydropyranyl, t-butyl, allyl, benzyl, benzyloxycarbonyl, t-butyldimethylsilyl, t-butyldiphenylsilyl; acetate, acetoxy, pivaloyl, or benzoyl.
86. A process according to paragraph 85, wherein Pg is H, benzyl, benzyloxycarbonyl, or acetoxy.
87. A process according to paragraph 86, wherein Pg is H.
88. A process according to any of paragraphs 83, 84, 85, 86, or 87, wherein R₁ is H, C₁₋₆ alkyl, C₆₋₁₀ aryl, or C₇₋₁₂ arylalkyl.
89. A process according to paragraph 88, wherein R₁ is H or C₁₋₄ alkyl.
90. A process according to paragraph 89, wherein R₁ is H, methyl or ethyl, or wherein R₁ is H or methyl.
91. A process according to any of paragraphs 83, 84, 85, 86, 87, 88, 89, or 90, wherein: R is H or C₁₋₆ alkyl.
92. A process according to paragraph 91, wherein R is H or C₁₋₃ alkyl.
93. A process according to paragraph 92 wherein R is H, methyl or ethyl, or wherein R₁ is H or methyl.
   The disclosure encompasses a process for preparing Roxadustat using compounds 8 and 9 as starting materials as described above. This process is described below:
94. A process for the preparation of Roxadustat or a salt thereof, comprising:
   (a) reacting a compound of formula 8:

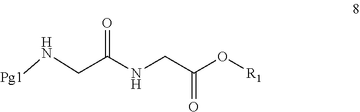

wherein Pg1 is a protecting group, optionally wherein Pg1 is —SO₂—Y,
wherein Y is alkyl, aryl or alkylaryl; and R₁ is H, alkyl, aryl, or arylalkyl;
with a compound of formula 9:

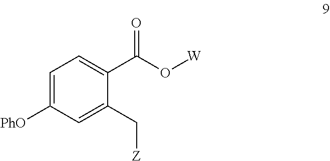

wherein W is alkyl, aryl or arylalkyl, and Z is a leaving group which is preferably halo, mesylate, tosylate, or besylate;
to form a compound of formula 10:

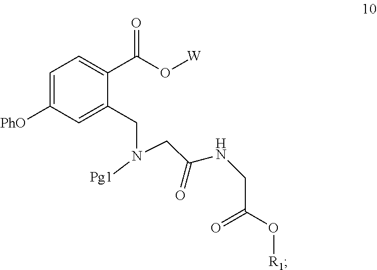

and
(b) cyclising the compound of formula 10 to form the compound of formula 5′

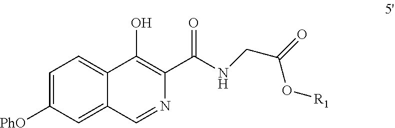

wherein R₁ is H, alkyl, aryl, arylalkyl;
(c) optionally when R₁ is other than H, hydrolysing the compound of formula 5′ to form the compound of formula 5′ wherein R₁ is H.
(d) reacting the compound of formula 5′ with formaldehyde or paraformaldehyde and an amine of formula:

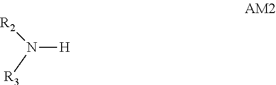

wherein R₂ is C₁₋₄ alkyl and R₃ is C₁₋₄ alkoxy; or R₂ and R₃ taken together with the nitrogen atom to which they are bonded, form a ring selected from:

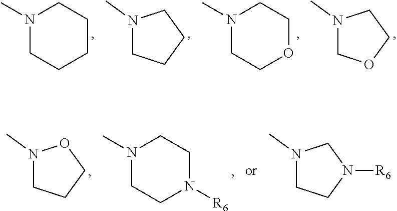

wherein R₆ is H or C₁₋₆ alkyl; in the presence of an acid; to form a compound of formula 4′:

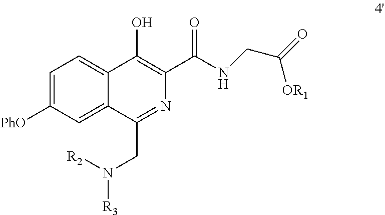

wherein R₁ is H, alkyl, aryl, or arylalkyl; R₂ is C₁₋₄ alkyl and R₃ is C₁₋₄ alkoxy; or R₂ and R₃ taken together with the nitrogen atom to which they are bonded form a ring selected from:

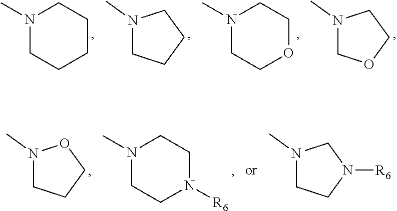

wherein R₆ is H or C₁₋₆ alkyl;
(e) optionally wherein when compound of 4′ has R₁═H, the compound is reacted with an alcohol R₁—OH, to form the compound of formula 4′ wherein R₁ is alkyl, aryl, or arylalkyl;
(f) reducing the compound of formula 4′ to form a compound of formula 2′:

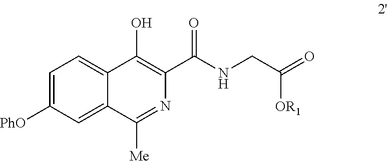

wherein R₁ is H, alkyl, aryl, or arylalkyl; and
(g) optionally wherein when the compound of 2′ has R₁=alkyl, aryl, or arylalkyl, hydrolysing the compound of formula 2′ to form Roxadustat (1):

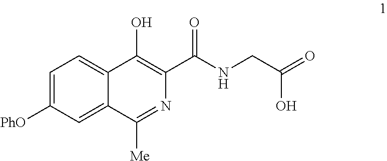 [SMILES: Cc1nc(C(=O)NCC(=O)O)c(O)c2ccc(Oc3ccccc3)cc12]

optionally wherein the hydrolysis is carried out using a base and one or more polar solvents; and
(h) optionally converting Roxadustat to a salt.
95. A process according to paragraph 94, wherein Pg1 is —SO₂—Y, wherein Y is C₁₋₆ alkyl, C₆₋₁₀ aryl or C₇₋₁₂ alkylaryl.
96. A process according to paragraph 95, wherein Pg1 is —SO₂—Y, wherein Y is methyl or tolyl, or wherein Y is para-tolyl.
97. A process according to any of paragraphs 94, 95, or 96, wherein R₁ is C₁₋₆ alkyl, C₆₋₁₀ aryl, or C₇₋₁₂ arylalkyl.
98. A process according to paragraph 97, wherein R₁ is C₁₋₆ alkyl, or C₁₋₃ alkyl, or wherein R₁ is methyl.
99. A process according to any of paragraphs 94, 95, 96, 97, or 98, wherein W is C₁₋₆ alkyl, optionally wherein W is methyl.
100. A process according to any of paragraphs 94, 95, 96, 97, 98, or 99, wherein Z is chloro, bromo or iodo; optionally wherein Z is chloro.
101. A process according to any of paragraphs 94, 95, 96, 97, 98, 99, or 100, wherein R₂ is C₁₋₄ alkyl and R₃ is C₁₋₄ alkoxy; or wherein R₂ is methyl and R₃ is methoxy.
102. A process according to any of paragraphs 94, 95, 96, 97, 98, 99, 100, or 101, wherein R₂ and R₃ taken together with the nitrogen atom to which they are bonded, form a ring selected from:

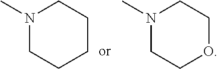

103. A process according to paragraph 102, wherein R₂ and R₃ taken together with the nitrogen atom to which they are bonded, form:

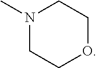

104. A process according to any of paragraphs 94, 95, 96, 97, 98, 99, 100, 101, 102, or 103, wherein step (a) is carried out in the presence of a base and a solvent.
105. A process according to paragraph 104, wherein the base is an alkaline metal carbonate or alkaline metal hydrogencarbonate.
106. A process according to paragraph 105, wherein the base is selected form potassium carbonate, sodium carbonate, potassium hydrogen carbonate, or optionally potassium carbonate or sodium carbonate, or sodium hydrogen carbonate.
107. A process according to any of paragraphs 104, 105 or 106, wherein the solvent is an aprotic solvent, optionally a dipolar aprotic solvent, and optionally N,N-dimethylformamide, DMSO, acetonitrile, or hexamethylphosphoramide
108. A process according to paragraph 107, wherein the solvent is N,N-dimethylformamide, DMSO or acetonitrile; or wherein the solvent is N,N-dimethylformamide.
109. A process according to any of paragraphs 94, 95, 96, 97, 98, 99, 100, 101, 102, 103, 104, 105, 106, 107, or 108, wherein step (b) is carried out using an alkaline metal alkoxide in the presence of a solvent.
110. A process according to paragraph 109, wherein the alkaline metal alkoxide is selected from sodium methoxide, potassium methoxide, sodium ethoxide or potassium ethoxide; optionally wherein the alkaline metal alkoxide is sodium methoxide or potassium methoxide; or wherein the alkaline metal alkoxide is sodium methoxide.
111. A process according to paragraph 109 or paragraph 110, wherein the solvent is an alcohol or a dipolar aprotic solvent, optionally wherein the solvent is a C₁₋₄ alcohol, N,N-dimethylformamide, DMSO or acetonitrile; or wherein the solvent is methanol or N,N-dimethylformamide.
112. A process according to any of paragraphs 94, 95, 96, 97, 98, 99, 100, 101, 102, 103, 104, 105, 106, 107, 108, 109, 110, or 111, wherein step (b) is carried out without isolation of the compound 10 from the reaction mixture in step (a).
113. A process according to any of paragraphs 94, 95, 96, 97, 98, 99, 100, 101, 102, 103, 104, 105, 106, 107, 108, 109, 110, 111, or 112, wherein step (c) is carried out to prepare a compound of formula 5′ wherein R₁ is H.
114. A process according to any of paragraphs 94, 95, 96, 97, 98, 99, 100, 101, 102, 103, 104, 105, 106, 107, 108, 109, 110, 111, 112, or 113, wherein step (d) is carried out using paraformaldehyde, optionally wherein the acid is acetic acid, or glacial acetic acid.
115. A process according to any of paragraphs 94, 95, 96, 97, 98, 99, 100, 101, 102, 103, 104, 105, 106, 107, 108, 109, 110, 111, 112, 113, or 114, wherein R₁ in compound 4′ is H and wherein step (e) is carried out.
116. A process according to any of paragraphs 94, 95, 96, 97, 98, 99, 100, 101, 102, 103, 104, 105, 106, 107, 108, 109, 110, 111, 112, 113, or 114, wherein step (f) is carried out using zinc and an acid, optionally zinc and hydrochloric acid, or zinc and concentrated hydrochloric acid.
117. A process according to any of paragraphs 94, 95, 96, 97, 98, 99, 100, 101, 102, 103, 104, 105, 106, 107, 108, 109, 110, 111, 112, 113, 114, 115, or 116, wherein step (f) is carried out using zinc and an acid, optionally hydrogen gas and Pd/catalyst.
118. A process according to any of paragraphs 94, 95, 96, 97, 98, 99, 100, 101, 102, 103, 104, 105, 106, 107, 108, 109, 110, 111, 112, 113, 114, 115, 116, or 117, wherein step (f) is carried out in a polar solvent, wherein the polar solvent is optionally a chlorinated hydrocarbon, a ketone or an ester, and optionally a chlorinated C₁₋₃ alkane, or wherein the polar solvent is dichloromethane.
119. A process according to any of paragraphs 94, 95, 96, 97, 98, 99, 100, 101, 102, 103, 104, 105, 106, 107, 108, 109, 110, 111, 112, 113, 114, 115, 116, 117, or 118, wherein step (g) is carried out in the presence of a base and one or more polar solvents.
120. A process according to paragraph 119, wherein the base is an alkaline metal hydroxide, optionally sodium hydroxide or potassium hydroxide, and optionally sodium hydroxide.
121. A process according to paragraph 119 or 120, wherein the one or more polar solvents is selected from the group consisting of an ether, a ketone, an alcohol, or water, optionally wherein the solvent comprises a cyclic ether, an aliphatic alcohol and water, or wherein the solvent comprises THF, ethanol and water.
121. A process according to any of paragraphs 1 to 121, further comprising combining the Roxadustat or salt thereof with at least one pharmaceutically acceptable excipient to form a pharmaceutical composition.
   Also disclosed is the use of the various compounds as described above in a process for preparing Roxadustat. Accordingly the present disclosure includes:
122. Use of:
   (i) a compound of formula 3′:

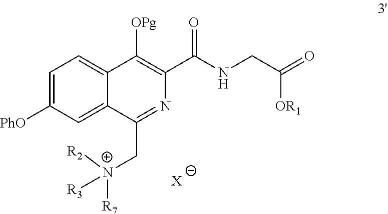

wherein:
Pg is H or an OH protecting group;
R₁ is C₁₋₆ alkyl, C₆₋₁₀ aryl, or C₇₋₁₂ arylalkyl;
R₂ and R₃, each independently represents C₁₋₆ alkyl, C₇₋₁₂ arylalkyl or C₂₋₆ alkenyl; or
R₂ and R₃ taken together with the nitrogen atom to which they are bonded, each independently form a ring selected from:

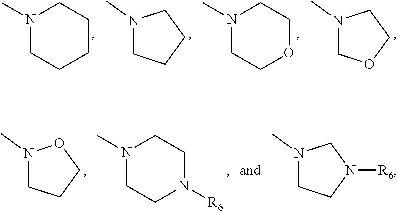

wherein R₆ is H or C₁₋₆ alkyl;
R₇ is C₁ to C₆ alkyl; and
and X⁻ is an anion selected from the group consisting of halide, O—SO₄—R₇ wherein R₇ is C₁ to C₆ alkyl, or O—SO₂—R₈ wherein R₈ is phenyl, tolyl, methyl or trifluoromethyl; or
(ii) a compound of formula 4′:

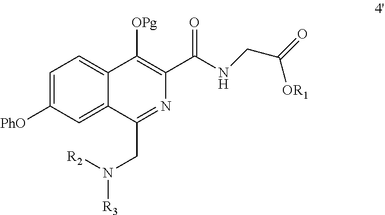

wherein:
Pg is H or an OH protecting group;
R₁ is H, C₁₋₆ alkyl, C₆₋₁₀ aryl, or C₇₋₁₂ arylalkyl; R₂ and R₃ each independently represents C₁₋₆ alkyl, C₆₋₁₀ aryl, C₇₋₁₂ arylalkyl or C₂₋₆ alkenyl; or R₂ is C₁₋₄ alkyl and R₃ is C₁₋₄ alkoxy; or R₂ and R₃ taken together with the nitrogen atom to which they are bonded, each independently form a ring selected from:

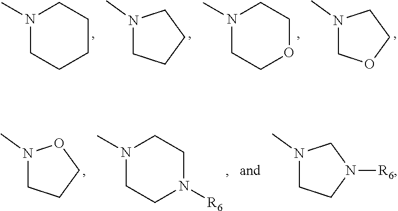

wherein R₆ is H or C₁₋₆ alkyl; or
(iii) a compound of formula 5:

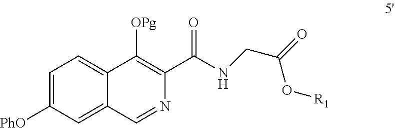

wherein:
Pg is H, or an OH protecting group; and
R₁ is H, C₁₋₆ alkyl, C₆₋₁₀ aryl or C₇₋₁₂ arylalkyl; or
(iv) a compound of formula 8:

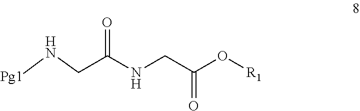

wherein:
Pg1 is a protecting group, optionally wherein Pg1 is —SO₂—Y, wherein Y is C₁₋₆ alkyl, C₆₋₁₀ aryl or C₇₋₁₂ alkylaryl; and R₁ is H, C₁₋₆ alkyl, C₆₋₁₀ aryl, or C₇₋₁₂ arylalkyl; and
R₁ is H, C₁₋₆ alkyl, C₆₋₁₀ aryl or C₇₋₁₂ arylalkyl; or
(v) a compound of formula 9:

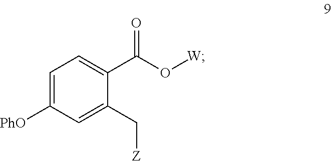

wherein:
W is C₁₋₆ alkyl, C₆₋₁₀ aryl or C₇₋₁₂ arylalkyl, and
Z is a leaving group which is preferably halo, mesylate, tosylate, or besylate; or
(vi) a compound of formula 10:

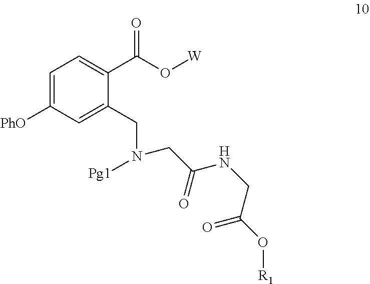

wherein:
Pg1 is a protecting group, optionally wherein Pg1 is —SO₂—Y, wherein Y is C₁₋₆ alkyl, C₆₋₁₀ aryl or C₇₋₁₂ alkylaryl; and R₁ is H, C₁₋₆ alkyl, C₆₋₁₀ aryl, or arylalkyl;
R₁ is H, C₁₋₆ alkyl, C₆₋₁₀ aryl or C₇₋₁₂ arylalkyl; and
W is C₁₋₆ alkyl, C₆₋₁₀ aryl or C₇₋₁₂ arylalkyl;
in a process for the preparation of Roxadustat or a salt thereof.
123. Use according to paragraph 122 of:
   (i) a compound of formula 3′:

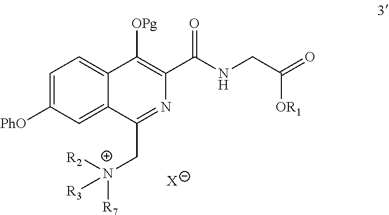

wherein:
Pg is H, methoxymethyl, tetrahydropyranyl, t-butyl, allyl, benzyl, benzyloxycarbonyl, t-butyldimethylsilyl, t-butyldiphenylsilyl, acetate, acetoxy, pivaloyl, or benzoyl; or Pg is selected from H, benzyl, benzyloxycarbonyl or acetoxy;
R₁ is C₁₋₃ alkyl;
R₂ and R₃ each independently represents C₁₋₃ alkyl; or
R₂ and R₃ taken together with the nitrogen atom to which they are bonded, each independently form a ring selected from:

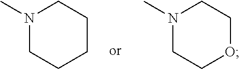

R₇ is C₁ to C₃ alkyl; and
X⁻ is an anion selected from the group consisting of halide or O—SO₄—R₇ wherein R₇ is C₁ to C₃ alkyl; or
(ii) a compound of formula 4′:

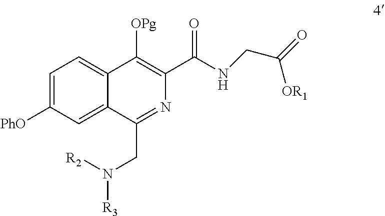

wherein Pg is H, methoxymethyl, tetrahydropyranyl, t-butyl, allyl, benzyl, benzyloxycarbonyl, t-butyldimethylsilyl, t-butyldiphenylsilyl, acetate, acetoxy, pivaloyl, or benzoyl; or Pg is selected from H, benzyl, benzyloxycarbonyl or acetoxy;
R₁ is H or C₁₋₃ alkyl; and
R₂ and R₃, each independently represents C₁₋₃ alkyl; or R₂ is C₁₋₄ alkyl and
R₃ is C₁₋₄ alkoxy; R₂ is C₁₋₄ alkyl and R₃ is C₁₋₄ alkoxy; or R₂ and R₃, taken together with the nitrogen atom to which they are bonded, each independently form a ring selected from:

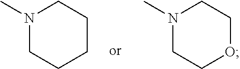

or
(iii) a compound of formula 5:

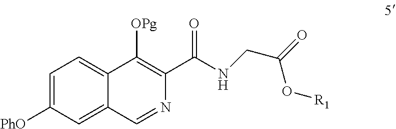

wherein:
Pg is H, methoxymethyl, tetrahydropyranyl, t-butyl, allyl, benzyl, benzyloxycarbonyl, t-butyldimethylsilyl, t-butyldiphenylsilyl, acetate, acetoxy, pivaloyl, or benzoyl; or Pg is selected from H, benzyl, benzyloxycarbonyl or acetoxy; and
R₁ is H or C₁₋₃ alkyl; or
(iv) a compound of formula 8:

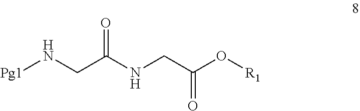

wherein:
Pg1 is —SO₂—Y, wherein Y is C₁₋₃ alkyl, C₆₋₈ aryl or C₇₋₉ alkylaryl; and R₁ is H, C₁₋₃ alkyl, C₆₋₈ aryl, or C₇₋₉ arylalkyl; or
(v) a compound of formula 9:

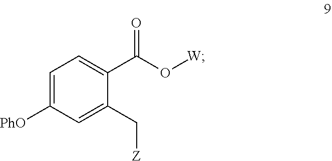

wherein:
W is C₁₋₃ alkyl; and
Z is chloro, bromo or iodo, optionally wherein Z is chloro; or
(vi) a compound of formula 10:

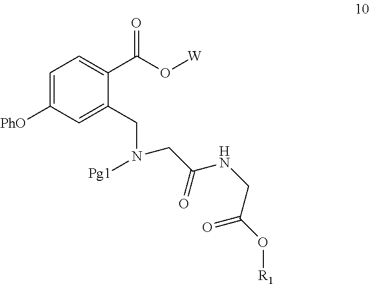

wherein:
Pg1 is SO₂—Y, wherein Y is C₁₋₃ alkyl, C₆₋₈ aryl or C₇₋₉ alkylaryl; and R₁ is H, C₁₋₃ alkyl, C₆₋₈ aryl, or C₇₋₉ arylalkyl;
W is C₁₋₃ alkyl, C₆₋₈ aryl or C₇₋₉ arylalkyl;
in a process for the preparation of Roxadustat or a salt thereof.
124. The use according to paragraph 123 of:
   (i) a compound of formula 3′:

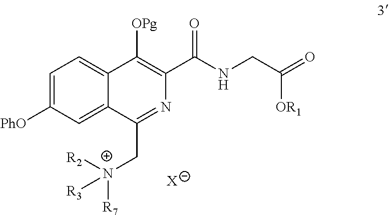

wherein:
Pg is H, benzyl, benzyloxycarbonyl, or acetoxy;
R₁ is methyl;
R₂ and R₃ are each methyl; or
R₂ and R₃ taken together with the nitrogen atom to which they are bonded, each independently form a ring selected from:

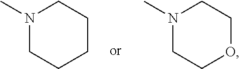

and
R₇ is methyl; and
and X⁻ is an anion selected from the group consisting of chloro, bromo or iodo, or O—SO₄—R₇ wherein R₇ is C₁ to C₃ alkyl; or
(ii) a compound of formula 4′:

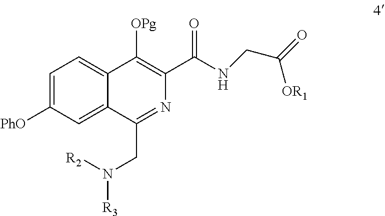

wherein Pg is H, benzyl, benzyloxycarbonyl, or acetoxy;
R₁ is H, methyl or ethyl; or
R₂ is methyl and R₃ is methoxy; or
R₂ and R₃, each independently represents methyl; or R₂ and R₃, taken together with the nitrogen atom to which they are bonded, each independently form a ring selected from:

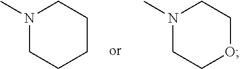

or
(iii) a compound of formula 5:

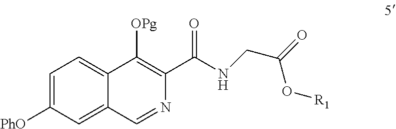

wherein:
Pg is H, benzyl, benzyloxycarbonyl, or acetoxy; and
R₁ is H, methyl or ethyl; or
(iv) a compound of formula 8:

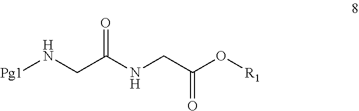

wherein:
Pg1 is —SO₂—Y, wherein Y is methyl or tolyl; and
R₁ is H, methyl or ethyl; or
(v) a compound of formula 9:

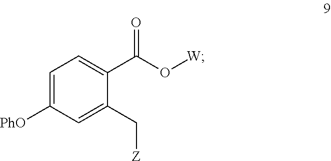

or
   wherein:
   W is methyl; and Z is chloro; or
(vi) a compound of formula 10:

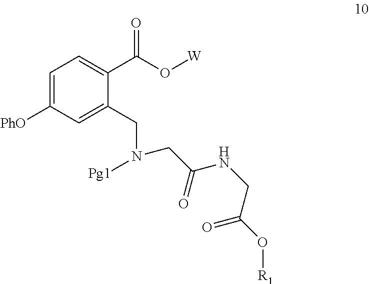

wherein:
Pg1 is SO₂—Y, wherein Y is methyl or tolyl;
R₁ is H, methyl or ethyl; and
W is methyl;
in a process for the preparation of Roxadustat or a salt thereof.
   Also disclosed are processes for preparing the compounds which are useful intermediates in the disclosed processes for preparing Roxadustat, as described below:
125. A process for preparing a compound of formula 5′:

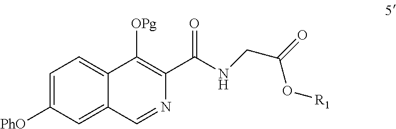

wherein: Pg is H or an OH protecting group and R₁ is H, alkyl, aryl, arylalkyl; comprising:

Option A
(a) reacting a compound of formula 8:

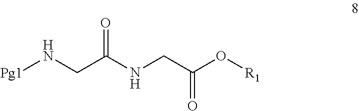

wherein Pg1 is a protecting group, optionally wherein Pg1 is —SO₂—Y,
wherein Y is alkyl, aryl or alkylaryl; and R₁ is H, alkyl, aryl, or arylalkyl;
with a compound of formula 9:

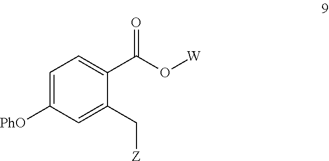

wherein W is alkyl, aryl or arylalkyl, and Z is a leaving group which is preferably halo, mesylate, tosylate, or besylate;
to form a compound of formula 10:

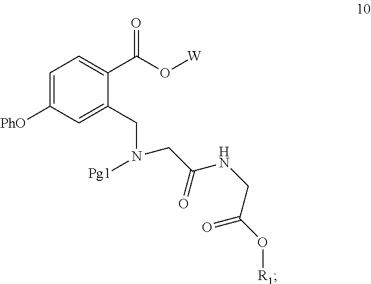

and
(b) cyclising the compound of formula 10 to form the compound of formula 5′; and
(c) optionally when R₁ is other than H, hydrolysing the compound of formula 5′ to form the compound of formula 5′ wherein R₁ is H; or

Option B
(a) reacting a compound of formula 7′:

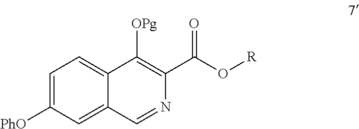

wherein Pg is H or an OH protecting group and R is H, alkyl, aryl, arylalkyl;
with the compound:

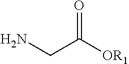

wherein R₁ is H, alkyl, aryl, or arylalkyl;
to form the compound of formula 5′: and
(b) optionally, when the compound of formula 5′ has R₁═H, the compound may be reacted with an alcohol R₁—OH, to form the compound of formula 5′ wherein R₁ is alkyl, aryl, or arylalkyl.
126. A process according to paragraph 125 for preparing a compound of formula 5′, comprising:
   (a) reacting a compound of formula 8:

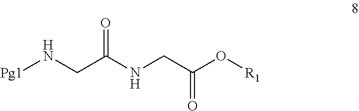

wherein Pg1 is a protecting group, optionally wherein Pg1 is —SO₂—Y,
wherein Y is alkyl, aryl or alkylaryl; and R₁ is H, alkyl, aryl, or arylalkyl; with a compound of formula 9:

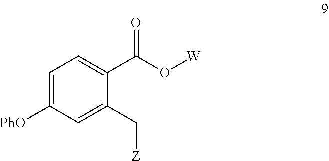

wherein W is alkyl, aryl or arylalkyl, and Z is a leaving group, preferably halo, mesylate, tosylate or besylate;
to form a compound of formula 10:

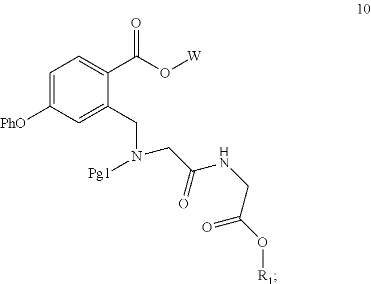

(b) cyclising the compound of formula 10 to form the compound of formula 5′; and
(c) optionally when R₁ is other than H, hydrolysing the compound of formula 5′ to form the compound of formula 5′ wherein R₁ is H.
127. A process according to paragraph 126, wherein:
   Pg1 is —SO₂—Y, wherein Y is C₁₋₆ alkyl, C₆₋₁₀ aryl or C₇₋₁₂ alkylaryl; and/or R₁ is C₁₋₆ alkyl, C₆₋₁₀ aryl, or C₇₋₁₂ arylalkyl;
   Pg1 is —SO₂—Y, wherein Y is methyl or tolyl, or wherein Y is para-tolyl; and/or
   R₁ is C₁₋₆ alkyl, or C₁₋₃ alkyl, or wherein R₁ is methyl; or
   Pg1 is tosyl and Y is mesyl.
128. A process according to paragraph 126 or paragraph 127, wherein:
   W is C₁₋₆ alkyl, optionally wherein W is C₁₋₃ alkyl; and Z is chloro, bromo, iodo; optionally wherein Z is chloro; or
   W is methyl and Z is chloro.
129. A process according to any of paragraphs 126, 127, or 128, wherein the compound of formula 8 is reacted with the compound of formula 9 in the presence of a base and a solvent.
130. A process according to paragraph 129, wherein:
   the base is an alkaline metal carbonate or alkaline metal hydrogencarbonate, optionally potassium carbonate, sodium carbonate, potassium hydrogen carbonate, or sodium hydrogen carbonate and/or the solvent is an aprotic solvent, optionally a dipolar aprotic solvent, optionally N,N-dimethylformamide, DMSO, acetonitrile, or hexamethylphosphoramide; or
   the base is potassium carbonate or sodium carbonate, and/or the solvent is N,N-dimethylformamide, DMSO or acetonitrile; or
   the base is potassium carbonate and/or the solvent is N,N-dimethylformamide.
131. A process according to any of paragraphs 126, 127, 128, 129, or 130, wherein the cyclisation is carried out using an alkaline metal alkoxide in the presence of a solvent.
132. A process according to paragraph 131, wherein:
   the alkaline metal alkoxide is selected from sodium methoxide, potassium methoxide, sodium ethoxide or potassium ethoxide and/or the solvent is an alcohol or a dipolar aprotic solvent;
   the alkaline metal alkoxide is selected from sodium methoxide or potassium methoxide and/or
   the solvent is a C₁₋₄ alcohol, N,N-dimethylformamide, DMSO or acetonitrile; or
   the alkaline metal alkoxide is sodium methoxide; and the solvent is a methanol or N,N-dimethylformamide.
133. A process according to any of paragraphs 126, 127, 128, 129, or 130, 131, or 132, wherein step (b) is carried out without isolation of the compound 10 from the reaction mixture in step (a).
134. A process according to any of paragraphs 126, 127, 128, 129, or 130, 131, 132, or 133, wherein step (c) is carried out to prepare a compound of formula 5′ wherein R₁ is H.
135. A process according to paragraph 125 for preparing a compound of formula 5′, comprising:
   (a) reacting a compound of formula 7′:

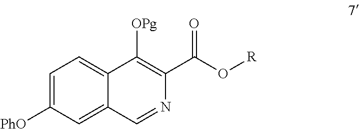

wherein Pg is H or an OH protecting group and R is H, alkyl, aryl, or arylalkyl;
with the compound:

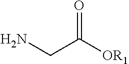

wherein R₁ is H, alkyl, aryl, or arylalkyl;
to form the compound of formula 5′; and
(b) optionally, when the compound of formula 5′ has R₁═H, the compound may be reacted with an alcohol R₁—OH, to form the compound of formula 5′ wherein R₁ is alkyl, aryl, or arylalkyl.
   The above reaction may be carried out in the presence of propanephosphonic acid anhydride, which may advantageously enable the coupling reaction to take place faster and/or at lower reaction temperatures.
136. A process according to paragraph 135, wherein:
   Pg is H or an ester or ether OH protecting group; and/or R₁ is H, C₁₋₆ alkyl, C₆₋₁₀ aryl, or C₇₋₁₂ arylalkyl; or optionally wherein R is H or C₁₋₃ alkyl;
   Pg is H, methoxymethyl, tetrahydropyranyl, t-butyl, allyl, benzyl, benzyloxycarbonyl, t-butyldimethylsilyl, t-butyldiphenylsilyl, acetate, acetoxy, pivaloyl, or benzoyl; and/or R₁ is H or C₁₋₆ alkyl, C₆₋₁₀ aryl, or C₇₋₁₂ arylalkyl;
   Pg is H, benzyl, benzyloxycarbonyl, or acetoxy; and/or
   R₁ is H or C₁₋₄ alkyl; or
   Pg is H and R₁ is H, methyl or ethyl;
137. A process according to paragraph 135 or paragraph 136, wherein the reaction is carried out in the presence of a base, optionally wherein the base is an organic amine, or an alkaline metal alkoxide, or wherein the base is selected triethylamine or sodium methoxide.
138. A process according to any of paragraphs 135, 136 or 137, wherein the reaction is carried out in a solvent, optionally wherein the solvent is selected from an alcohol or a chlorinated hydrocarbon, or an alcohol, or a chlorinated C₁-C₄ alkane, or wherein the reaction is carried out in methanol or dichloromethane.
139. A process for preparing a compound of formula 4′, a compound of formula 4″ or a mixture of 4′ and 4″:

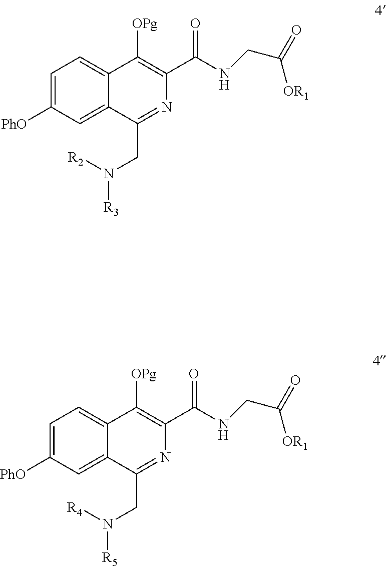

wherein Pg is H or an OH protecting group and R₁ is H, alkyl, aryl, or arylalkyl; R₂, R₃, R₄ and R₅ each independently represents alkyl, arylalkyl or alkenyl, R₂ and R₃ and/or R₄ and R₅, taken together with the nitrogen atom to which they are bonded, each independently form a ring selected from:

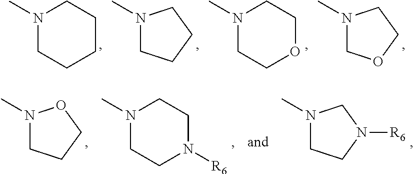

wherein R₆ is H or C₁₋₆ alkyl;
comprising:
(a) reacting a compound of formula 5′:

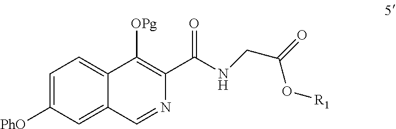

wherein Pg is H or an OH protecting group and R₁ is H, alkyl, aryl, or arylalkyl;
with an amine of formula:

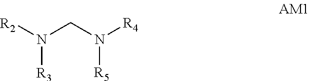

wherein:
R₂, R₃, R₄, and R₅ each independently represents alkyl, arylalkyl or alkenyl group, or R₂ and R₃ and/or R₄ and R₅, taken together with the nitrogen atom to which they are bonded, each independently form a ring independently selected from:

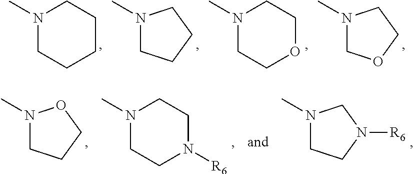

wherein R₆ is H or C₁₋₆ alkyl;
in the presence of an acid; and
(b) optionally, when the compound of formula 4′, formula 4″, or mixture thereof, has R₁═H, the compound or mixture is reacted with an alcohol R₁—OH, to form the compound of formula 4′, formula 4″ or mixture thereof, wherein R₁ is alkyl, aryl, or arylalkyl.
140. A process according to paragraph 139, wherein the amine AM1 is a symmetrical amine having the formula AM1′:

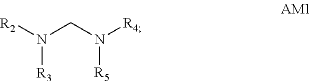

and wherein the product has the formula 4′:

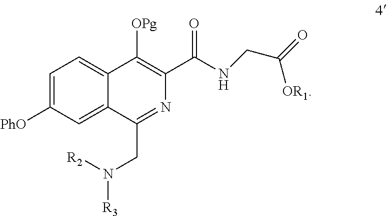

141. A process according to paragraph 139 or paragraph 140, wherein:
   Pg is H, or an ester or ether OH protecting group; optionally wherein Pg is H, methoxymethyl, tetrahydropyranyl, t-butyl, allyl, benzyl, benzyloxycarbonyl, t-butyldimethylsilyl, t-butyldiphenylsilyl, acetate, acetoxy, pivaloyl, or benzoyl; and/or R₁ is H, C₁₋₆ alkyl, C₆₋₁₀ aryl, or C₇₋₁₂ arylalkyl,
   Pg is H, benzyl, benzyloxycarbonyl, or acetoxy; and/or
   R₁ is C₁₋₃ alkyl; or
   Pg is H and R₁ is methyl or ethyl.
142. A process according to paragraph 139 or paragraph 141, wherein:
   R₂, R₃, R₄, and R₅ each independently represents C₁₋₆ alkyl, C₇₋₁₂ arylalkyl or C₂₋₆ alkenyl group; or
   R₂, R₃, R₄, and R₅ each independently represents C₁₋₆ alkyl; optionally C₁₋₃ alkyl, or
   R₂, R₃, R₄, and R₅ are each methyl.
143. A process according to paragraph 140, wherein:
   R₂ and R₃ each independently represents C₁₋₆ alkyl, C₇₋₁₂ arylalkyl or C₂₋₆ alkenyl group; or
   R₂ and R₃ each independently represents C₁₋₆ alkyl; optionally C₁₋₃ alkyl, or
   R₂ and R₃ are each methyl.
144. A process according to any of paragraphs 140, 141, 142, or 143, wherein:
   Pg is H or benzyl, and optionally H; and
   R₁ is H, methyl or ethyl; and
   R₂ and R₃ are each methyl.
      The compound of formula 4′ may optionally be prepared by a Mannich reaction as discussed below:
145. A process for preparing a compound of formula 4′:

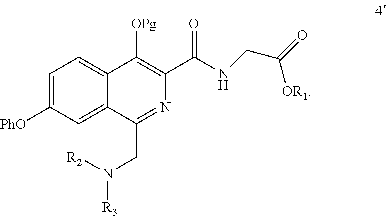

wherein Pg is H or an OH protecting group and R₁ is H, alkyl, aryl, or arylalkyl;
R₂ is C₁₋₄ alkyl and R₃ is C₁₋₄ alkoxy; or R₂ and R₃ taken together with the nitrogen atom to which they are bonded, form a ring selected from:

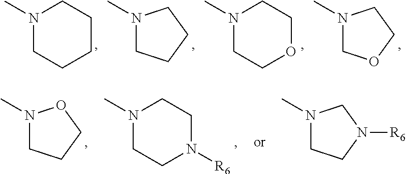

wherein R₆ is H or C₁₋₆ alkyl;
comprising:
(a) reacting a compound of formula 5′:

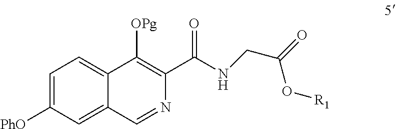

wherein Pg is H or an OH protecting group and R₁ is H, alkyl, aryl, or arylalkyl;
with formaldehyde or paraformaldehyde and an amine of formula:

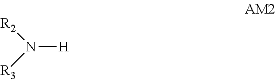

wherein R₂ is C₁₋₄ alkyl and R₃ is C₁₋₄ alkoxy; or R₂ and R₃ taken together with the nitrogen atom to which they are bonded, form a ring selected from:

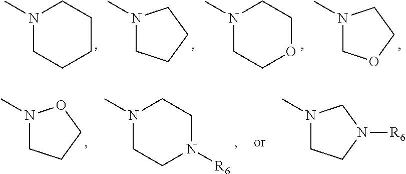

wherein R₆ is H or C₁₋₆ alkyl; in the presence of an acid, optionally acetic acid; and
(b) optionally when the compound of 4′ has R₁═H, the compound may be reacted with an alcohol R₁—OH, to form the compound of formula 4′ wherein R₁ is alkyl, aryl, or arylalkyl.
146. A process according to paragraph 145, wherein:
   Pg is H or an ester or ether OH protecting group; optionally wherein Pg is H, methoxymethyl, tetrahydropyranyl, t-butyl, allyl, benzyl, benzyloxycarbonyl, t-butyldimethylsilyl, t-butyldiphenylsilyl, acetate, acetoxy, pivaloyl, or benzoyl; or wherein Pg is H, benzyl, benzyloxycarbonyl, or acetoxy and/or
   R₁ is H, C₁₋₆ alkyl, C₆₋₁₀ aryl, or C₇₋₁₂ arylalkyl, and optionally wherein R₁ is H or C₁₋₄ alkyl.
147. A process according to paragraph 145 or paragraph 146, wherein:
   Pg is H; and/or
   R₁ is H or C₁₋₄ alkyl.
148. A process according to any of paragraphs 145, 146 or 147, wherein in the amine of formula AM2:
   R₂ is C₁₋₄ alkyl and R₃ is C₁₋₄ alkoxy;
   R₂ is methyl and R₃ is methoxy;
   R₂ and R₃ taken together with the nitrogen atom to which they are bonded, form a ring selected from:

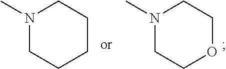

or
R₂ and R₃ taken together with the nitrogen atom to which they are bonded, form the ring:

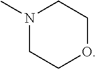

149. A process according to any of paragraphs 145, 146, 147, or 148, wherein R₁ in compound 5′ is H and wherein step (b) is carried out.
150. A process according to any of paragraphs 145, 146, 147, 148, or 149, wherein the reaction is carried out using paraformaldehyde, optionally wherein the acid is acetic acid, or glacial acetic acid.
151. A process for preparing a compound of formula 3′, or a compound of formula 3″, or a mixture of 3′ and 3″:

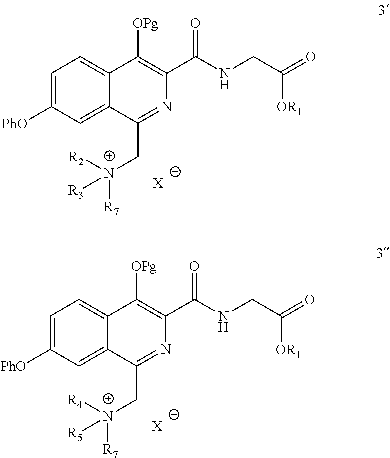

wherein Pg is H or an OH protecting group, R₁ is alkyl, aryl, or arylalkyl; R₂, R₃, R₄, and R₅ each independently represents alkyl, arylalkyl or alkenyl; or R₂ and R₃ and/or R₄ and R₅, taken together with the nitrogen atom to which they are bonded, each independently form a ring selected from:

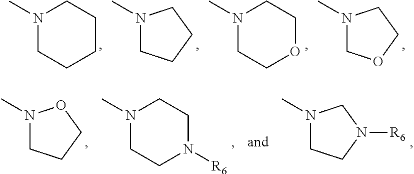

wherein R₆ is H or C₁₋₆ alkyl; R₇ is C₁ to C₆ alkyl and X⁻ is an anion selected from the group consisting of halide, O—SO₄—R₇ wherein R₇ is C₁ to C₆ alkyl, or O—SO₂—R₈ wherein R₈ is phenyl, tolyl, methyl or trifluoromethyl;
comprising reacting a compound of formula 4′, a compound of formula 4″ or a mixture thereof:

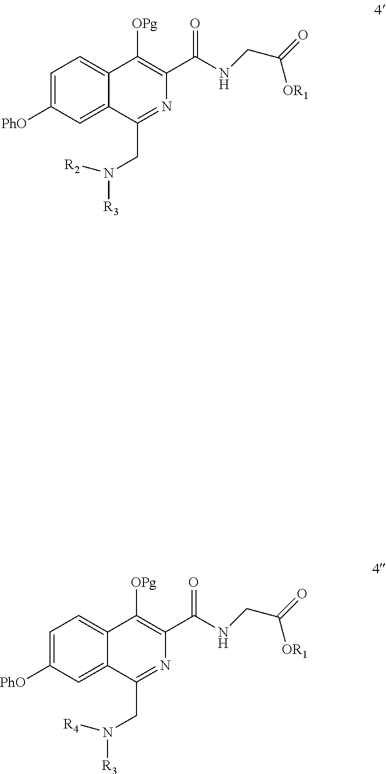

wherein Pg is H or an OH protecting group, R₁ is H, alkyl, aryl, or arylalkyl; or R₂, R₃, R₄, and R₅ each independently represents alkyl, arylalkyl or alkenyl; or R₂ and R₃ and/or R₄ and R₅, taken together with the nitrogen atom to which they are bonded, each independently form a ring selected from:

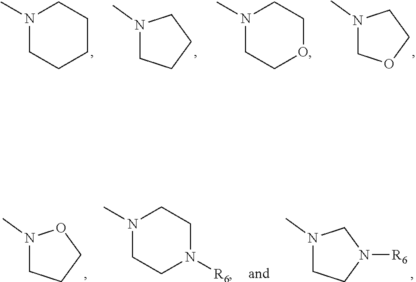

wherein R₆ is H or C₁₋₆ alkyl;
with:
   a compound R₇-Hal, wherein R₇ is C₁ to C₆ alkyl and Hal is halo; or
   a compound of formula R₇O—(SO₂)—OR₇ wherein R₇ is C₁ to C₆ alkyl; or
   a compound of formula R₇O—(SO₂)—R₈, wherein R₇ is C₁ to C₆ alkyl and R₈ is phenyl, tolyl, methyl or trifluoromethyl;
wherein when R₁ in the compound 4′ or 4′″ is H, the resulting compound 3, compound 3″ or a mixture thereof, has R₁═R₇.
152. A process according to paragraph 151 for preparing a compound of formula 3′

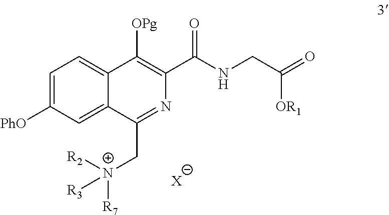

wherein
Pg is H or an OH protecting group;
R₁ is alkyl, aryl, or arylalkyl;
R₂ and R₃, each independently represents alkyl, arylalkyl or alkenyl; or R₂ and
R₃ taken together with the nitrogen atom to which they are bonded, each independently form a ring selected from:

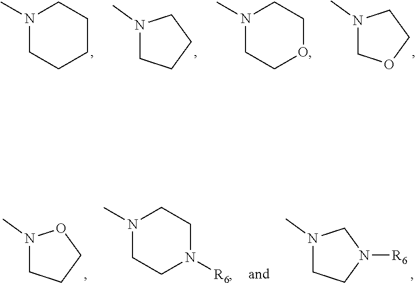

wherein R₆ is H or C₁₋₆ alkyl;
R₇ is C₁ to C₆ alkyl; and
X⁻ is an anion selected from the group consisting of halide, O—SO₄—R₇ wherein
R₇ is C₁ to C₆ alkyl or O—SO₂—R₈ wherein R₈ is phenyl, tolyl, methyl or trifluoromethyl;
comprising reacting a compound of formula 4′:

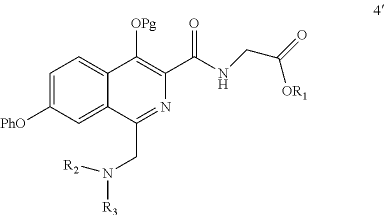

wherein Pg is H or an OH protecting group, R₁ is H, alkyl, aryl, or arylalkyl; R₂ and R₃ each independently represents alkyl, arylalkyl or alkenyl; or R₂ and R₃ taken together with the nitrogen atom to which they are bonded, each independently form a ring selected from:

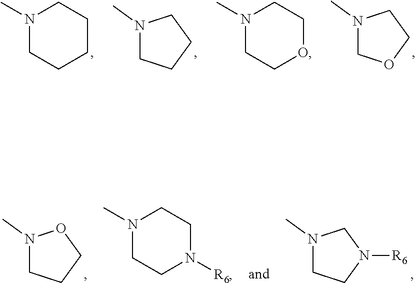

wherein R₆ is H or C₁₋₆ alkyl;
with:
   a compound R₇-Hal, wherein R₇ is C₁ to C₆ alkyl and Hal is halo; or
   a compound of formula R₇O—(SO₂)—OR₇ wherein R₇ is C₁ to C₆ alkyl; or
   a compound of formula R₇O—(SO₂)—R₈, wherein R₇ is C₁ to C₆ alkyl and R₈ is phenyl, tolyl, methyl or trifluoromethyl;
wherein when R₁ in the compound 4′ is H, the resulting compound 3, compound 3″ or a mixture thereof, has R₁═R₇.
153. A process according to paragraph 151 or paragraph 152, wherein:
   Pg is H or an ester or ether OH protecting group; optionally wherein Pg is H, methoxymethyl, tetrahydropyranyl, t-butyl, allyl, benzyl, benzyloxycarbonyl, t-butyldimethylsilyl, t-butyldiphenylsilyl, acetate, acetoxy, pivaloyl, or benzoyl; or wherein Pg is H, benzyl, benzyloxycarbonyl, or acetoxy; and/or R₁ is H, C₁₋₆ alkyl, C₆₋₁₀ aryl, or C₇₋₁₂ arylalkyl, and optionally wherein R₁ is H or C₁₋₄ alkyl;
   Pg is H; and/or
   R₁ is H or C₁₋₄ alkyl.
154. A process according to paragraph 152 or paragraph 153, wherein:
   R₂ and R₃ each independently represents C₁₋₆ alkyl, C₇₋₁₂ arylalkyl or C₂₋₆ alkenyl group; or
   R₂, and R₃ each independently represents C₁₋₆ alkyl; optionally C₁₋₃ alkyl, or wherein R₂ and R₃ are each methyl;
155. A process according to any of paragraphs 152, 153 or 154, wherein R₇ is C₁ to C₃ alkyl; and optionally methyl.
156. A process according to any of paragraphs 152, 153, 154, or 155, wherein X⁻ is an anion selected from the group consisting of chloro, bromo or iodo, or O—SO₄—R₇ wherein R₇ is C₁ to C₃ alkyl, and optionally wherein X⁻ is iodo or O—SO₄-Me.
157. A process according to any of paragraphs 152, 153, 154, 155, or 156, wherein
   the compound R₇-Hal, is a C₁ to C₃ alkyl halide, or methyl halide, or methyl iodide; or
   the compound of formula R₇O—(SO₂)—OR₇ is a di(C₁₋₃ alkyl)sulfate, or dimethylsulfate.
158. A process according to any of paragraphs 152, 153, 154, 155, 156, or 157, wherein the reaction is carried out in an aprotic solvent, optionally wherein the aprotic solvent is a ketone, a nitrile or an ester, optionally wherein the aprotic solvent is acetone, acetonitrile, or ethylacetate, or wherein the reaction is carried out in acetone.
159. A process for preparing a compound of formula 2′:

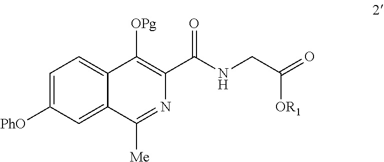

wherein Pg is H or a OH protecting group, and R₁ is H, alkyl, aryl, or arylalkyl, comprising:
   reducing a compound of formula 3′, a compound of formula 3′ or a mixture thereof:

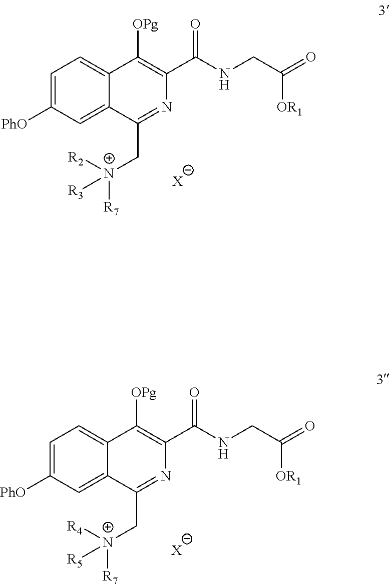

wherein Pg is an H or an OH protecting group, R₁ is alkyl, aryl, or arylalkyl; R₂, R₃, R₄, and R₅ each independently represents alkyl, aryl, arylalkyl or alkenyl; or R₂ and R₃ and/or R₄ and R₅, taken together with the nitrogen atom to which they are bonded, each independently form a ring selected from:

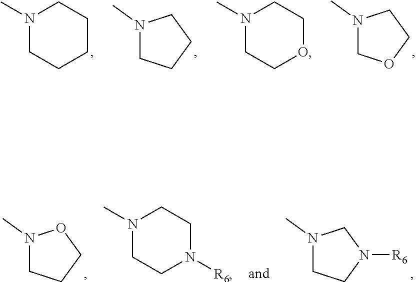

wherein R₆ is H or C₁₋₆ alkyl; R₇ is C₁ to C₆ alkyl; and X⁻ is an anion selected from halide, O(SO₄)R₇ wherein R₇ is C₁ to C₆ alkyl, or O—SO₂—R₈ wherein R₈ is phenyl, tolyl, methyl or trifluoromethyl;
or
   reducing a compound of formula 4′, a compound of formula 4″ or a mixture thereof

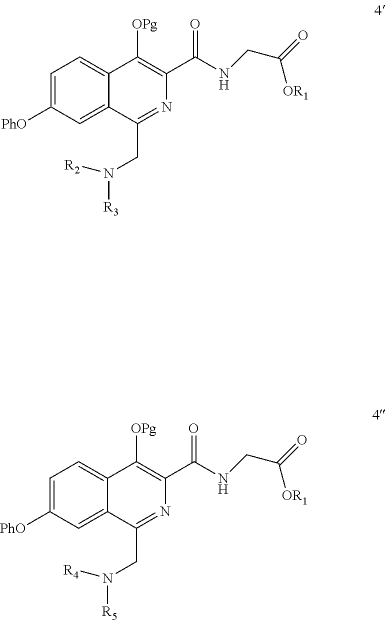

wherein Pg is H or an OH protecting group, R₁ is H, alkyl, aryl, or arylalkyl; R₂, R₃, R₄, and R₅ each independently represents alkyl, aryl, arylalkyl or alkenyl; or R₂ is C₁₋₄ alkyl and R₃ is C₁₋₄ alkoxy; or R₂ and R₃ and/or R₄ and R₅ taken together with the nitrogen atom to which they are bonded, each independently form a group selected from:

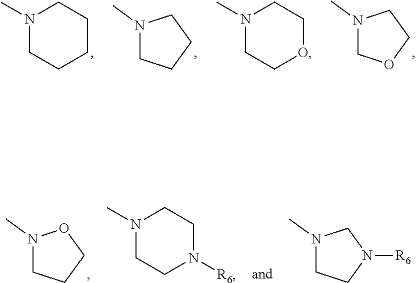

wherein R₆ is H or C₁₋₆ alkyl.
160. A process according to paragraph 90 for preparing a compound of formula 2′:

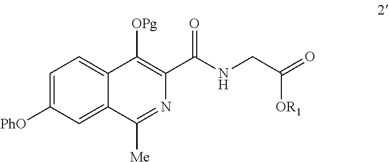

wherein Pg is H, or a OH protecting group, and R₁ is H, alkyl, aryl, or arylalkyl, comprising reducing a compound of formula 3′:

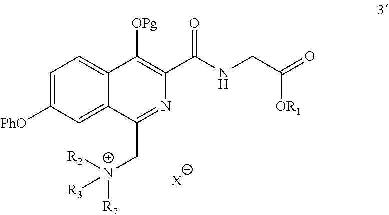

wherein Pg is an H or a OH protecting group;
R₁ is alkyl, aryl, or arylalkyl;
R₂ and R₃, each independently represents alkyl, aryl, arylalkyl or alkenyl; or R₂ and R₃ taken together with the nitrogen atom to which they are bonded, each independently form a ring selected from:

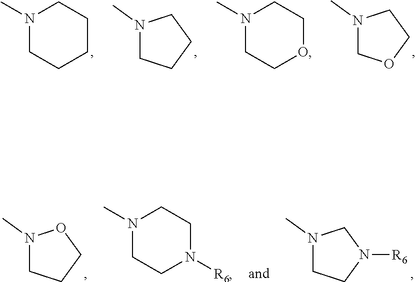

wherein R₆ is H or C₁₋₆ alkyl;
R₇ is C₁ to C₆ alkyl; and
X⁻ is an anion selected from halide, O(SO₄)R₇ wherein R₇ is C₁ to C₆ alkyl, or O—SO₂—R₈ wherein R₈ is phenyl, tolyl, methyl or trifluoromethyl.
161. A process according to paragraph 160, wherein:
   Pg is H or an ester or ether OH protecting group; optionally wherein Pg is H, methoxymethyl, tetrahydropyranyl, t-butyl, allyl, benzyl, benzyloxycarbonyl, t-butyldimethylsilyl, t-butyldiphenylsilyl, acetate, acetoxy, pivaloyl, or benzoyl; or wherein Pg is H, benzyl, benzyloxycarbonyl, or acetoxy, and/or
   R₁ is H, C₁₋₆ alkyl, C₆₋₁₀ aryl, or C₇₋₁₂ arylalkyl, and optionally wherein R₁ is H or C₁₋₄ alkyl; or
   Pg is H; and/or
   R₁ is H or C₁₋₄ alkyl.
162. A process according to paragraph 160 or paragraph 161, wherein:
   R₂ and R₃ each independently represents C₁₋₆ alkyl, C₇₋₁₂ arylalkyl or C₂₋₆ alkenyl group; or
   R₂, and R₃ each independently represents C₁₋₆ alkyl; optionally C₁₋₃ alkyl, or wherein R₂ and R₃ are each methyl.
163. A process according to any of paragraphs 160, 161 or 162, wherein R₇ is C₁ to C₃ alkyl; and optionally methyl.
164. A process according to any of paragraphs 160, 161, 162, or 163, wherein X⁻ is an anion selected from the group consisting of chloro, bromo or iodo, or O—SO₄—R₇ wherein R₇ is C₁ to C₃ alkyl, and optionally wherein X⁻ is iodo or O—SO₄-Me.
165. A process according to any of paragraphs 160, 161, 162, 163, or 164, wherein the reduction is carried out using:
   zinc and an acid, optionally zinc and acetic acid; or
   hydrogen gas and Pd/catalyst.
166. A process according to paragraph 165, wherein the reduction is carried out in a polar solvent, wherein the polar solvent is optionally an alcohol, a ketone or an ester, and optionally a ketone, or wherein the polar solvent is acetone.
167. A process according to paragraph 90 for preparing a compound of formula 2′:

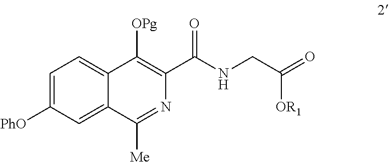

wherein Pg is an OH protecting group, and R₁ is H, alkyl, aryl, or arylalkyl, comprising reducing a compound of formula 4′:

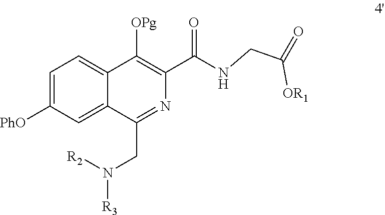

wherein Pg is an OH protecting group, R₁ is H, alkyl, aryl, or arylalkyl; R₂, R₃, R₄, and R₅ each independently represents alkyl, aryl, arylalkyl or alkenyl; or R₂ is C₁₋₄ alkyl and R₃ is C₁₋₄ alkoxy; or R₂ and R₃ and/or R₄ and R₅ taken together with the nitrogen atom to which they are bonded, each independently form a group selected from:

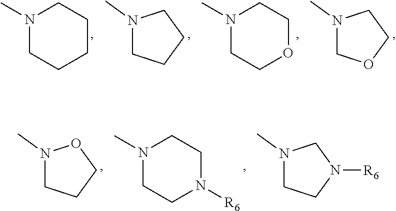

wherein R₆ is H or C₁₋₆ alkyl.
168. A process according to paragraph 167, wherein:
   Pg is H or an ester or ether OH protecting group; optionally wherein Pg is H, methoxymethyl, tetrahydropyranyl, t-butyl, allyl, benzyl, benzyloxycarbonyl, t-butyldimethylsilyl, t-butyldiphenylsilyl, acetate, acetoxy, pivaloyl, or benzoyl; or wherein Pg is H, benzyl, benzyloxycarbonyl, or acetoxy, and/or
   R₁ is H, C₁₋₆ alkyl, C₆₋₁₀ aryl, or C₇₋₁₂ arylalkyl, and optionally wherein R₁ is H or C₁₋₄ alkyl; or
   Pg is H; and/or
   R₁ is H or C₁₋₄ alkyl.
169. A process according to paragraph 167 and 168, wherein R₂ and R₃ each independently represents C₁₋₆ alkyl, C₇₋₁₂ arylalkyl or C₂₋₆ alkenyl group.
70. A process according to any of paragraphs 167, 168 or 169, wherein R₂, and R₃ each independently represents C₁₋₆ alkyl; optionally C₁₋₃ alkyl, or wherein R₂ and R₃ are each methyl.
171. A process according to any of paragraphs 167, 168, 169, or 170, wherein the reduction is carried out using:
   zinc and an acid, optionally zinc and hydrochloric acid, or zinc and concentrated hydrochloric acid; or
   hydrogen gas and Pd/catalyst.
172. A process according to paragraph 171, wherein the reduction is carried out in a polar solvent, wherein the polar solvent is optionally a chlorinated hydrocarbon, a ketone or an ester, and optionally a chlorinated C₁₋₃ alkane, or wherein the polar solvent is dichloromethane.
173. A process according to any of paragraphs 125-172, further comprising converting the compound of formula 5′; the compound of formula 4′, the compound of formula 4″ or a mixture of the compounds of formula 4′ and 4″; the compound of formula 3′, the compound of formula 3″ or a mixture of the compounds of formula 3 and 3″, to Roxadustat or a salt thereof.
   In a further aspect, the disclosure relates to compounds which may be used as intermediates in the synthesis of Roxadustat, for example according to any of the processes described herein. Thus, the disclosure provides:
174. A compound of:
   (i) formula 3′:

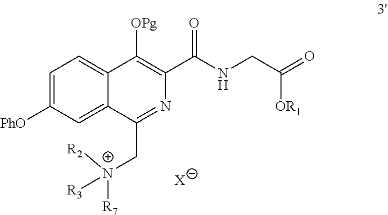

wherein:
Pg is H or an OH protecting group;
R₁ is C₁₋₆ alkyl, C₆₋₁₀ aryl, or C₇₋₁₂ arylalkyl;
R₂ and R₃, each independently represents C₁₋₆ alkyl, C₇₋₁₂ arylalkyl or C₂₋₆ alkenyl; or
R₂ and R₃ taken together with the nitrogen atom to which they are bonded, each independently form a ring selected from:

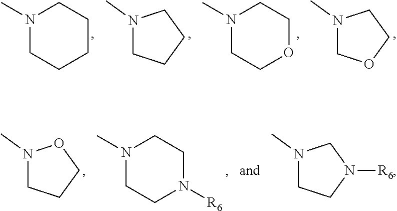

wherein R₆ is H or C₁₋₆ alkyl;
R₇ is C₁ to C₆ alkyl; and
and X⁻ is an anion selected from the group consisting of halide, O—SO₄—R₇ wherein R₇ is C₁ to C₆ alkyl, or O—SO₂—R₈ wherein R₈ is phenyl, tolyl, methyl or trifluoromethyl; or
(ii) formula 4′:

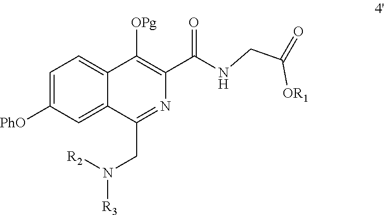

wherein:
Pg is H or an OH protecting group;
R₁ is H, C₁₋₆ alkyl, C₆₋₁₀ aryl, or C₇₋₁₂ arylalkyl;
R₂ and R₃ each independently represents C₁₋₆ alkyl, C₆₋₁₀ aryl, C₇₋₁₂ arylalkyl or C₂₋₆ alkenyl; or
R₂ is C₁₋₄ alkyl and R₃ is C₁₋₄ alkoxy; or
R₂ and R₃ taken together with the nitrogen atom to which they are bonded, each independently form a ring selected from:

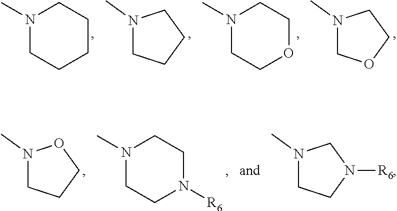

wherein R₆ is H or C₁₋₆ alkyl; or
(iii) formula 5:

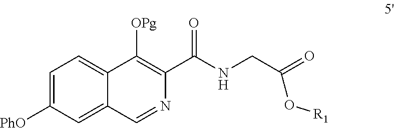

wherein:
Pg is H, or an OH protecting group; and
R₁ is H, C₁₋₆ alkyl, C₆₋₁₀ aryl or C₇₋₁₂ arylalkyl.
175. A compound according to paragraph 174 of:
   (i) formula 3′:

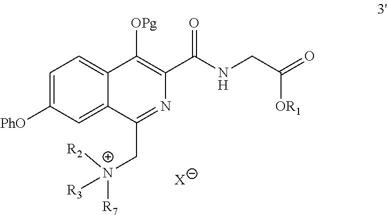

wherein:
Pg is H, methoxymethyl, tetrahydropyranyl, t-butyl, allyl, benzyl, benzyloxycarbonyl, t-butyldimethylsilyl, t-butyldiphenylsilyl, acetate, acetoxy, pivaloyl, or benzoyl; or Pg is selected from H, benzyl, benzyloxycarbonyl or acetoxy;
R₁ is C₁₋₃ alkyl;
R₂ and R₃ each independently represents C₁₋₃ alkyl; or
R₂ and R₃ taken together with the nitrogen atom to which they are bonded, each independently form a ring selected from:

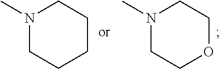

R₇ is C₁ to C₃ alkyl; and
X⁻ is an anion selected from the group consisting of halide or O—SO₄—R₇
wherein R₇ is C₁ to C₃ alkyl; or
(ii) formula 4′:

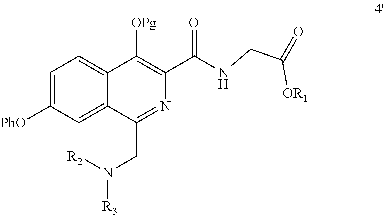

wherein Pg is H, methoxymethyl, tetrahydropyranyl, t-butyl, allyl, benzyl, benzyloxycarbonyl, t-butyldimethylsilyl, t-butyldiphenylsilyl, acetate, acetoxy, pivaloyl, or benzoyl; or Pg is selected from H, benzyl, benzyloxycarbonyl or acetoxy;
R₁ is H or C₁₋₃ alkyl; and
R₂ and R₃, each independently represents C₁₋₃ alkyl; or
R₂ is C₁₋₃ alkyl and R₃ is C₁₋₃ alkoxy; or
R₂ and R₃, taken together with the nitrogen atom to which they are bonded, each independently form a ring selected from:

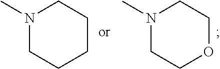

or
(iii) formula 5:

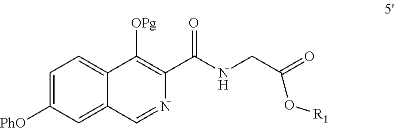

wherein:
Pg is H, methoxymethyl, tetrahydropyranyl, t-butyl, allyl, benzyl, benzyloxycarbonyl, t-butyldimethylsilyl, t-butyldiphenylsilyl, acetate, acetoxy, pivaloyl, or benzoyl; or Pg is selected from H, benzyl, benzyloxycarbonyl or acetoxy; and
R₁ is H or C₁₋₃ alkyl.
176. A compound according to paragraph 175 of:
   (i) formula 3′:

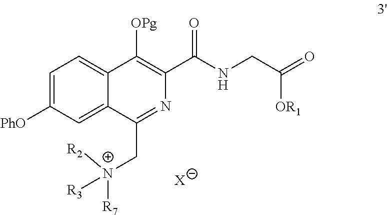

wherein:
Pg is H, benzyl, benzyloxycarbonyl, or acetoxy;
R₁ is methyl or ethyl;
R₂ and R₃ are each methyl; or
R₂ and R₃ taken together with the nitrogen atom to which they are bonded, each independently form a ring selected from:

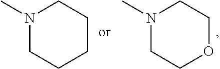

and
R₇ is methyl; and
and X⁻ is an anion selected from the group consisting of chloro, bromo or iodo, or O—SO₄—R₇ wherein R₇ is C₁ to C₃ alkyl; or
(ii) formula 4′:

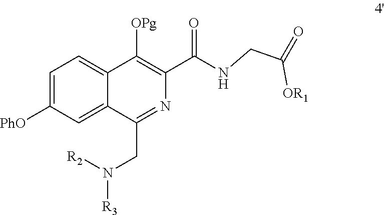

wherein Pg is H, benzyl, benzyloxycarbonyl, or acetoxy;
R₁ is H, methyl or ethyl; or
R₂ is methyl; R₃ is methoxy; or
R₂ and R₃, each independently represents methyl; or R₂ and R₃, taken together with the nitrogen atom to which they are bonded, each independently form a ring selected from:

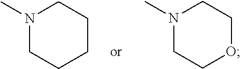

or
(iii) formula 5:

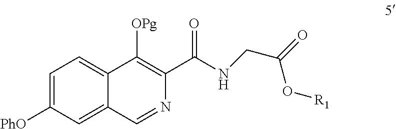

wherein:
Pg is H, benzyl, benzyloxycarbonyl, or acetoxy; and
R₁ is H, methyl or ethyl.
177. A compound according to paragraph 176 of:
   (i) formula 3′:

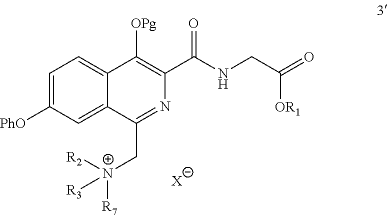

wherein:
Pg is H;
R₁ is H, methyl or ethyl;
R₂ and R₃ are each methyl; or
R₂ and R₃ taken together with the nitrogen atom to which they are bonded, each independently form a ring selected from:

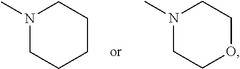

and
R₇ is methyl; and
X⁻ is an anion selected from the group consisting of iodo, or O—SO₄-Me; or
(ii) formula 4′:

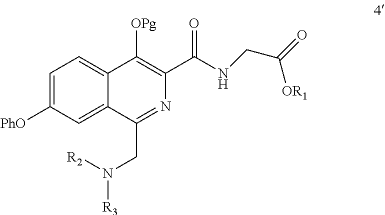

wherein Pg is H;
R₁ is H, methyl or ethyl;
R₂ and R₃ are each methyl;
R₂ is methyl and R₃ is methoxy;
or R₂ and R₃, taken together with the nitrogen atom to which they are bonded, each independently form a ring selected from:

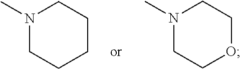

or
(iii) formula 5:

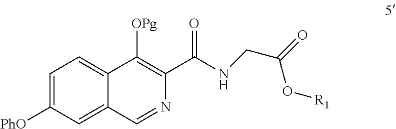

wherein:
Pg is H; and
R₁ is H, methyl or ethyl.
178. A compound according to paragraph 177 selected from the group consisting of:

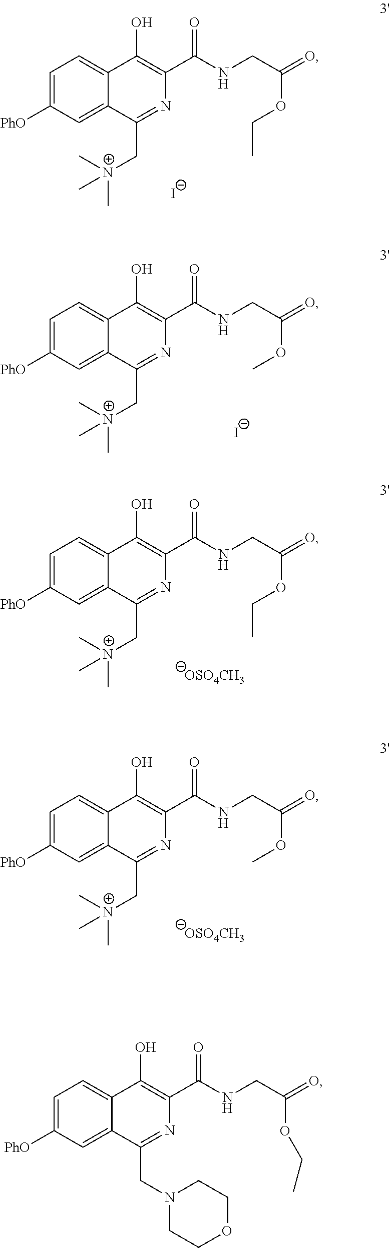

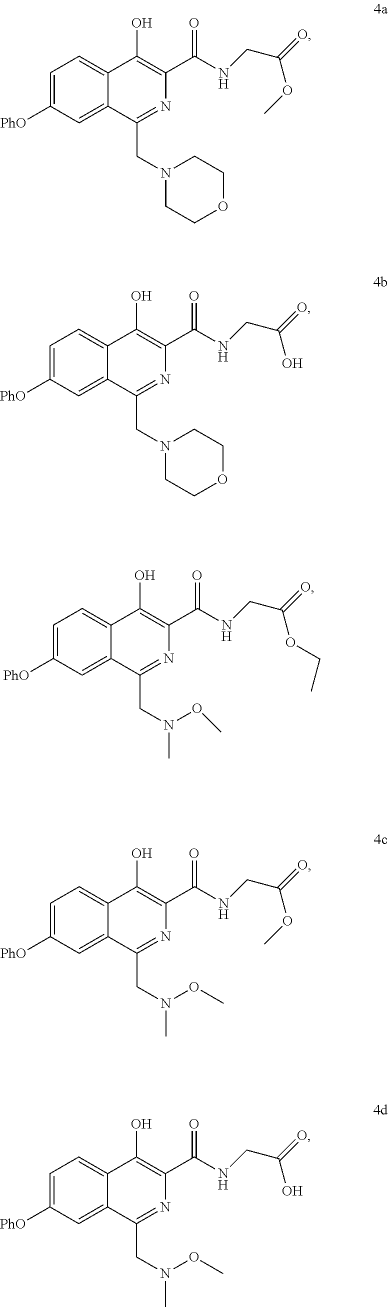 [SMILES: COC(=O)CNC(=O)c1nc(CN2CCOCC2)c2cc(Oc3ccccc3)ccc2c1O ; O=C(O)CNC(=O)c1nc(CN2CCOCC2)c2cc(Oc3ccccc3)ccc2c1O ; CCOC(=O)CNC(=O)c1nc(CN(C)OC)c2cc(Oc3ccccc3)ccc2c1O ; COC(=O)CNC(=O)c1nc(CN(C)OC)c2cc(Oc3ccccc3)ccc2c1O ; CON(C)Cc1nc(C(=O)NCC(=O)O)c(O)c2ccc(Oc3ccccc3)cc12]

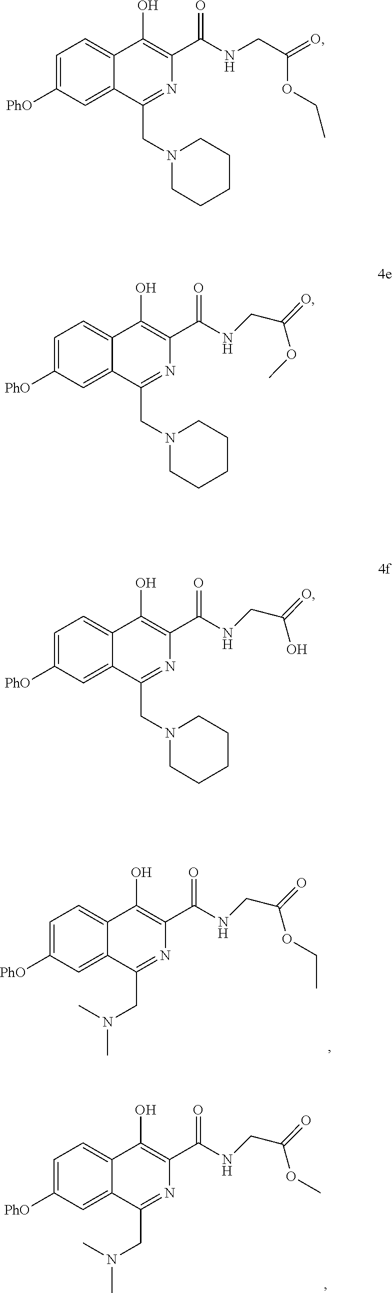 [SMILES: CCOC(=O)CNC(=O)c1nc(CN2CCCCC2)c2cc(Oc3ccccc3)ccc2c1O ; COC(=O)CNC(=O)c1nc(CN2CCCCC2)c2cc(Oc3ccccc3)ccc2c1O ; O=C(O)CNC(=O)c1nc(CN2CCCCC2)c2cc(Oc3ccccc3)ccc2c1O ; CCOC(=O)CNC(=O)c1nc(CN(C)C)c2cc(Oc3ccccc3)ccc2c1O ; COC(=O)CNC(=O)c1nc(CN(C)C)c2cc(Oc3ccccc3)ccc2c1O]

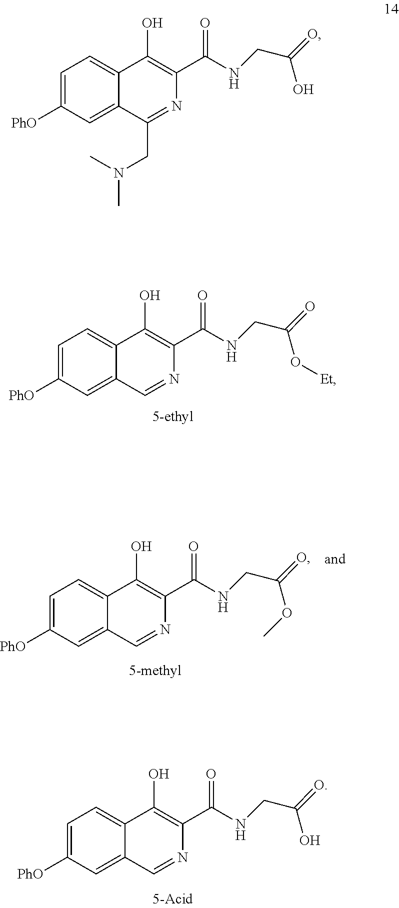 [SMILES: CN(C)Cc1nc(C(=O)NCC(=O)O)c(O)c2ccc(Oc3ccccc3)cc12 ; CCOC(=O)CNC(=O)c1ncc2cc(Oc3ccccc3)ccc2c1O ; COC(=O)CNC(=O)c1ncc2cc(Oc3ccccc3)ccc2c1O ; O=C(O)CNC(=O)c1ncc2cc(Oc3ccccc3)ccc2c1O]

179. A compound according to paragraph 178 selected from the group consisting of:

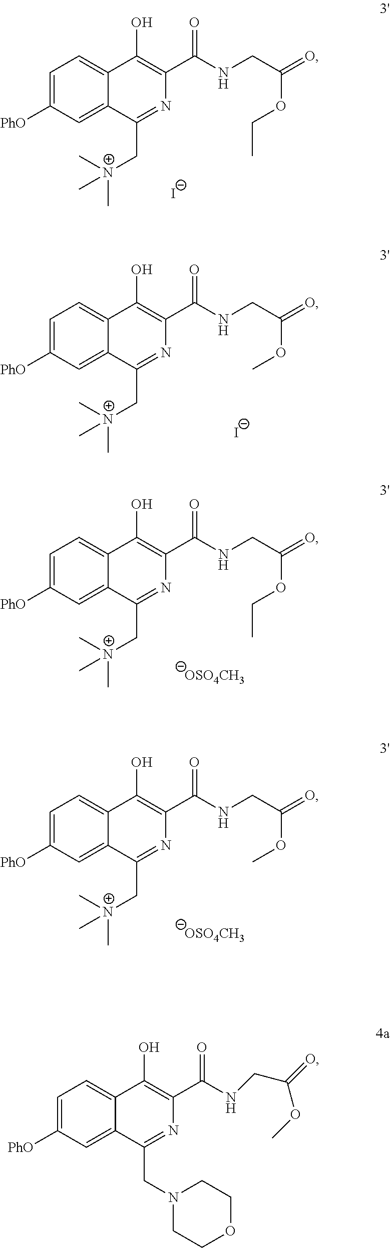

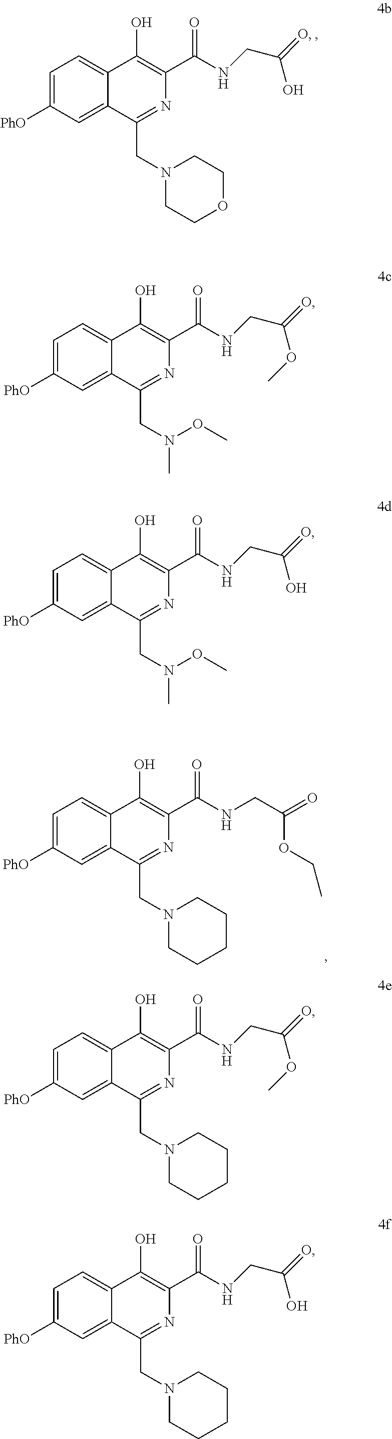

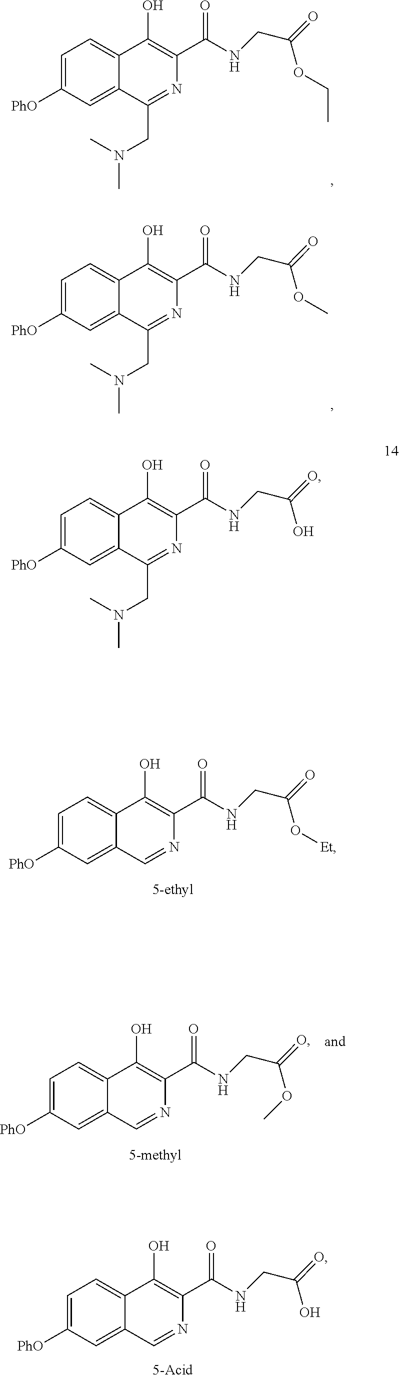 [SMILES: CCOC(=O)CNC(=O)c1nc(CN(C)C)c2cc(Oc3ccccc3)ccc2c1O ; COC(=O)CNC(=O)c1nc(CN(C)C)c2cc(Oc3ccccc3)ccc2c1O ; CN(C)Cc1nc(C(=O)NCC(=O)O)c(O)c2ccc(Oc3ccccc3)cc12 ; CCOC(=O)CNC(=O)c1ncc2cc(Oc3ccccc3)ccc2c1O ; COC(=O)CNC(=O)c1ncc2cc(Oc3ccccc3)ccc2c1O ; O=C(O)CNC(=O)c1ncc2cc(Oc3ccccc3)ccc2c1O]

A further aspect of the disclosure provides the use of these compounds, especially the compounds of any of paragraphs 177, 178 and 179, for preparing Roxadustat or a salt thereof.

In further aspect Roxadustat prepared according to any process of the present disclosure is substantially pure.

In further aspect Roxadustat prepared according to any process of the present disclosure is substantially pure wherein at least about more than 99%, more than about 99.5% or more than about 99.8% Roxadustat is measured by any technique described in the literature. e.g. by HPLC.

The present disclosure also encompasses the use of the Roxadustat prepared by the any of the processes of the present disclosure for the preparation of pharmaceutical compositions of Roxadustat.

The present disclosure comprises processes for preparing the above mentioned pharmaceutical compositions. The processes comprise combining the Roxadustat prepared by any of the processes of the present disclosure with at least one pharmaceutically acceptable excipient.

Roxadustat prepared by any of the processes of the present disclosure and the pharmaceutical compositions of Roxadustat prepared by any processes of the present disclosure can be used as medicaments, particularly for the treatment of anemia.

The present disclosure also provides methods for the treatment of anemia, comprising administering a therapeutically effective amount of Roxadustat prepared by any of the processes of the present disclosure, or at least one of the above pharmaceutical compositions, to a subject in need of the treatment.

### EXAMPLES

### Example 1: Preparation of 4-hydroxy-7-phenoxyisoquinoline-3-carboxylic Acid (Formula 6) via Formula 7

Methyl 4-hydroxy-7-phenoxyisoquinoline-3-carboxylate (Compound 7, 50 gm, 0.169 moles) was added to the reactor followed by addition of the THF (150 ml) and Ethanol (250 ml). Reaction mass was stirred at 20-30° C. for 20-30 min and added Aq. Sodium Hydroxide solution (28 gm, 0.7 moles in 150 ml water) in drop wise to the reaction mass at 25-55° C. Reaction mass was maintained under stirring for 4-7 hrs. The reaction was monitored by HPLC. Reaction mass was acidified using conc. HCl up to pH 1-2, water (1500 ml) was added to the reaction mass and stirred for 20-30 min and filtered the reaction mass. The compound was dried under vacuum at 55° C. and material was forwarded to next stage without further purification. The yield obtained was 85-90% and HPLC purity >95%.

### Example 2: Preparation of ethyl (4-hydroxy-7-phenoxyisoquinoline-3-carbonyl)glycinate (Formula 5) Via Formula 6

4-hydroxy-7-phenoxyisoquinoline-3-carboxylic acid (Compound 6, 45.0 gm, 0.159 moles) was added to the reactor at 20-30° C. MDC (360 ml) was added to the reaction mass followed by Glycine ethyl ester hydrochloride (44.51 gm, 0.31 mole). Tri ethyl amine (113 ml) and propanephosphonic acid anhydride (152.77 gm, 0.480 moles) was added subsequently. The reaction mass was heated up to 50-60° C. for 4-8 hrs. The reaction mass was monitored by HPLC. After completion of reaction, D.I. water (225 ml) was added to the reaction mass, the layers were separated and organic layer was washed with D. I. Water (90 ml). The organic layer was evaporated under reduced pressure to get crude compound, which was purified by mixture of ethyl acetate (45 ml) and cyclohexane (450 ml), the reaction mass was filtered and washed by cyclohexane (50 ml). The compound was dried under vacuum at 45° C. The yield obtained was 92-98% and HPLC purity >93%.

### Example 3: Preparation of ethyl (1-((dimethylamino)-methyl)-4-hydroxy-7-phenoxyiso-quinoline-3-carbonyl)glycinate (Compound 4) Via Formula 5

Ethyl (4-hydroxy-7-phenoxyisoquinoline-3-carbonyl)glycinate (compound 5 55.0 gm, 0.150 moles) was charged in acetic acid (165 ml) into reactor. Added N,N,N′,N′-Tetramethylmethanediamine (28 ml) into the reaction mass at 20-30° C. in 10-20 min. Heated the reaction mass to 60-70° C. and stirred it for 6-10 hrs. The reaction mass was monitored by HPLC. After completion of reaction, reaction mass was cooled to 20-30° C. and charged ethyl acetate (300 ml) and water (200 ml) in to it. Stirred the mass and separated the organic layer. Organic layer was washed with aq. sodium carbonate solution. Organic layer was collected and was evaporated under reduced pressure to get crude compound. The yield obtained was 85-90% and HPLC purity >96%

### Example 4a: Preparation of 1-(3-((2-ethoxy-2-oxoethyl)-carbamoyl)-4-hydroxy-7-phenoxy-isoquinolin-1-yl)-N,N,N-trimethylmethanaminium iodide (Formula 3-iodide) Via Formula 4

Ethyl-(1-((dimethylamino)methyl)-4-hydroxy-7-phenoxyisoquinoline-3-carbonyl)-glycinate (compound 4.8 gm, 0.0189 moles) was charged in acetone (100 ml) in to the reactor. Reaction mass stirred at 20-30° C. for 30 min and added methyl iodide (3.20 gm, 0.022 moles) drop wise in to the reaction mass at 20-30° C. The reaction mass was maintained under stirring at 20-30° C. for 6-12 hour. The reaction was monitored by HPLC. After completion of reaction, reaction mass was filtered and washed the solid by acetone (24 ml). Wet solid was dried under vacuum at 45-55° C. for 5-15 hours. The yield obtained is 90-95% and HPLC Purity is >95%.

### Example 4b. Preparation of 1-(3-((2-ethoxy-2-oxoethyl)-carbamoyl)-4-hydroxy-7-phenoxy-isoquinolin-1-yl)-N,N,N-trimethylmethanaminium O-methyl sulfuroperoxoate ((Formula 3)-O-methyl sulfuroperoxoate) Via Formula 4

Ethyl-(1-((dimethylamino)methyl)-4-hydroxy-7-phenoxyisoquinoline-3-carbonyl)-glycinate (compound 4, 15 gm, 0.035 moles) was charged in acetone (120 ml) in to the reactor. Reaction mass stirred at 20-30° C. for 30 min and added DMS (5.36 gm, 0.042 moles) drop wise in to the reaction mass at 20-30° C. The reaction mass was maintained under stirring at 20-30° C. for 6-12 hour. The reaction was monitored by HPLC. After completion of reaction, reaction mass was filtered and washed the solid by acetone (30 ml). Wet solid was dried under vacuum at 45-55° C. for 5-15 hours. The yield obtained is 90-95% and HPLC Purity is >93%.

### Example 5a: Preparation of ethyl (4-hydroxy-1-methyl-7-phenoxyisoquinoline-3-carbonyl) glycinate (Formula 2) Via Formula 3

Charged 1-(3-((2-ethoxy-2-oxoethyl)carbamoyl)-4-hydroxy-7-phenoxyisoquinolin-1-yl)-N,N,N-trimethylmethanaminium iodide (3-Iodid 5 gm, 0.0088 moles) in to mixture of acetic acid (125 ml) and water (50 ml). Charged the acetone (50 ml) in to the reaction mass and heated to 50-60° C. Maintained the reaction mass for 30-60 min. zinc dust (2.88 gm, 0.044 moles) was charged in to the reaction mass and stirred reaction mass at 50-60° C. for 3-8 hours. The reaction was monitored by HPLC. After completion of reaction, reaction mass was cooled to 20-30° C. and charged reaction mass in mixture of dichloromethane (200 ml) and water (200 ml) in another reactor. Reaction mass was stirred for 1-2 hour at 20-30° C. Layers were separated at 20-30° C. Organic layer was collected and washed the organic layer with 4% aq. sodium carbonate solution (200 ml) and distilled the organic layer under vacuum at 45-55° C. Acetone (25 ml) was charged in to the residue at 20-30° C. Reaction mass was heated to 50-56° C. and stirred for 30-120 min. The reaction mass was cooled slowly to 20-30° C. and stirred for 1-3 hour at 20-30° C. Water (25 ml) was added (25 ml) in to the reaction mass at 20-30° C. in 20-30 min. Stirred the reaction mass for 2-4 hour and filtered. Wet cake washed with 20 ml (1:1) mixture of acetone and water. The wet compound dried under vacuum at 45-55° C. The yield obtained is 80-90% and HPLC Purity is >99%

### Example 5b: Preparation of ethyl (4-hydroxy-1-methyl-7-phenoxyisoquinoline-3-carbonyl) glycinate (Formula 2) Via Formula 3

1-(3-((2-ethoxy-2-oxoethyl)carbamoyl)-4-hydroxy-7-phenoxyisoquinolin-1-yl)-N,N,N-trimethylmethanaminium iodide (3-Iodid 1 gm, 0.0017 moles) charged in to mixture of acetone (40 ml) and water (10 ml). Stirred the mass at 30-40° C. for 30-60 min. Charged clear reaction mass in to autoclave at 20-30° C. followed by charging of 5% Pd/C (0.6 gm). Heated reaction mass to 50-70° C. and stirred the reaction mass at 50-70° C. and under 5-15 Kg/cm2 of hydrogen gas pressure for 8-16 hours. Reaction mass was cooled to 20-30° C. and released the hydrogen pressure. Reaction mass was filtered through the hyflo bed followed by washing of 30 ml (1:1) mixture of acetone and water. Solvent distilled out form the filtrate at 45-55° C. under vacuum. Charged ethyl acetate (25 ml) and water (15 ml) to the residue and stirred the reaction mass for 20-30 min at 20-30° C. Layers were separated and organic layer was distilled out under vacuum at 40-50° C. The yield obtained was 60% and HPLC Purity >91%.

### Example 6: Preparation of (4-hydroxy-1-methyl-7-phenoxyisoquinoline-3-carbonyl) glycine (Formula 1-Roxadustat) Via Formula 2

Ethyl (4-hydroxy-1-methyl-7-phenoxyisoquinoline-3-carbonyl)glycinate (compound 2 2.8 gm, 0.0073 moles) was charged in to the reactor followed by the addition of THF (30 ml). Reaction mass was stirred for 10-15 min at 20-30° C. The solution of Sodium hydroxide (0.74 gm, 0.0185 moles) in water (20 ml) and Ethanol (20 ml) was charged in to the reaction mass at 20-30° C. The reaction mass was heated to 55-65° C. and stirred the mass for 2-8 hours. The reaction was monitored by HPLC. After completion of reaction, solvent was distilled out form the reaction mass under vacuum at temperature 45-55° C. Charged water (40 ml) and Ethyl acetate (30 ml) in to the residue at 20-30° C. and stirred it for 30-40 min at 20-30° C. Layers were separated and aqueous layer was collected and added conc. HCl (3.5 ml) in to the aqueous layer at 20-30° C. reaction mass was stirred the at 20-30° C. for 2-5 hours. Filtered the reaction mass at 20-30° C. followed by the water (30 ml) wash. Wet solid was charged in to the reactor followed by charging of Ethanol (50 ml) at 20-30° C. Heated the mass to 70-80° C. and stirred the mass at 70-80° C. for 1-3 hour. Cooled the mass to 20-30° C. and stirred the mass for 2-3 hour. Filtered the reaction mass followed by the ethanol (20 ml) wash. Wet solid was charged in to the reactor followed by charging of methanol (30 ml). Heated the reaction mass to 60-65° C. and stirred for 2-3 hour. Cooled the reaction mass to 20-30° C. and stirred the mass for 2-3 hour. Filtered the mass at 20-30° C. followed by the methanol (20 ml) wash. The wet compound dried under vacuum at 50° C. The yield obtained is 73.8% Roxadustat and HPLC Purity is >99%.

### Example 7: Preparation of (4-hydroxy-7-phenoxyisoquinoline-3-carbonyl) glycine (Formula 5-acid) Via Formula 7

Methyl 4-hydroxy-7-phenoxyisoquinoline-3-carboxylate (Compound 7, 25.0 gm, 0.08 moles) was added to the reactor at 20-30° C. Methanol (350 ml) was added to the reaction mass followed by Glycine (31.72 gm, 0.31 mole) and Sodium methoxide (13.71 gm 0.58 mole). The reaction mass was heated to 80-90° C. for 8-10 hrs. The reaction mass was monitored by HPLC. After completion of reaction, reaction mass was filtered and dried over suction, the wet cake was dissolved in water (225 ml) & Ethyl acetate (125 ml). The layers were separated and acidify the aqueous layer with conc HCl up to pH 1-2. The Ethyl acetate (125 ml) was added to the aq. Layer and layers were separated, organic layer was collected and was evaporated under reduced pressure to get crude (4-hydroxy-7-phenoxyisoquinoline-3-carbonyl) glycine. The yield obtained was 92-98% and HPLC purity >98%.

### Example 8: Preparation of (1-((dimethylamino)methyl)-4-hydroxy-7-phenoxyisoquioline-3-carbonyl)glycine (Formula 14) Via Formula (5-acid)

(4-hydroxy-7-phenoxyisoquinoline-3-carbonyl)glycine (Compound 5-acid 5 gm, 0.0147 moles) was charged in to acetic acid (15 ml) in reactor. Added tetramethyldiaminomethane (1.88 gm, 0.0183 moles) in to reaction mass at 20-30° C. in 10-20 min. Reaction mass was heated to 55-65° C. and stirred for 5-15 hrs at 55-65° C. After completion of reaction, reaction mass was cooled to 20-30° C. and charged ethyl acetate (125 ml) and water (75 ml) in to the reaction mass. Reaction mass was stirred at 20-30° C. for 30-40 min. Layers were separated and charged ethyl acetate (100 ml) in to the organic layer at 20-30° C. and stirred reaction mass at 20-30° C. for 2-4 hour. Filtered the reaction mass followed by washing of ethyl acetate (20 ml). The wet compound was dried under vacuum at 50° C. The yield obtained is 48% and HPLC Purity is >99%.

### Example 9: Preparation of ethyl (1-((dimethylamino)methyl)-4-hydroxy-7-phenoxy-iso-quinoline-3-carbonyl)glycinate (Formula 4) with Formula 14

(1-((dimethylamino)-methyl)-4-hydroxy-7-phenoxyisoquinoline-3-carbonyl)glycine (compound 14, 2 gm, 0.005 moles) was charged in to Ethanol (40 ml) at 20-30° C. Sulfuric acid (0.5 gm) was charged at 20-30° C. The reaction mass was heated to 75-80° C. and stirred for 4-12 hour at 75-80° C. The reaction was monitored by HPLC. After completion of reaction, the solvent was distilled out from reaction mass at 50° C. Charged ethyl acetate (40 ml) and 10% sodium carbonate solution (30 ml) in to residue and stirred the reaction mass for 20-30 min. Layers were separated and organic layer was collected. Evaporate the organic layer under vacuum at 45-55° C. Charged cyclohexane (40 ml) in to residue and stirred it for 2-4 hour. Reaction mass was filtered and washed using cyclohexane (10 ml). The wet compound was dried under vacuum at 50° C. The yield obtained is 86.9% and HPLC Purity is 93%.

### Example 10: Preparation of methyl (4-hydroxy-7-phenoxyisoquinoline-3-carbonyl)-glycinate (Formula 5 methyl) Via Formula (5-acid)

(4-hydroxy-7-phenoxyisoquinoline-3-carbonyl)glycine (compound5-acid, 5.5 gm, 0.160 moles) was added to the reactor at 20-30° C. Methanol (100 ml) and Sulfuric acid (1.0 ml) was added to the reaction mass at 20-30° C. Temperature of Reaction mass was raised 50-65° C. and maintained at 50-65° C. for 7-8 hrs. The reaction mass was monitored by HPLC. After completion of reaction, charged D.M. water (50 ml) and Ethyl acetate (300 ml) to the reaction mass. The reaction mass was monitored by HPLC. The yield obtained was 95-98% and HPLC purity >97%

### Example 11: Preparation of (4-hydroxy-7-phenoxyisoquinoline-3-carbonyl)glycine (Formula 5-acid) Via Formula 7

Methyl 4-hydroxy-7-phenoxyisoquinoline-3-carboxylate (25.0 g, 0.08 moles, formula 7) was added to the reactor at 20-30° C. Methanol (350 ml) was added to the reaction mass followed by Glycine (31.72 gm, 0.31 mole) and Sodium methoxide (13.71 g, 0.58 mole). The reaction mass was heated to 60-65° C. for 8-10 hrs. The reaction mass was monitored by HPLC. After completion of reaction, reaction mass was filtered and dried over suction, the wet cake was dissolved in water (225 ml) & Ethyl acetate (125 ml). The layers were separated and acidify the aqueous layer with conc. HCl up to pH 1-2. The Ethyl acetate (125 ml) was added to the aq. Layer and layers were separated, organic layer was collected and was evaporated under reduced pressure to get (4-hydroxy-7-phenoxyisoquinoline-3-carbonyl) glycine. The yield obtained was 92-98% and HPLC purity >98%.

### Example 12: Preparation of methyl (4-hydroxy-7-phenoxyisoquinoline-3-carbonyl)glycinate (Formula 5-methyl) Via Formula (5-acid)

(4-hydroxy-7-phenoxyisoquinoline-3-carbonyl)glycine (5.5 g, 0.160 moles, obtained in example 11) was added to the reactor at 20-30° C. Methanol (100 ml) and Sulfuric acid (1.0 ml) was added to the reaction mass at 20-30° C. Temperature of Reaction mass was raised 50-65° C. and maintained at 50-65° C. for 7-8 hrs. The reaction mass was monitored by HPLC. After completion of reaction, charged D.M. water (50 ml) and Ethyl acetate (300 ml) to the reaction mass. The reaction mass was monitored by HPLC. The yield obtained was 95-98% and HPLC purity >97%.

### Example 13: Preparation of methyl (4-hydroxy-1-(morpholinomethyl)-7-phenoxyisoquinoline-3-carbonyl)glycinate (Formula 4a) Via Formula (5-methyl)

Methyl (4-hydroxy-7-phenoxyisoquinoline-3-carbonyl)glycinate (5.0 g, 0.0141 moles, formula 5-methyl), paraformaldehyde (1.7 g, 0.056 moles) and glacial acetic acid (25 ml) was added to reactor. To the mixture was added drop wise morpholine (4.94 g, 0.056 moles). Heated the reaction mass to 80-85° C. and stirred it for 3-5 hrs. The reaction mass was monitored by HPLC. After completion of reaction, reaction mass was cooled to 20-30° C. and charged ethyl acetate (50 ml) and water (75 ml) in to it. Stirred the mass and separated the organic layer. Organic layer was washed with water (30 ml). Organic layer was collected and was evaporated under reduced pressure to get formula (4a). The yield obtained was 85-90% and HPLC purity >96%.

### Example 14: Preparation of Methyl(4-hydroxy-1-methyl-7-phenoxyisoquinoline-3-carbonyl)glycinate (Formula 2-methyl) Via Formula (4a)

Charged methyl (4-hydroxy-1-(morpholinomethyl)-7-phenoxyisoquinoline-3-carbonyl) glycinate (1.7 g, 0.0022, formula 4a) and dichloromethane followed by zinc (powder, 1.27 g, 0.19 moles). Charged conc. HCl (8.00 ml) drop wise. Stirred for one hour at 15-30° C. The reaction was monitored by HPLC. After completion of reaction water (10 ml) was added. Organic layer was collected and washed with water. Layer was separated and distilled the solvent under reduce pressure to yield 90-95% Methyl(4-hydroxy-1-methyl-7-phenoxyisoquinoline-3-carbonyl) glycinate and HPLC Purity is >99%.

### Example 15: Preparation of (4-hydroxy-1-methyl-7-phenoxyisoquinoline-3-carbonyl) glycine (Roxadustat) Via (Formula 2-methyl)

Methyl (4-hydroxy-1-methyl-7-phenoxyisoquinoline-3-carbonyl)glycinate (5 g, 0.0136 moles, formula 2-methyl) was added with 7.5 ml methanol and 3.75 ml water. To the mixture was added 1.09 g sodium hydroxide. The mixture was stirred at 20-25° C. till completion. Reaction mixture was added with water (20 ml) and 10 ml ethyl acetate and stirred. The Layers were separated and aqueous layer was collected and added conc. HCl (3.5 ml) in to the aqueous solution at 20-30° C. Reaction mass was stirred the at 20-30° C. for 2-5 hours. Filtered the reaction mass at 20-30° C. followed by the water (5 ml) wash. Wet solid was charged in to the reactor followed by charging of Ethanol (5 ml) at 20-30° C. Heated the mass to 70-75° C. and stirred the mass at 70-75° C. for 1-3 hour. Cooled the mass to 20-30° C. and stirred the mass for 2-3 hour. Filtered the reaction mass followed by the ethanol (2 ml) wash. Wet solid was charged in to the reactor followed by charging of methanol (3 ml). Heated the reaction mass to 60-65° C. and stirred for 2-3 hour. Cooled the reaction mass to 20-30° C. and stirred the mass for 2-3 hour. Filtered the mass at 20-30° C. followed by the methanol (20 ml) wash. The wet compound dried under vacuum at 50° C. The yield obtained is 74% and HPLC Purity is >99%.

### Example 16: Preparation of (4-hydroxy-1-(morpholinomethyl)-7-phenoxyisoquinoline-3-carbonyl)glycine (Formula 4b) Via Formula (5-acid)

4-hydroxy-7-phenoxyisoquinoline-3-carbonyl)glycine (10.0 g, 0.0295 moles, formula 5-acid), paraformaldehyde (3.54 g, 0.118 moles) and glacial acetic acid (50 ml) was added to reactor. To the mixture was added drop wise morpholine (10.39 g, 0.118 moles). Heated the reaction mass to 80-85° C. and stirred it for 3-5 hrs. The reaction mass was monitored by HPLC. After completion of reaction, reaction mass was cooled to 25-30° C. and was added 100 ml water. Solid was precipitated. Filtered the solid and washed the solid with 30 ml water. Dried wet cake at 40-45° C. to get 85-90% yield and HPLC purity >98%.

### Example 17: Preparation of (4-hydroxy-1-methyl-7-phenoxyisoquinoline-3-carbonyl) glycine (Roxadustat) Via Formula (4b)

Charged (4-hydroxy-1-(morpholinomethyl)-7-phenoxyisoquinoline-3-carbonyl) glycine (6 g, 0.0137, formula 4b) and dichloromethane followed by zinc (powder) (2.7 g, 0.042 moles). Charged conc. HCl (11 ml) drop wise. Stirred for one hour at 15-30° C. The reaction was monitored by HPLC. After completion of reaction 2-methyltetrahydrofuran (35 ml) was added. Organic layer was collected and washed with water. Layer was separated and distilled the solvent under reduce pressure to yield 80% 4-hydroxy-1-methyl-7-phenoxyisoquinoline-3-carbonyl) glycine and more than 90% HPLC purity.

### Example 18: Preparation of methyl (4-hydroxy-1-((methoxy(methyl)amino)methyl)-7-phenoxyisoquinoline-3-carbonyl)glycinate (Formula 4c) Via Formula (5-methyl)

Methyl (4-hydroxy-7-phenoxyisoquinoline-3-carbonyl)glycinate (1.0 gm, 0.0028 moles, formula (5-methyl)), paraformaldehyde (0.34 g, 0.011 moles) and glacial acetic acid (5 ml) was added to reactor. To the mixture was added drop wise N,O-Dimethylhydroxylamine (0.69 g, 0.011 moles). Heated the reaction mass to 80-85° C. and stirred it for 3-5 hrs. The reaction mass was monitored by HPLC. After completion of reaction, reaction mass was cooled to 20-30° C. and charged ethyl acetate (5 ml) and water (10 ml) in to it. Stirred the mass and separated the organic layer. Organic layer was washed with water (5 ml). Organic layer was collected and was evaporated under reduced pressure to get crude compound (82-85% yield).

### Example 19: Preparation of Methyl(4-hydroxy-1-methyl-7-phenoxyisoquinoline-3-carbonyl)glycinate (Formula 2-methyl) Via Formula (4c)

By following procedure of example 14, methyl (4-hydroxy-1-((methoxy(methyl)amino)methyl)-7-phenoxyisoquinoline-3-carbonyl)glycinate (formula 4C) can be converted to Methyl(4-hydroxy-1-methyl-7-phenoxyisoquinoline-3-carbonyl)glycinate, (Formula 2-methyl). Formula (2-methyl) can be converted to Roxadustat following example 15.

### Example 20: preparation of (4-hydroxy-1-((methoxy(methyl)amino)methyl)-7-phenoxyisoquinoline-3-carbonyl)glycine (Formula 4d) Via Formula (5-acid)

(4-hydroxy-7-phenoxyisoquinoline-3-carbonyl)glycinate (1.0 g, 0.00295 moles), paraformaldehyde (0.354 g, 0.0118 moles) and glacial acetic acid (5.0 ml) were added to reactor. To the mixture was added N,O-Dimethylhydroxylamine (0.72 g, 0.0118 moles). Heated the reaction mass to 80-85° C. and stirred it for 3-5 hrs. The reaction mass was monitored by HPLC and continued for next step.

### Example 21. Preparation of (4-hydroxy-1-methyl-7-phenoxyisoquinoline-3-carbonyl) glycine (Roxadustat) Via Formula (4d)

To the reactions mixture of example 20 was added MDC (Dichloromethane, 5 ml) and zinc (0.95 g, 0.0147) and heated the reaction mixture to 38° C. After completion of reaction, conc. HCl 10 ml was added and stirred for 1 hour. Methyl tetrahydrofuran was added and layers were separated. The organic layer was concentrated to obtain Roxadustat (compound 1).

### Example 22. Preparation of 4-hydroxy-7-phenoxy-1-(piperidin-1-ylmethyl)isoquinoline-3-carbonyl)glycine (4f) from (4-hydroxy-7-phenoxyisoquinoline-3-carbonyl)glycine (5 acid)

To the mixture of (4-hydroxy-7-phenoxyisoquinoline-3-carbonyl)glycine (Formula 5 acid) (10 g, 29.55 mmole), paraformaldehyde (4.43 g, 147.78 mmole) and acetic acid (40 ml) was slowly added piperidine (12.58 g, 147.78 mmole) and heated to 80° C. After completion of reaction the reaction mixture was cooled to 30-35° C. and 160 ml water was added and stirred for one hour. The solid was filtered and dried under reduce pressure to obtain (4-hydroxy-7-phenoxy-1-(piperidin-1-ylmethyl)isoquinoline-3-carbonyl)glycine (4f) (95% yield).

### Example 23. synthesis of (4-hydroxy-1-methyl-7-phenoxyisoquinoline-3-carbonyl)glycine (Roxadustat) from (4-hydroxy-7-phenoxy-1-(piperidin-1-ylmethyl)isoquinoline-3-carbonyl)glycine (4f)

To the mixture of zinc (0.59 g, 9.18 mmole) and 4f (1 g, 2.29 mmole) in MDC (dichloromethane) was added drop wise concentrated hydrochloric acid at 25 to 30° C. The reaction completed in 30 minutes. After completion of reaction was added tetrahydrofuran 10 ml and 5 ml ethylacetate. Layers were separated and washed with 5 ml dilute hydrochloric acid. The solvent was distilled out. The residue was crystalized in 5 ml methanol and 10 ml concentrated hydrochloric acid to obtain Roxadustat (0.72 g).

### Example 24. Synthesis of methyl (4-hydroxy-7-phenoxy-1-(piperidin-1-ylmethyl)isoquinoline-3-carbonyl)glycinate (4e, Using 4f)

To the slurry of (4-hydroxy-7-phenoxy-1-(piperidin-1-ylmethyl)isoquinoline-3-carbonyl)glycine (4e) (1 g) in methanol was added sulphuric acid 0.01 ml. The reaction mixture was heated the 65° C. After three hour reaction completed. Solvent was distilled out and added 10 ml dichloromethane and 10 ml 5 percent aqueous sodium carbonate solution. The layers were separated. The organic solution was washed with 10 ml water and without isolation taken for next step.

### Example 25. Synthesis of methyl (4-hydroxy-1-methyl-7-phenoxyisoquinoline-3-carbonyl)glycinate (2-methyl Using Formula 4e)

To the dichloromethane solution of example no. 24 was added zinc followed by drop wise concentrated hydrochloric acid (5 ml). After completion of reaction in one hour layer was separated. Organic solution was distilled out under reduce pressure to obtain methyl (4-hydroxy-1-methyl-7-phenoxyisoquinoline-3-carbonyl)glycinate (0.71 g with more than 99% purity).

### Example 26. Synthesis of Compound methyl 2-(((N-(2-((2-methoxy-2-oxoethyl)amino)-2-oxoethyl)-4-methylphenyl)sulfonamido)methyl)-4-phenoxybenzoate (Compound 10-methyl, Combining Compounds 8-methyl and 9)

To the mixture of formula 8-methyl (1.3 g, 4.14 mmol) and formula 9 (1.0 g, 0.0036) in 10 ml dimethylformamide was added sodium iodide (0.05 g, 0.33 mmol) and potassium carbonate (1.00 g, 3.6 mmol). The resulting reaction mixture was heated to 50-55° C. After four hour reaction completion was confirmed by HPLC. 0.5 ml of reaction mixture was extracted in ethyl acetate and solvent was distilled out to yield 0.12 g of compound 10. The reaction mixture was taken as such for next step in example 27.

### Example 27: Synthesis of methyl (4-hydroxy-7-phenoxyisoquinoline-3-carbonyl)glycinate (Compound 5-Methyl, Using Formula 10-methyl)

To the reaction mixture of example no. 26 was added 5 ml 20% sodium methoxide in methanol. The reaction mixture was heated to 60 to 65° C. for 6 hours. The reaction was monitored by HPLC. The reaction mixture was cooled and 50 ml 10% methanolic hydrochloride was added and heated to 60° C. for two hours. The solvent was distilled out. To the reaction mixture was added 20 ml of water and 10 ml ethyl acetate and stir for 15 minutes. Layers were separated. The solvent was distilled out to yield 0.5 g compound 5-Methyl.

### Example 28: Synthesis of Compound (4-hydroxy-7-phenoxyisoquinoline-3-carbonyl)glycine (Formula 5 acid, by combining formula 8-acid +9 to Afford Formula 10 acid and Conversion to Compound 5-acid)

To the mixture of compound 8 (3.75 g, 12.5 mmol) and compound 9 (3.0 g, 10.8) in 30 ml dimethylformamide, was added sodium iodide (0.16 g, 1.08 mmol) and potassium carbonate (1.00 g, 21.6 mmol). The resulting reaction mixture was heated to 60 to 65° C. After 8 hour reaction completion was confirmed by HPLC to obtain compound (10 acid). To the reaction mixture was added with 15 ml methanol and 8 g sodium methoxide (25%) and heated to 65° C. for 8 hours. After completion of reaction the reaction mixture was cooled to 25° C. and 50 ml water was added and stirred for one hour. Thereafter reaction mixture was diluted with 50 ml ethyl acetate and stirred for 30 minutes. Aqueous layer was separated and 35 ml concentrated hydrochloric acid was added to adjust pH 2-3. The solid was precipitated out, filtered and dried under vacuum to yield 2.5 g compound (5 acid) (purity >90%).

### Example 29: Synthesis of Compound (4-hydroxy-7-phenoxyisoquinoline-3-carbonyl)glycine (Formula 5 acid, by combining formula 8-methyl +9 to Afford Formula 10 methyl and Conversion to Compound 5-acid)

To the mixture of compound 8-methyl (methyl tosylglycylglycinate, 227 g, 643 mmol) and compound 9 (methyl 2-(chloromethyl)-4-phenoxybenzoate, 165 g, 560 mmole) in 775 ml dimethylformamide, was added sodium iodide (12.4 g, 82 mmol) and potassium carbonate (155 g, 1120 mmol). The resulting reaction mixture was heated to 55 to 60° C. After 7 hour reaction completion was confirmed by HPLC to obtain compound (10 methyl). To the reaction mixture was added 325 ml sodium methoxide (25%) and heated to 55° C.-60° C. for 8 hours. After completion of reaction the reaction mixture was cooled to 25° C. and 500 ml water was added and stirred till 5-acid formed. Thereafter reaction mixture was diluted with 1Lt ethyl acetate and stirred for 30 minutes. Aqueous layer was separated and added with 250 ml concentrated hydrochloric acid (pH 2-3). The solid was precipitated out. The slurry was filtered and dried under vacuum to yield 145 g compound (5 acid) (purity >90%).

1. A process for preparing Roxadustat or a salt thereof: 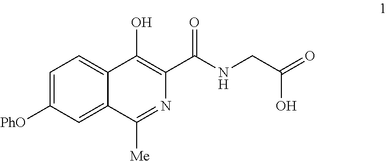 [SMILES: Cc1nc(C(=O)NCC(=O)O)c(O)c2ccc(Oc3ccccc3)cc12]
comprising:
reducing a compound of formula 3′, 3″ or a mixture thereof: 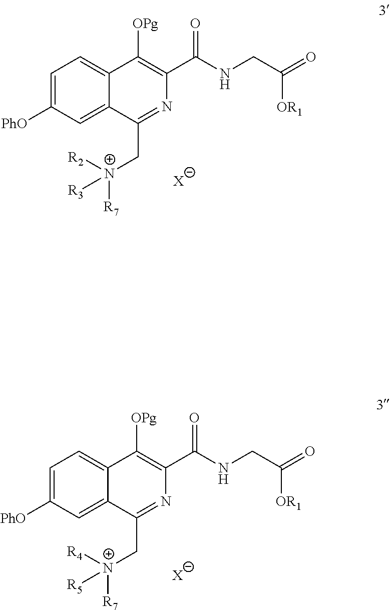
wherein Pg is H or a OH protecting group, R₁ is alkyl, aryl, or arylalkyl; R₂, R₃, R₄, and R₅ each independently represents alkyl, arylalkyl or alkenyl, or R₂ and R₃ and/or R₄ and R₅, taken together with the nitrogen atom to which they are bonded, each independently form a ring selected from: 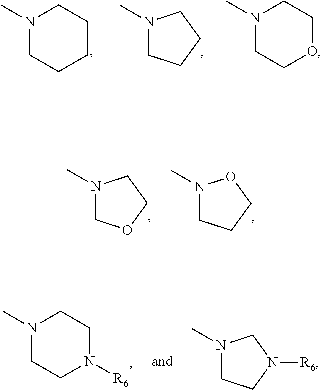
wherein R₆ is H or C₁₋₆ alkyl; R₇ is C₁ to C₆ alkyl and X⁻ is an anion selected from the group consisting of halide, O—SO₄—R₇ wherein R₇ is C₁ to C₆ alkyl, or O—SO₂—R₈ wherein R₈ is phenyl, tolyl, methyl or trifluoromethyl;
to form a compound of formula 2′ 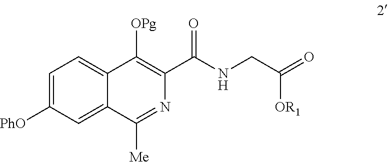
wherein Pg is H or an OH protecting group, R₁ is alkyl, aryl, or arylalkyl; and removing the R₁ group and where present removing the OH protecting group;

2-9. (canceled)

10. A process according to claim 1, wherein the compound of formula 3′, 3″ or mixture thereof. 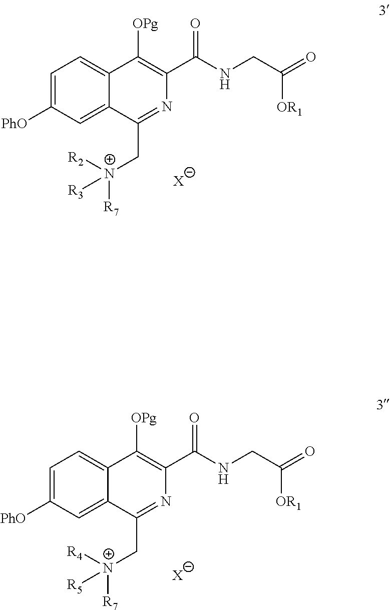
is prepared by a process comprising reacting a compound of formula 4′, a compound of formula 4″ or a mixture thereof: 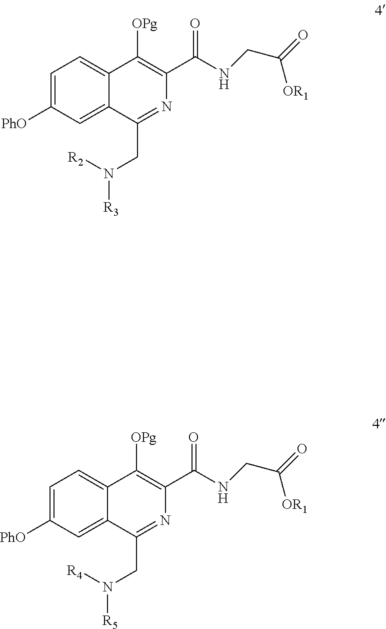
wherein Pg is H or an OH protecting group, R₁ is H, alkyl, aryl, or arylalkyl; R₂, R₃, R₄, and R₅ each independently represents alkyl, arylalkyl or alkenyl; or R₂ and R₃ and/or R₄ and R₅, taken together with the nitrogen atom to which they are bonded, each independently form a ring selected from: 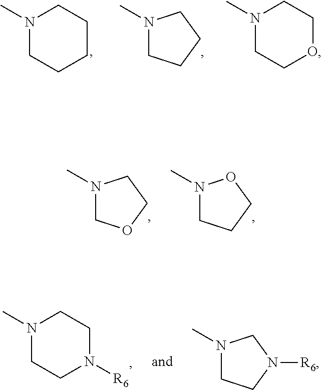
wherein R₆ is H or C₁₋₆ alkyl;
with:
a compound R₇-Hal, wherein R₇ is C₁ to C₆ alkyl and Hal is halo;
a compound of formula R₇O—(SO₂)—OR₇ wherein R₇ is C₁ to C₆ alkyl; or
a compound of formula R₇O—(SO₂)—R₈, wherein R₇ is C₁ to C₆ alkyl and R₈ is phenyl, tolyl, methyl or trifluoromethyl;
wherein when R₁ in the compound 4′ or 4″ is H, the resulting compound 3′, compound 3″ or a mixture thereof, has R₁═R₇.

11-15. (canceled)

16. A process according to claim 10, wherein the compound of formula 4′, 4″ or mixture thereof: 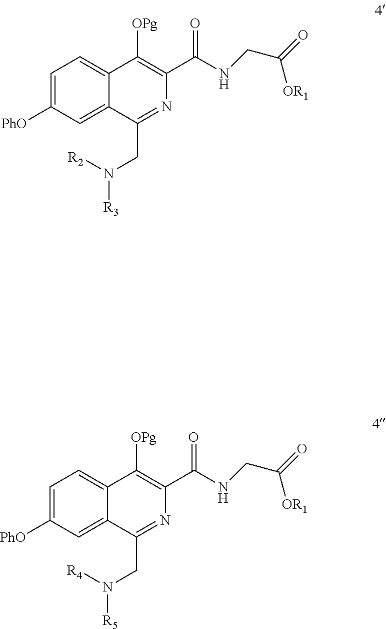
is prepared by:
(a) reacting a compound of formula 5′: 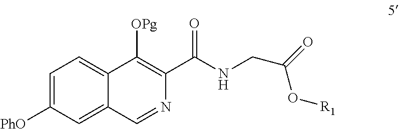
wherein Pg is H or an OH protecting group and R₁ is H, alkyl, aryl, or arylalkyl;
with an amine of formula: 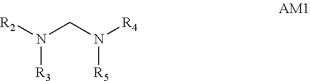
wherein:
R₂, R₃, R₄, and R₅ each independently represents alkyl, arylalkyl or alkenyl, or R₂ and R₃ and/or R₄ and R₅, taken together with the nitrogen atom to which they are bonded, each independently form a ring independently selected from: 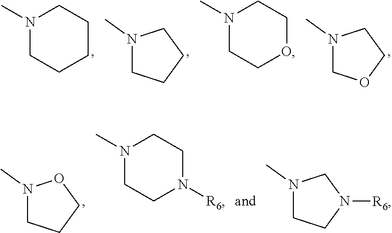
wherein R₆ is H or C₁₋₆ alkyl;
in the presence of an acid; and
optionally, when the compound of formula 4′, formula 4″, or mixture thereof, has R₁ ═H, the compound or mixture is reacted with an alcohol R₁—OH, to form the compound of formula 4′, formula 4″ or mixture thereof, wherein R₁ is alkyl, aryl, or arylalkyl.

17-20. (canceled)

21. A process according to claim 10, wherein the compound of formula 4′: 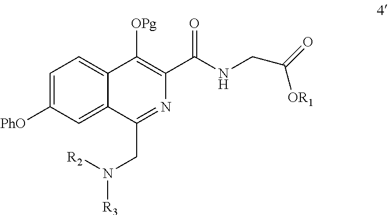
wherein Pg is H or an OH protecting group and R₁ is H, alkyl, aryl, or arylalkyl; R₂ is C₁₋₄ alkyl and R₃ is C₁₋₄ alkoxy; or R₂ and R₃ taken together with the nitrogen atom to which they are bonded, form a ring selected from: 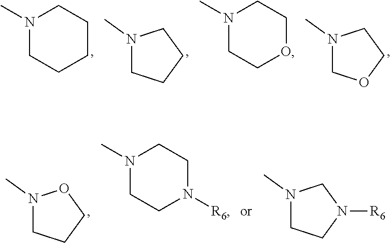
wherein R₆ is H or C₁₋₆ alkyl;
is prepared by a process comprising:
(a) reacting a compound of formula 5′: 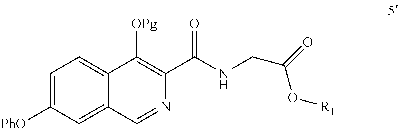
wherein Pg is H or an OH protecting group and R₁ is H, alkyl, aryl, or arylalkyl;
with formaldehyde or paraformaldehyde and an amine of formula: 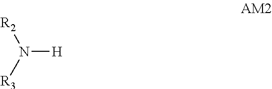
wherein R₂ is C₁₋₄ alkyl and R₃ is C₁₋₄ alkoxy; or R₂ and R₃ taken together with the nitrogen atom to which they are bonded, form a ring selected from: 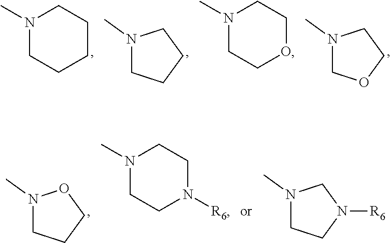
wherein R₆ is H or C₁₋₆ alkyl; in the presence of an acid, and
(b) optionally when R₁ is H, the compound of formula 4′ may be reacted with an alcohol R₁—OH, to form the compound of formula 4′ wherein R₁ is alkyl, aryl, or arylalkyl.

22-25. (canceled)

26. A process according to claim 21, wherein the compound of formula 5′: 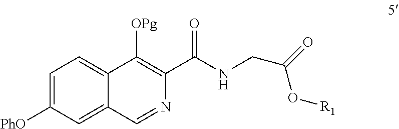
wherein Pg is H, and R₁ is H, alkyl, aryl, or arylalkyl;
is prepared by a process comprising:
(i) reacting a compound of formula 8: 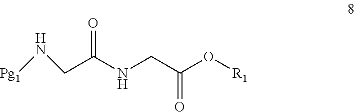
wherein Pg1 is a protecting group, optionally wherein Pg1 is —SO₂—Y, wherein Y is alkyl, aryl or alkylaryl; and R₁ is H, alkyl, aryl, or arylalkyl;
with a compound of formula 9: 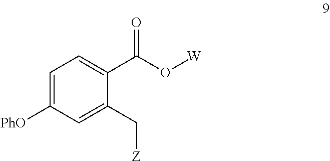
wherein W is alkyl, aryl or arylalkyl, and Z is a leaving group which is preferably halo, mesylate, tosylate, or besylate;
to form a compound of formula 10: 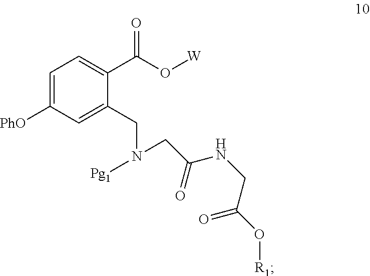
(ii) cyclising the compound of formula 10; and
(iii) optionally when R₁ is other than H, hydrolysing the compound of formula 5′ to form the compound of formula 5′ wherein R₁ is H.

27-35. (canceled)

36. A process according to claim 21, wherein the compound of formula 5′: 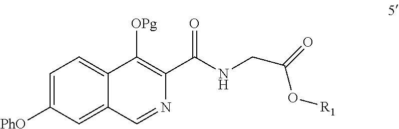
wherein Pg is H or a OH protecting group, and R₁ is H, alkyl, aryl, arylalkyl;
is prepared by a process comprising:
(i) reacting a compound of formula 7′: 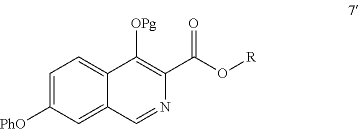
wherein Pg is H or an OH protecting group and R is H, alkyl, aryl, arylalkyl;
with the compound: 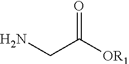
wherein R₁ is H, alkyl, aryl, or arylalkyl;
to form the compound of formula 5′: and
(ii) optionally, when the compound of formula 5′ has R₁═H, the compound may be reacted with an alcohol R₁—OH, to form the compound of formula 5′ wherein R₁ is alkyl, aryl, or arylalkyl.

37-38. (canceled)

39. A process for the preparation of Roxadustat or a salt thereof, comprising:
(a) reacting a compound of formula 8: 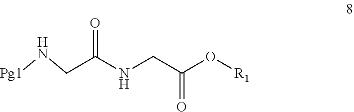
wherein Pg1 is a protecting group, optionally wherein Pg1 is —SO₂—Y, wherein Y is alkyl, aryl or alkylaryl; and R₁ is H, alkyl, aryl, or arylalkyl;
with a compound of formula 9: 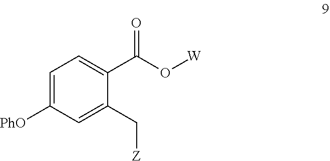
wherein W is alkyl, aryl or arylalkyl, and Z is a leaving group which is preferably halo, mesylate, tosylate, or besylate;
to form a compound of formula 10: 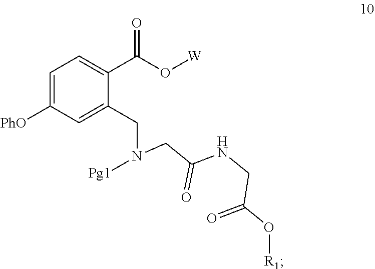
and
(b) cyclising the compound of formula 10 to form the compound of formula 5′ 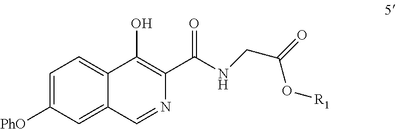
wherein R₁ is H, alkyl, aryl, arylalkyl;
(c) optionally when R₁ is other than H, hydrolysing the compound of formula 5′ to form the compound of formula 5′ wherein R₁ is H.
(d) reacting the compound of formula 5′ with formaldehyde or paraformaldehyde and an amine of formula: 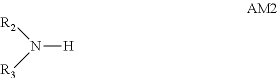
wherein R₂ is C₁₋₄ alkyl and R₃ is C₁₋₄ alkoxy; or R₂ and R₃ taken together with the nitrogen atom to which they are bonded, form a ring selected from: 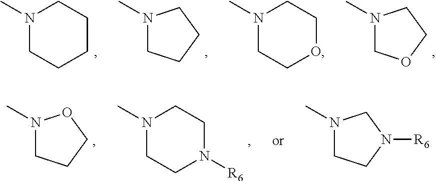
wherein R₆ is H or C₁₋₆ alkyl; in the presence of an acid;
to form a compound of formula 4′: 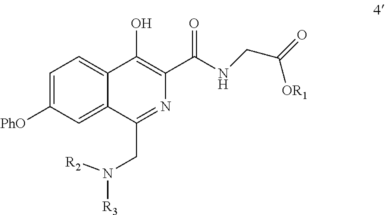
wherein R₁ is H, alkyl, aryl, or arylalkyl; R₂ is C₁₋₄ alkyl and R₃ is C₁₋₄ alkoxy; or R₂ and R₃ taken together with the nitrogen atom to which they are bonded, form a ring selected from: 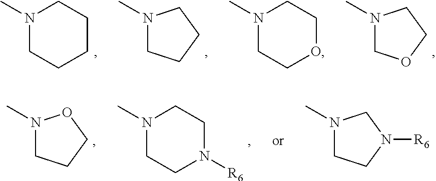
wherein R₆ is H or C₁₋₆ alkyl;
(e) optionally wherein when compound of 4′ has R₁═H, the compound is reacted with an alcohol R₁—OH, to form the compound of formula 4′ wherein R₁ is alkyl, aryl, or arylalkyl;
(f) reducing the compound of formula 4′ to form a compound of formula 2′: 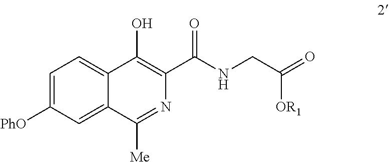
wherein R₁ is H, alkyl, aryl, or arylalkyl; and
(g) optionally wherein when the compound of 2′ has R₁=alkyl, aryl, or arylalkyl, hydrolysing the compound of formula 2′ to form Roxadustat (1): 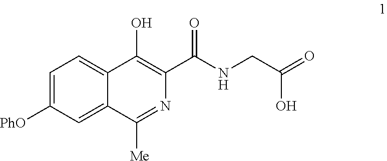 [SMILES: Cc1nc(C(=O)NCC(=O)O)c(O)c2ccc(Oc3ccccc3)cc12]
optionally wherein the hydrolysis is carried out using a base and one or more polar solvents; and
(h) optionally converting Roxadustat to a salt.

40-51. (canceled)

52. A process according to claim 1, further comprising combining the Roxadustat or salt thereof with at least one pharmaceutically acceptable excipient to form a pharmaceutical composition.

53-75. (canceled)

76. A process for preparing a compound of formula 4′: 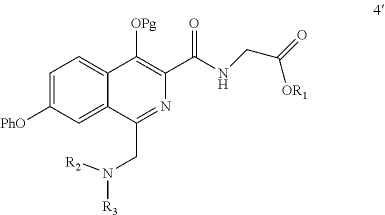
wherein Pg is H or an OH protecting group and R₁ is H, alkyl, aryl, or arylalkyl; R₂ is C₁₋₄ alkyl and R₃ is C₁₋₄ alkoxy; or R₂ and R₃ taken together with the nitrogen atom to which they are bonded, form a ring selected from: 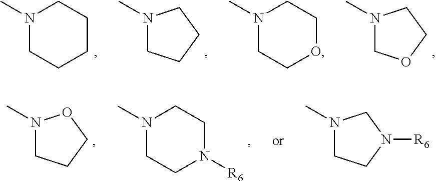
wherein R₆ is H or C₁₋₆ alkyl;
comprising:
(a) reacting a compound of formula 5′: 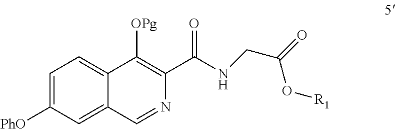
wherein Pg is H or an OH protecting group and R₁ is H, alkyl, aryl, or arylalkyl;
with formaldehyde or paraformaldehyde and an amine of formula: 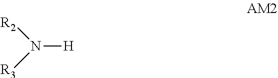
wherein R₂ is C₁₋₄ alkyl and R₃ is C₁₋₄ alkoxy; or R₂ and R₃ taken together with the nitrogen atom to which they are bonded, form a ring selected from: 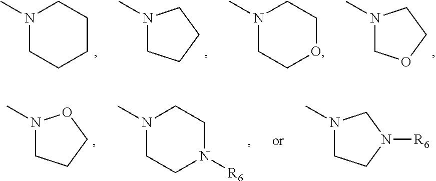
wherein R₆ is H or C₁₋₆ alkyl; in the presence of an acid, optionally acetic acid; and
(b) optionally when the compound of 4′ has R₁═H, the compound may be reacted with an alcohol R₁—OH, to form the compound of formula 4′ wherein R₁ is alkyl, aryl, or arylalkyl.

77-110. (canceled)
